SEQUENCE LISTING (15 sequences)

SEQ ID NO: 1 - DNA, 158306 bp, Homo sapiens
gatgagaattgccaaggcacagcaaagcctgcagcttccatacccgtcatgagagcaaataacttatttgtaaacctaagcacttaattaactaaatggaaagcaaggcagtgagtgattcctgacatggccgaatttaagtcaagctgtgaagctccaggtgcagccagaaatggagatggacaacttgaactgaaccctgagagcttctaaaaaggctttggaccactgcttctcagcagtttttcttgtaagttattcccattatctcaaagaaaaatttaaaatatggttggagaaaagtatagaactgattatccccattttgagcatagatcatcgtttaaccaacttgctttctaactgccaagaaacttgctttggatggtatttccagacataatgtctatcatagcaaatattgacaattgcattttacaaataatcagtttaatatgctttaatatgcttctatttctgattgctcttgatctcatttaatcttcgaagcaactctatcacgttggtgtgcttattaacattattttacagataaagacgcctgtaagtagcttcctcacagggtatttaaggaacttgtagaaagtccacagctagtagctgggagcccatctcatatcaatacactagcttctccccaaagaactttatatatttaggccattgatttctttttttttttttttgagacggagtctcactctgtcacccaggctggagtgcagtggcaacatctcggctcactgccacagctccacctcccgggttcacgccattctcctgcctcagcctcccgagtagctgggactacaggcacccgccaccaggcccggctaattttttgtatttttagtagagacggggttttatcgtgttagccaggatggtcttgatctcctgacattgtgatccgcccacctcggcctcccaaagtgctgggattacaggcgtgagccaccgcgcccggccttaggccattgatttctaaaaagacaaattctcatattggattaaaacataagacccaacaatacgtatgttgcttaaaaataggaatctgttacccaaaaaatgcagaaatgctaaaataaaaaggtttaatgaggaaactcatacgacaaaaagaaaatagttggtaaagttgatagcatataaaagagattttgaagccaaaaacacagggagaggatacaaagaagctcctgattatgataaaaaatagaaaatcaactgtgaagacagaggttactaaagtcagaaatgcaaagagaaatagaataaaaaacaagcaattgtaaagggagagtttaattcatttcaaggctaagaaaaaaatagacaagagtaaaaaataaagacaaatttaaatagacaaaaatttagaagacattgaagacgtgacaaaaatagacaatagaggttatgttatacaattgataagttagaaccaatagacatacactgaactcaataccaacatagattacacatttttttccatatgggggcattggtacatatatactaactctcaaataaggccctaaaaattgaatcaagaggaaaaaatatacatattttctcaccacatgcaataaagctaaatgtgaataagtaaaagcaatgaaaggagccttgggcaaaagaataatctaaccaagagcctttgcagcaaacagttcggggtttagattataaactacattccttactgcagtgtgacctgggcagtttactgaacctctctgaaccttggattcctcatctaaaaatgaggctctaccttgcaaagttgttatgaagattacctataagtatgtcaaataaactaagacttaaaagagagttaaaaccgtaagccgtacaatataactccatttttaatgtactcatatttaactagaaaaagtatctagagtttttgtcgattgaaagcaaaagtctataaaatattcatctaaatactaacagtggttatctctgtgcactgatatttctgccaatttttgttatattgctttatcttttctactatgaatatatagtatgtgaataataaaaaatagtaaatttgaaaataaacatgaacttctgaaaaacagagtacaagaagaaaataaatatttactcttgggtggcctacagatggtggttgcggtaggcatacagccatctcagtttcttgtctgtcattgtaaattctaaaataccctcaatttctgtctttctacatatatacacgtatacgcacacacatatataaaacacacatatattttatatttatatatgttttatatatatgtttttaatatatgttttatatatattaaaaaacacaatattgggataactgacaatttgaatgtggatggtgggattaatcatatggtatcatgaaatttcctgattttactgttaaatttcttgaattttataaaagaaaatatttgtgtccagaaatatatattgaagtatttagatatatatgtatgtatgcagcttactctcaaattgttcagaaaaacattcacatatccataaagacagagtgaatgataactagggaaaaatacaaaccaacagtgcacctaggtaaagggtgtataggagttcatactagtcttttaacatttctgtaagtttaaaattatattaaaatattaataaaaaattacagcagcaacaaattaatgggtatcaatgtctaacaaccacaaaaactttcctaaactacaagttcaagtagaacaggggtctccaacccccagtaccagtgtgtggcctgttaggaacctggccacacagcaggaggtgagcagcagagctttatctgtatttacagtcgctacgcatcagtcacattcacactaaactccgcctcctgtcagatcagcagtggcattagattctcataggaccacaaaccctattgtgaactgtgcctgtgaggaatctagattgcatggtccttatgagaatctaatgcctgatgatctgtcactgtctcccatcacccccaaacaggaccatctagtttcaggcaaacaatctcaggcctcccacttattctacattacggtgagtagattatagttatatcattatttcattatatattaacaatgtaataataatggaaataaagtacacaataaatgcaatatgctcaaatcattctgaaaccatcacgcatccacccccattcatggaaaaactgtctcccacaaaactggtccctggtgccaaaaatgttagggaccagtgaagtagataattaagaacatgataacaacttttgagatgtttataaaattgaaaacaccccatatcaaaagggagggggctttgtataaatcagagcccagaagttaaggcgtaaatctatgtgcttttattttagaaatatgaaataaacatttgacttaaaatcttatgaagacagtcaatataaaggaaaatagataataataaagagtaaggacagaaataaactagaaaagaggaaatcagtggaaataatacataaatttagatgctcattctgtgagaagattataagatagatcaatcccttggaaatacaataaataaaagaaaaggtataaatcaacaaaattaaaaacaaggctagtgatttaacaaccgatacaaaagattttaaaatataaatgattgcaatgcaaaatttatgataatcaacttgaaatgcttagccaaatggacaacacaaaatattaactggcaagagaaaatgtagataacatgaaaataatgatgaaggcaaaggaaactgaaaagctgttatttaaaaaaatctcatctaccacaagaaatattatgacctcaacaaatttactgaacaatattactacatactcaagaaacagatttttctgtattatgtaatcagttccaaatcacagaaaaacaagatctgatacccaattcattctaagagatgagtattatccccatccacaaacctgtgagaaataccacacaggcgcacacacacataaacacacggtcttaggatggcaaatgtgcacaagtgccaattcctataaaactggctcgaaatgattagaaccaggctcaaagatattagcagattttcccaaaacagctgctgaataagtgaatatatgaatacacagatgaagaaaataatgcaggcaattagcaacatctactaaagacctaaaaggcaggaatggcttgtgcatactgtctatttgctaaattttattgatcaataaattttatctatccatgggttactcaaggagcataggcagaaaaatcatttttaactgagcttataaaatacagctataaattaaaacaatagcatgtagccactaattaaggtaaattctactgatgccagaagattataacaaatcacagaatatcttttgtaaaggaattactatttgggctggacccagtggctcatgcctgtaatcccagcactttgggaggccaaggctggtggatcacaaggtcaggagttcaagaccagcctgaccaacatggtgaaaccccgtctctactaaaaaatacaaaaaaaaaaaaaaaaaaaaaaaatggctgggtgtggtggtgcatgcctgtaatcccagctgctcacgaggctgaggcaggagaatcacatgaacccgggaggtggaggttgcagtgagcagagatcacgccattgcactccagcatgggcaacagagcaagactccatctcaaaaaaaaaaaaaaaaaaaaaaggaaaattgaagaaatgaagacattaaggatattcctgaagggaaagactgtgtaaatctcattaaacaaaaatgtaaactctgtgaaacaagacattacaggcgatatgaaagataaaatgcaaactgggaggatatctgcaatgaactaatagaaataaggacaaaagatatgaataagctattctcagcaacagaaatttgaatgaacaatcacacacgtaagaaaatgttcaacctcaccagtaatcagaaaatttaaaattaaaacaatgagataccattttacatccatcaaactggtgaaaattaaataatctgacactcctcaagtgttggtgatgaggtggaaaaactgaaggaacactcacattttgccaacgatactttcacggaagtaatttatttgaagaataaattgtcaacatcttactaactctgatgggacatatatcttacaatccagcaatttcacttctaggaataaactctagaagctttcacttgtggatgatgcaatgtctactgcaatattgtaataggaaaatgaaaaaactaaataacacaagtatccatcaacccaatgaacaaattctgatactttcttaaaatgggaccatttgtaggtgttttatcaacagagataaatctcgaaaatacactgctgagtgacaaaacaagttgcagatagatgtgtagagtataaaagtttgaaaacatacaaaataatggtacaatcatccctctatatccatgggattccacatccatagattcaatcaaccgaggatgcaaaatatttggggggaaaattatacaaaattccaaaaggcaaaacttgaatttgccacatcctgagtaccatgttaaaaccaggcaaatgaagggatgtataggcattgtattaagtattataagtaatctagaggtgatttacagcatatgacagaatgtgcataggttatatgcaaacactacaccattttgtatcagggacttgagcatcagtgggtggtcctggaaccaatcccacacagatactgaggaactactgtatatttttcgtagctacatacatatgctgcaaacatatacacacagagatgagaatgaaagacaccaaactcagggtggcgttgcctctgagacaggaaaaataagatcaggcttgagaagagatacacagttatacaggtacagcactctattttttagcaaaaataggaagcaagtacagcaaaatattaggatttgatggagctgggaagtaggtggtattcaaaactatgcattatacgcatgcgtgtgtttgaaatcattataatttcttaagtgagcacctcagtttttacatataaaaacatataaagtatgtaaaatatataaatatcatgaacagataattcataaagaaagaaaaaaggcaataaaaatggaagtggtggtaaattcaacttcaacccattggttttaaagaaaaatttaaaaatgcacataccattttggcttatcaatttagcataaaattacaaaaatgttaatagctaatgttaacaagggtggaggataggacagaggaggagtacactcactgctaatgagtacttaaaatgacagaaatttacttaacgtagcagtgtatatgtaaaggcaaaatatataggtagatatagatacctagatataaaacctttgccttagcaattctactttaagaaatttatacaaaggaaaacgtggtacatatatgatctgtagtattatttttaaggaatactacgcagccataaaaaagaacaaaatcacatcctttgcagcaacatggctacagctgaaggtcattatcctaagcaaattaacacaaaagcagataaacactgcacgttctcacttataagtgggagctaaacattgagtacaagtggacataaagatggaaataatagacagtggcattctaaaaggggtgagaaggagagggggacaaaggttaaaaaaacctatcgggtacaatattcactacttgggcaatggaaccattagaagctcaaacctcggcatcacacaatatacccatgtgacaaacctgcacatgttccctcagaatctaaaatttttttaagctactaaaaaaataatttatacaaagaaaatcatacatgtggaccaagatttatgcatatgtttataatagcattgttcataatagtgttatcttaaatacagttaatgaaaaaacaatagggaattaagccaattatggtacatctatagagtgtaactgtactctgtagctatttcaaataatgttttaaaagaatatttaatgcccctgaaatatgccattaatgtgttgttaatacacaggtatttaaacactttgtacagtgtgtttacagttattttttttaaataattgtatacatagaaaaaatgacttggggatatccacatcaaaatgtcaacagtgcttatctctaaatgtgaaatcacagacctatgtatgtttttctttttgttcttctttattttcctattttacaataataaacatttattgattttataatagaaaagttattaaaaataaaacgtgcaacaaaataagctttggttgattaaagatttaaatataaaatcaaattatgatagaacttgcagacaactggaaagagagtttatcagctctctggggtaattttaactttctcagctttgaagcaattgaaatgattgcactataaaaattagcattactgaagattttttaagagggaaaatatttctatcaaacattacaaagaattaagatccatcataaataactaattcaaatttgtaaaataaatgtcaagattccaaaagataaatgggtcaaagactggacaattcccataaggggcatacaattagtagataaacatagtggggaaatgttaattcttgccagaaatcaaagacatgtaaatggaagcagtctggagatgccagtttacatattctaatggaacaacaagaaaaaatgtcaaatgctaatatccattgatggtggagctacccagaaatttatccataccttcatttataaccatattgttcatgtccttttaccagaaatgttaccactaagaattgattcttttttttcttttcttttttttttttgagacagagtttcacgttcttgttgcccaagctggagtgcaatggcgtgatctctgcctcaccacaacctccacctcctgggttcaagtgattctcctgcctcagcctcccaagtagctggggttacaggcatgcgataccacacctagctaatttttttgtatttttagtagagacagggtttccccatgttggtcaggctagtgttgaactcccaacctcaggtgatccgcccgcctcggcctcccaaagtgctgggattacaggcatgagccacctcacccagccaagaatcgattcttaagaagtaatttggctaaaggagaaaggtccctcttcatctcctttacttctaaatgttggaacacccagggctcagtcccttgacaccttctcttttccacccagaggcagtgtctgggtgatctcacctatttatgcaggtgagaggagccacaggctctctcagcatgggctccagggagagaagaggccagagcagaggttttcctgggagtggggtaggggtagcttgagggccattcaaggactctgacctcagtcagagagggcaatgaaggaaagagagaccagcaaagacatccagagagaagcccaaggtgaacttggtcatcatattaacccctatgcaataacaaaacatccaggcttgatctaccagagcttatcagtgaggtccccaggaattgcctaattgcagtagccctcagctcacttgcaggccaccagatctcagctggccctgaccaccatctccaaagcctgaaggaattattgtgagacagcccactgaaggtgggtacctccaagagccctgccctgccctgccagaagccagagctgtccatctgttggtggtcaccctgggaatctgggacaagaaggtgcactctggtctgatgccagaaagcagaacctcttgggctcccaaatctcaggatctttttgctccaccaatgtactcatccactcatcccagcctggggcctctgctttacatgggtcttttcattgcttaaggatgtttgggcatctaccatctcttcttcttcttcttttttattttttttttgagacagagtctctctctgttgccggggctggagtgcagtggcccaatctcagctcactgcaacctccacctccctggttcaagagattctcctgcctcagcctcccaagtagctgggattacaggcccacaccaccacacccagcttatttttgtttttttagtagagatagggtttcaccatgttggccaggctggtcttgaactcctgacctcaagtaatccgccctcatcggcctcccagagtgctgggattacaggcatgagccactgcgcccggcctcatctcttctgcttatgaccttgtgaggcaggcatcccaggtttcattatccccgttgtgtaaagattacgacttacctaaagcactgctggactcaaacccagatcttctgccctcgattccatgtccatctctgtgaccccaggtttggccaagtgtggatcttgctgcatatgaacagcctgccatttccttccttgtgtacacactgcctgtggaaaatggacaagggaagttacatttctatcaagagctgaaacaagcaattttgctttagagatgatatgtaacagaaagaaatagtcctggaagcagggagccctgtatgcatctgcctgtgttctgctcctggctgacccctgagatcgaatcatgctgtttgctaatgtgtggaaagtatgttttgctctctaggagtcctccccagagcctgacagattcggtgataggaatagaactgtggagtgaagatggctctctccaggtgtcactccagttgccatacatgtaaagaaagcctagacactgtgatagctttgtcctcctatccctgaactttgcccagaggcgaagcttcttccccaatccttgctcaaatgccttctttttcctcgaagaatccaggctgttgagcaaagagggagatcctgggagcttcacccaaatcatctggaagtttcttggggtgccagatttgaaataggagctgatagggtcaatccatcaggcaccctgaccctagacatacaggaattcccctctgaggttgaagaattccccctccatcagacccaaaagtgaaaaacaaactttgcaaaggcacgtttataccaactcttctggacggggctgagtagagttttctctggtaccgggcacatgctgctcacagactctctggcccgtggacatggcccctggggtctgagaatgcagatgggggagaggctcaccgaggagctctggatgacctggctccagtgtgtcacctcctggccttggcctcctaaccccagctgggctgagaagaccccaaagagggtctttcccactctcccaacctgttttctctggtgattaatatataacctgagaaaatgaatcacttgtttaaattcagagttagctgggctaagacaggtgacaagaattactcaaaaacaagaaagcaatattgggggtaaagatgctttctatttttcttttctttagttatttttttgttttttgtttgtttgtttaagatagagacaggatcttgttctattgcccaagctggagtgcagtggcatgatcacagcttaccgcagtcttgaccttctgggctcaagccatcctcccacctcagcctcccaagtaactgggactacagctgtgcactgccacactggctaattttttaaaaaattttttatagaaacagggtctcactatgctgcccaggcaggtctcaaactcctgggctcagccatgagccaccacacccagcttgctttcttttttaaatagaaaggaacacataacaacttgattttaaaatcaagaacaggaagcatagactctatactttctttaaaacattgtaaataagtctcctattctttactacttcagttaaatgggcaggaaacaagaaagggaaggatcaggtgagggatgggagaaataaaaaccatgccgaaaaactgcagtaatacataaaaacactaatggctgatacgtatttattgggcactatggtatgcacagttcatggtttttctcattttatctccccccaagccctgtgaggcaaataattttactactcctattttaaaatgaggaaactgaggtgccagctattaaataatctgcctgcttaatgggactaaaaaccgagaaaccaagcttcgaaatccggttctcagcgatcagagtcttcactcaggaggcaaaaatgcagcaagagacagctttttcctccagaaagaaccttatcctgttatcatcagaattctgtagctaaatacttcctttaccattttgtaacaatactaaatatagggaacattttgtatttccatggaaaaggatgtattgtctacatacggcacagcctccctgggttatttgattctagctttcattgcccttgaagcattttgcaactctgtaaattgcagataactaatagcaatgcaaaatgcaaattctgtcctatactgtagaagttacactgacttcactgccacactgcagaaaaagctacttctgtatccatttgccatttgtaccttcattgcaactttttttttctttttttttttttgagacggagtctcgctctgtcgcccaggctggggtgcagtggcgcgatctaggctcactgcaagctccgcctcccggcttcccgccattccctgcttcagcctcccaagtagctgggactacaggcgcccaccaccacgcccggctaattttttgtatttttagtagagatggggtttcgccgtgttagccaggatggtctcgatctcctgacctcgtgatccgcccgcctcggcctcccagagtgctggtattacaggcatgagccaccacacctggccaacatttctttactcatataaacttgtgtggttttttggcttctcctttgaactggttataacacccttaccacttgcctcattaattagttaagtaaaatatttaccatgtactcgtttttatatttgagagtcatgattttaccttcgttttttaaaaaattatctcttaatggaattatgaaagcatgtcaaaagtatcttgcttgttactttctgaaggaccccaggcatgatctaactcaaatgaatgacttcaggcgtaattggataactgatgatgttctcagttgggtcctgagaagcacagccttgtcctgacaacttcctttcaaacactctgctctatccgggacaaagcatcatggccaaaggccccggtcccctcttcctggctggtcttctttactgttgattgaggaaaagtaattggcatataaattatatgattatgtcccattataatgtttccattatttctaacttctgctatgtctcgtagtttatctcagagctatcgccattaaatagctgagaaaaggcttgagaacatttgttttaaaaataataataaacatgtgatgtgatggttaatattgaatgtcaaattgattggatcgaaggatgcaaaatacttttcctgggtgtgtctgtgagggtattgccaaaggaaatattagtaaaatttgagtcaatggactgggagaggcagacccaccctcaatctgggtgggcaccatctaatcagctgccagtttggctagaataaagcaggaagaagatgggaaggacttgacttgctgagtcttccagacttcaccattctcccatgctggatgcttcctgccctcaaacatcaaactccaagttcttcagcttttggactcttggacttacaccagtcgcttgccaggagctcttggcctttgtccacagattgaaggctgcaatgtcggcttccctacttttgaggtttgggaactcgagctgacccaccactggcttccttgctcctcaacttgcagacgaccaatcatgggactttaccttgtgatcgtgtgagtcaatactccttaataaattccctttcatatataaatgaaatgcacgatatatgtctttatacgcagagcaagagcatgacacaattccatctttttaagcaactgatacacaaagatctcacagccaatccctactatacttggcaaaactactttttgttgtatggactctgaagtcaaacatcattcaagtctcattccaccacttactacagtatgtatttgggcaattcagtaacctccctgagcctcagtttcccctgggggaaatcatgctacatcataggattcttcctaaagtgtgaatgagatcacatggagattctggcactgtgcaggcactttggacgctggctcctgaattgttaactcctgacttttaggcctgcctaggaaaaatgatgaaccatgtcagcaagataccctgtcttcagaaaaatgatgactcattggcagctcttcagaacttgaaacacatcattgggaatgaagagaataaaggggaattaattccaggtttccggcagccatggatcctgtgtgctgaactcagcaagcctttgtgagatgacgacgtggcatcctgctgcagggaggcttgggccgttgcaatctgaccagtgcccagccgtccctgatacccagggtgaggactgtgcccctgaacaccccatggttggcagcactaggaccaagagacacagccagtgtgctggtgcccccctccccaaactgtccttgcttgttgcaagcagctagttcagttcatggcttgcggggagtctccctcaggagccagcatggtcaccagtcacaaagcctggccagcaccccagcagctggaggaagcagacacagagtagacacttcacgaattctagtctacactccccctcttccccacaaaggggaaaaccctgggcaaactttacggagccaacaatactcactatctacctggtgttcaaagccaacttgaatctttatttccttttgcttcaaagcataatacaaagagtgaaaataggttttccatcctactggaatttctaagatcataaaatgcattgctggttccacagtatctagtcataaaaaagactgctctggttctagggtgttgttgttgtgatgatgattttcccaattgttctacaatctggcagctgaaatcctgataaggagagtccgtgggccaacacagaccaggaaacataaaaataagaagaaaggggatctcaactctttgctccctggctgcataacttgagactctgggaatcagaatattagatcttccagggacctagcgtccttatatccaatctatatcaattagatatgtgaccttgcgtaaggaatatccgaacctcagttttctcatctgtaaaatggggaaaataattcctaccttatttgttaagaaaatcaaaagaggcttgactatgcagagagttgttataagcattttcatgtgataataatttaaccttcataaccccatttgacgggggaataaacaggcagagataggttacagaatttgcccaaggtcacacaacaagttggagacagagctgatgcaaagacacccctggtcaccaggagtgataatcttggatgaataaatacctatttgaaatgcttgaaacacctaattcttccaaatagtctaaaatgtcctctgcaatcttgttttcacccttcctttgtttatcaggtctttttttttttctaacacagataaggaaaaggttaaactccagtccaaccactttccaaatgaggacgttccaggctaactcaaattaacgaggtttacctacttgttttgccatattccctcatggcgcaagaatgtccacacagatcagtgagtgacccaataaaaacttggtcattctcctcatggcaccctggccccaagcaggagtataagaggatagagtcattttcctataacatgatccaaggaaattcatttcatcttggaagtggactaggctgatgtgcagagaaacctgattcagtcccagggaaagccagatacatagggattaaggatttgtggttgttaagagaacagaacttggcctcagatcccctgacttctcctctctctgtcttttactggtgagatacagtttggtaccagttcccaacctcttaaatctcaactgcaaaattaggaaataacagtgtcaaattttattgtttaaagtattattttattataacaccaacaaaagcttgcctttctcaggtaagatatcccagttcctgaagatgaaaagaagaaaacctgattctgggtataaaattctgtttctgtcatcagtgaacttcaacttggtcccctggcctcttcctcttgggatttgcttgctttatccttgtaaaatatctgtttcttctctcctaattaactttcttatcaccatgacactaaagaaaaaggtcattctcaggtcatcaaatgccactctcttgaaatctttatttattcaagaagctctgtaaattaagacagagcaagaactgggcaatgtttaaaaacctaattataaaaccattattactttagatagaggactgcagaccagagtgcaatgtcacccaaaatagaaacgtctttaaagaattgcaaaggaagaggccaggcatggtggctcatgcctgtgatcccagcattttgggaggctgaggcaggcagatcacttgagctcaggagttctagaccagcctggccaacatggtgaaaccccatctctactaaaaatacaaaaaaaaaagcaaaaaaaagccaagtgtggtggcacaccctataatcccagctactagggaggctgaggcatgagaatcacttgaacccaggaggcagatgttgcagtgagccgggatcatgccattgcactccagcctgggcaacacagcaagactccatccccccaaaaaataaataaataaataaaagaattgcaaaggatgaagccacagagctctgctaacatgcaccatgctgatcactccattggaggctccaaactctaagaggctcaggcttgcctcctctttagctaataggaaatgaagaggcctcaagaaactgctgggcttccttgggcatggaaatgaccaaagatgctatgaatggatctattgactgctcagatgcatggctggagtcaagcacatgacagtcaggtctggcagccctgggagccattctgtctctctaaggcagaggctttacccattttttgacaacacatcttatccccaatatctatttatttgtttataaataatatagcactagtatattccgtagtaccacctgcaaattatatagtacttatatgctataaacattatacgtgcacaaacatgaactgtctaaggaaatacaaagaaaaatctgaaaggtctatattattttcttcttttgccccagaagatctctcaagcaccattctgaggcaatggttctcaaactttgtgtaaccaagaattacttggagatcttgcttaaaatgaaggttcactgggccttgaccctagtgttgattccattactcagtattcctgggatgggcccataagttgtcattgtctgagctccccagctgactcttattctcatgctcaattggattttgcagccactgatccagacccttgtgttttcctgccccttacattttttgctacaattatatcactgtggtcttattgttatctacctcctggacaccttcgtgatcccctcagtgaatttaaacattaaaaaaaattagctaccattaattatttcctgtggcctaggattgctgagggcttcacatgcattgcctcacttaatcagactagggaggtattattagtcccattatacaggtgaagaaaactaaagctcagtcacaatgtcttatccaagttcactcagctagtaagcacaagtgacagactaggaattcaagcctgtatctgtcggattcccaaggctggactctttttttttttttcttttagagatagggtctcactctgtcacccaggctggagtgcagtggtgtgatcatagctcactgcagcctccacctcctgggttcaagtaatatccctgcctcagcctcccaagtacctaggactacaggtgtgtgccaccataccaggctaattttttggtttttatgtttatatagatggggtcttgctatgttacccaagctagtcgcaaactcctggcctcaagtgatcctcctgcctcagcctcccaaagcactgggattacaagtgtgagccactgcccactgcacccagcctcaaagcatggacccttgaccactaagtatattgcccacaaaataggtcattataatttattgaccctctactatgaaccaggcacttggcttgacaccttacacacaaatctctaatattcatggcatacctcttttcctagaggtattaatttcagtccagagattctgatgtgcaataaatttctctattctctgtaacccatctaaattcctgactcttaacccctattttcaggccttctggatttcttctccccaccccacccccaaccccagtagttatgccatagttctggacaaatacaatagccaggcatggggtcaggggagtgtgacctagtcctctgtcatcagtccacattggtggccactgaggggcccatcctcccatcaacatgacctgctggccccagagactcactgctctcccaccctacatcaagccaggtatatcttttgcccatgtgccaacacagccatttcctccctgggttcttctcttccagcaggatttctgggaagcaaggtactgtcccctctgctctcagacccatagacaaacctcccccttccacaccacctgcaaagcccaagagaatgtctttctagtgatggcaaggtgaggggttccttctcccttcacatcccctcaagaaatgttagtcttttgcttttctttgttggctagaattcacctgcccacactttgggaggccaaggcaggaggatcacttgagcccaggagtttatggccagcctgggcaacataggaagaccctgtctctaaaataaataaaataaaataatataaaataaaataaaattcacttgccctaaggtcctatatttttggaagacaaaagatttgtagccttctctctactttgcccctcttaagtggaagggagacagtttgggagaaaaataagttgatatctcaaaatattatctcagatacatgttgtccccattttataggtgaggagattgaactcattaaggttatataatattatatacagcaagtagataggcctaggacccccccagttcaggaaatctcatactctgtgaagtcctcccctcttccctgccagtagcctccctggtcagcttcaagaaaacacaacagagcttgtgcattaaaaccaccactggaaggtcagacccggtgatggggcaggctggaagagcttaggtagaggtgaaagcagacaacaccctgaaaggggacaggtacccactcctccttgtcattttccaggagagccttatagaatttgagttggcatgagatgatgttagaggaatttggggtggtgcaagtaaaggaactacaagagctagaaacaatcttgaaggcttcttcccatgttgaaagtgttcttaccacgtagaaagtggtaagaaagtgtactctgcagaaatttggggggcgggtgggggcacacggttggtatttcagaggcacttggattcactggtgtcttttgatgagtttgtgtcccgaataaaatggggagtggcggtggttactggcagtacccagaggagaggagatgagggtgggcacaggggagaggagcccgagatgggtagccagctggtcttacgtgccagctttataggaaaaaaagtggaactttcagattctgcttaactgtgcaggagccagccaagtatccaaggcagaagtccttgctcagaaattgtatatctgatgggcacggtgactcacgcctgtaatcccagcactttgggaggccgaggtgggtggatcacaaggtcaggagttagagaccagcctgaccaacatggtaaaaccccgtctctactaaaaatacaaaaattagccaggcatggtggtgggcacctgtagtcccacctactctgaaggctgaagcactagaatcgcttgaacctgggaggtggaagttgcagtgaaccgagatcgcgccactgcactccagcctgggcgacagagcaagattccatctcaaaaaaaaaaaagaaaaagaaaaagaaattgtatatctgacgggatggtccgcaagcttcggggcttgggaggaaggtacagtcataggacagaacacagctttagactgggtcaagatcgtccaagtctggtggtggcttcagaaggccctttgaatgaggctcgtccaggcaataattaccatgttatctaattttgcccgcttaacgtttccacttggatgtctaagagccatctcaaagccccacgtggccaaggctgaatctctaatcttcccccaacagacctgcttcactcacagcttccccttctcagctgacagcaaccccattaatctgcttgttcaagccaaaaaacctggaagtcatccttaactcctctctttctcccacatctcacaactcagcagtcagaaaatcctgtgggctctaccttcaaaatatatccaccacccatccaccactccgcagttgccctttaccatgctgggccacgccaccatcacctctcactgggccactgtaatagccttctaacagatctccccactgcaccctggctcacttgcaaaccagagtgatattttaaaactataatacaaatcatatcacccttctgctcagagtcgccccccatgtgtcaatgcataaaagtcaaagtccttacaatgtctccccattcattggcctccttgctattcctggagcacaccaggcatactcctacctcagggcctttgcacagactgcccgaagctctcctccccaagacgtccacaggggcaactccttcacttccttcaaacctttgctgaaagtttaacttcccaatgcaattcactctaaccactctgcttaaaatccctaccttctccccaaccccctgcttttcttgtagtggttcccattacattctaccagtctacctaatttttttttttttttttttgagacggagtctctctgtgtcgcccaggctggagtacaatggcgcgatctcagttcactgcaacctccgcctcccaggttcaagcgattctcctgcctcagcttcctgagtagctgggattacaggcactcacctccacgcccagctaacttttgtatttttagtacagatggggtttcaccatgttggtcaggctggtctcgaactcctgacctcgtgatccgccctcctcgcctcccaaagtgctgtgattacaggagtgagccaccgctcctggccagaaatctcttctttattatgtctactgtccgttatccaactccagaaggtaagaacctccactgatacataaggacttgtataccccacgtgcctgcaacagtgcttggcacctagtaggcataccaaaatatataaatgttgaacaaatgaagaaagttaaagtaaaactagaggtccaaaaatatcacaaaagccatctatggtcgccttttccctacctgattttgctgagtggccttacttttcagtcctctacacagctggaacattaatgaacacagagggggaagaagtgtgtttactctaggatcacctctcaatgggtcacttggcaagggcatctttgcttcttcgtcagctccttttgacacgggggtgaagggttttctgcaccacactttgaccacaagcatcaccaatttcactgaacccaacagaaatttggaccctctgggggctctctgcgtggcagggcccttttctttttctttgggcttaggctgcaatttgaaacaccactttcctgagccagcatcccccttgcagcgctgtcacagggaggcttaggcagccacgtggaagccacctaccccgacctttggcagaatttccaaacacaacacagtagctttaagttgattaatttggaactctgaccttggccccaaaaggtaagaatacataacaaggtattttattctcaaaatgtgtcaggataagaagcacttctgtaaatcgacctttttaaaatagatataattagatttgcagttgggggcagtaaagaaagggtctgaacagtggataacatgttgagaggttaattattaatgggcagtcttccctaacaaagtatctaataggcattgtggtctctttggcttcagaaattaccaagaaagcctggaccccgggtgaaacggagaaatggacacaagtcccctgtgtttctccattctgttggttttatgcatctttatccaatcaagtgcccttggacaaagcctgaaaccaggtaagagcctgacttttctccagagatgggcatgaacttttctttttaaaacgtgtgtcacaaagaatccaggggtttctgactgtatgggacagagcttgcttcagagcgtgccaggtggttctatacacgacctcacaggttcacagggacacgtagccgtttgtaaacagaaatgaaattttagaaaatcatcctcggattaaaagtcttctaagagtgcctgcagctagcagtgaagtctcttgcgtatctgatatggaaacagtcatgctaaaagttattcagcggcaaatgatatgaaagcgattcagatcttcctgagagttaatgtggcatctaatttgcaaccggggagccaggcttgcaatgaaatgctctgtcgggttggggttgtctaattgcctgcctctctcagaagagaggggaatttatgagagttctatttccagtcaggtctaaagtagagatatcattgcacaatttctagactttgttttgctgccaaaattttagtctgaggctgaagaatccaacagccgtcctgcctgttttgtttgtaaattcacaaactagattgatggcgaaagacagatcaaatgccttatcaggaaggtagtagagtggagcccagcttccaggctaagcagtggaccaataaatgacggatcagttaattgaacaatttggccagactagacaggtgtttgggaatttgttttggtactggttactggtcaggagctagtaacgctaaggtagagtctgagatttttcttttctctaagactagccttggagagagctccgacaactttgagaattagaaattccctaacgggatgtacctaaaaatttcagaagcttttttcccctttaagaaaaaggctgttcctttgtatttgactattttacttcataggccagttaataactgagtagcagtattaattattgccattcactaagtattttctgtgccaagcactatactaattatttttgacacatcatctcatttataaagccacatccaaaaatgacccttacataataatttagagccatacaggtaaaattgctggtaatctttattcccttggttcatagtgattttgctaaagggcctttcctgccctttgccccggaatgttgtatttgtttgtttactttttaatgaagtttataacacacttctagaaaagggcctctatcataaatcctcaatgagttctcacaaactgaaccagtaccccaaagccttccttgtgtgccccttgagccttaacatatatgattttttcctttggggagatttttcattaatagaaacacatcagttgctcttttgcgtctggcttctttcgttcgacattatgtttgtgaaagtcaaccatgcagtttcgtggagttgaggctcattcattctcactgctgtagcattccactgcgggaccatgccacacttcctttatccattctactgtggattggccaggactgtttttgcaatggagccatgggaattggaagctgtcagtgctgagatagaggttgaagggagggatggcctcccccaggcttctgggtattgtccagattttccttgagcaatctcattgtcctgatcagcctgcttctttccccggggcctagtttgtgcaaatgctgagagaagtggccactattagcaagagggttcctcagaaaagaggatccaagtcaggaggcctcttgttctcttggaagcagaatctcttggcagaattctttctgcaatgagtgagcccaatctgtgtgcatgtggctggtatgatgtctttctggggaaaccgcagggaagccggtgtcctgtggtggagcccttgtcaggaaagtttctacaccaatggttctcaacaggggcaattttgccctccaggggacacttggcaatgtctggagacatttttggtttttggttgttaagaccagggagagagtgcttcccttccagcatctagtgggtagaggccagggaagctgcttcacatcctacaatgcacaggacagccctcacaacaatgaattgtctggcccaaaacattattagtgcagaggctgagaaaccatgatctatgccaagcttcaactgtcactcatcgagcaccaggaagtactccctcaaggacccttcctctttagcccctcctcccagcctacttcttttgagtatttattattaatgacatcttactaatggagtcactaatgtgctgcacacccagcatgtgccaggcactccacggacattttatctcatcctcaccatatggttgagtaagctggcagttaagtgccttgccggcggtcgcatagctggtcagggcagagcagggagaggtacctgcagcctcaatctgtctactgaggccgcagtgctctgtgtcgagcccacacccagtgatttgacagccagaggtttctagaaaaacattctgctcagttcctctcacagagagggagatttagacagttgttcctgcccttcagcactaagggggactggatagcgtgagctgcttgaagcccacatggcacatcacacttatgcccagccccaatgcctgagacccaggttccccagtcggtgttgccatgagagccaacgctgctcctagaccctggcaagtcccaccttcccggtactgggcccactatcaacaggcaccttctccctcccctccaccccagcctcagcttgcaaatcctaggagcagctgaaagctacctattcagtcagtccctcatgagtgtcacaagcatacatgagcacctgccatacgtaacgcagtgcctgagtacaactcacacactctgttcacacgtttcctctcttaaaaatcactgaggaccccaaacagtgtttattatgtgagatatcaatatttccttcattagaaattaaaactgagaaatctttaaaatctgctaacactttttaaaatgataataaacccttgcatattaacacatataatgtaattttatgaaaaataacttgtattttccaaataagttttaaatggagaagagtgacattgttttacaaagaaatgttgcaaatctctctaatatctggcttcccaagacagctggattatcttacctgtttctgtactcagtctgttgtgatgtattgctttgtttgaaatgtaggaagaaaatctggcctcccatagaggaaaagggaggacatcctaaggcgatctccctggggccttggcctgcaaaccactgcgcttgtgcagtgggcactgtacaacagggggctgcacagccgtccccaccttgcaggaatttacagtcaaatggggtgggagtggggtggggagggaatagggggccatgggactgggtggaggggggatgtgccatcaataaaagagcaaccgtctgtggagaatttaaatacaaatataaccaaaagctgatctgcctgtcattatcacaattataaagaatgtcagccgggcgccgtggctcacgcctgtaatcccagcactttgggaggccgaggtgggtggatcacctgaggttaggagttcaaaaccagcctggccaacatggtaaaatgccatctctactaaaaatacaaaacattagccaggcttggtcatgggcacctgtaatcccagctacttggtacgctgaggcaggagaattgcttgaacccgggaggcagaggttgcaatgagccgagatggcgccgtcgcactccatcctgggcaacaaagtgaaactccatcccaaaagaaagagtatcagcgataaaggcaaagctctttccagaaaaaaatggttttatggcctaagttctaaacaatctccgtggttactgttggattttatgtagtaagtatatgtaagcttcaaattcatcacatctttattatttatcctttcataaatattgtgttctacattgacattaatctggagaactcccggttatgcacttggccccaacatgcacagactcaggggcgtgttcttgttgcaagagtaagtgtgcgatggttcagatcctttgacgtccttatgtctccaacctcagtggcacttattacctgcttctgcatgtaaacagtagatttgaaacaaaaaatgatagcgtagtggttgtacaaacaaaaaaccagaacttgaattatttcaattctctcatcctaggtgacccctgtaagcttctcatatgtgtttaaaatttaaagcaacaaaacagattgtgaacaatgagttgtaacatcttgtttgataggtaaaaatttggactcatacgaaaaatatttttactgcatttgaatatcttaaagttaaaatgtatttgtctttttaaaaattgttcactgctttataactaaagaagaaatcgagaaagtagtgatgattgctgatttttacataaatatgactgaaaatacttttgtcttatagagcagagggtcattaaaatgatccgttctgtgtgtcaaatacatgaggtataccactgagtcacatatgtaaatgttatcagctagaacagaacatggcatgagatgaggaccatgagagaggaatgagtagagaagcagattttacttccaagtggagggaagaaggcttcttggaggaggcggcatttgagtagggtgttgaaggatggtatccggacagaagagaatgaggctttccttgtacaatagcagaggtacacagctgataaagtacatgtgcttttcaagcccaaatacctttcaaaaaagaccatctaggaagaaaaagaaaaaatatcccatagaacggtgttgatagcaacacttgttaaactgcaacaaactggaaacaatgtgaatgcccaagtgcaaaccactgaagttggggactccagctactgctaagtccaacttcgaacaggcatggttttgaatacctactactagaagtcacagaaacttttgggggcaataatgatgaagttcagaaacaaaagcctattgctggcccctaagcactcacagtttaacaccagggatcttagcacagaggatgggtttccaagagtcgttgagcctcctgagatggtgcccaaggctgtgtgtgtgtgcattttgagtggggagcaggtgtcataactttcagcaaattatcaaagggacctgtgacctctgaaaaagctcagtaacaactggtcttgcagctttgtaaaacaagtataagaaaattgtatatgcaataatgtagggaaaaaaaaacaggactaaataatctacatgttgaatcataattatgaaaacactgtagacacatgctgtatattgctagagattctaagtatatttaaagatatataaagggttttaaggtttggaggatttaagtacttaaatgtacaagaaaaataaaataaaaaggccctgatcagctacagttagattggtcatcccaagacgacccccgggaaagggattttccactgtccttccgacctgccaccagcacccaactttgcaggttcagccacactcaaaatcactaccccagcagggtctgtcagatttctgagaggcctccacagttacatggaatgtcacccatgactaatatttgagatagagtccggagatgaaactcattcttccaagagtgcgtactattctggaatcagcagagggccagcccccagcattccagggaaattctatctttcccatgcaagagaccctcccatccatggccatccttcctagccctacagctgagcaggtctgtggtcctgctgaaaggtccaccctggagagacgggggacagcagcctgtcctccactctgcttgcctctcacagagacccaggactggaacccatattctttcattctgagctggagagagaaactgcagggctgacggttgaaggcatggggtctgtaagctgacccgaaaacatgaatcgccaaatgagtgcgcagccaaaagccaagagaatctggtacctggctacccacgatggccctctccctgccctcctctccacatcagctgccacagtgctgcgggcatgctgggtcatcacagttggcacagcagggagagaggcagaaagaggctgggccccgagacagggcagaacagtccccatttcatcatgaggggaatgcaaccacccaggcctcagcctgggtgtccctgacagccacgggctccacgtagaacagaggtcacagcgattctcacagccaggaaggtactgtgcgctggcccccaggacagtgaccagtttcgagtggggcgctctcagctgggaaatggggtccaacccaccagggaaaagggctgactaggggccaataggaccctccagatccctgcccagcaggctggacttcagtcccaaggtgataactagaacaagggggccgagcctagtctgtccagcagcgggtggaggagccgcggtaagaaaggcagtcgggtcttacattcaggtacctacttggggcgagaagaccaaatccagtcactccagccctggaggagtcagggcgcccacccggctgccccacagaaggtgcaattcctaaacatggtcacaaagtagcgcagtgacatctcagctgcttcggagttccaggagagggtttctggctcaggagaatggagtttctgggtgactggaagatgaggctgttggctaacttccctttccttcccacttaggggtctatttccagaagcattcctcatttcattttttccccagtggggcttggctaggggtggggaggcatggggaaaaagtgaagagaagagaaggaaatgcatggaatgcaagcggaggcctctttgtctacagcgttttcacctccacttgtgcaggttcatattcagggcctggtgaagtcagttctggcacggctctccagtggactggcagtggcccttaaaaatagttctcaggaagatgtgtaggtggcgacgctgagtagtgtttatgtcccaacatgaagagcaggcctcattgtgtgtgtcctgtgattacaactgtgtgaaaatacatgtgtttgagaaaaaatgctggaagaagatgcatgaaaatgtttatacgtccgttgagtttgtatctgaacttttgtctcttcccctctttatgtaatgttatagtatgtatatggtatattgactttcatgatttaaataatcagaaaataaatttactacgaaatgataaaataaaggagtttgactagatcagtgatcctggaactgtagtgggtaatgaagcacctaaaaggctgctttttttgtttgtttgtttttcctgagacggagtcttgctctgtcgcccaggctggagtgcagtggcacagtcttggctcgcagcaacctctgcctcccgggttcaagcagttctcctgcctcagcctcctgagtagccgcgattacagacacacaacaccacgcccagctattttttgtacttttagtagagatggggtttcaccatgttggccaggctggtctcgaactcttgacttcgtgatctgcccgcctcagcctcccaaagtgctgggattacaggcgtgagccaccgcccccggccagggcttattaaaataagattctggggccctaactacagagttgctgattcagtgagcctggggtagacttggatatttacgtttctcagcagttcccaaatgatgctgctggtgtggggaccacagagaaccactagagaaaaaacaaaagaaccaagacttgaaactaggttactgacttccaggctagggtgcttcctacagaaacaacacgaggctacagggcaaacctcacttcacttctaactttctagggttgaaggggtgagaatcaacatccactgctcctggatactgacttcttccccagtagctacaaccccaaaagtatttaaaaccccaagatatttgtgaaaatgtatttgtccaaaataaaatatctacatgctaaaaagaaagcagtccttgaaatgaacaaaaattcccaagctgagcattacttgtaattggtagaaaaataatcattgcaaaagctaattaaccatccaccctgagcggctccttggcaggcctgaaaatatgatcaggaaaattcttttcctatgagtgaggcattatcaaagcagggcagagcctatgggttatccgttgactaggactgacctttccgtgactgccaaaaactttccagtgacaaatgttccagcttccttgtcccaagagaaactaaagtggtaagaaaacaaactgctttatactgaaacataacaagatcaacagcagctgtgacagcatttattaaactgtccgctgtgtaccaggccccatggtcagtaggaaagtatatccctgagggtacaaaaatgaaaaagcgtacatccactcaccaaggacttcccactgtgccagggtctctagccccttatggggtgtgtgtgggggcagggaggttggagattgggagaggcgcataatttcaacacaatgcaattagtttgtaaataacactacatgcaaaatgttacaagagccctgaggcagcgcaggttagctgagaagcctttacagaggaggtgatatctgagttgaatcttgaagtttccttcaagaagtggaaggacattgtaagcagagggaaaaaaaacatgaaccagccttgcttgactcagaggctggaaaagggagagtgaagttgggaattcttgagattttggggtggccctggatggggtacataaaaagaagtggccagagggaaggacaggaagggccatcctcagaagttttgtcctcatcctggaggcagggcagaccactgatgaggggggttgtttttaaaaggctgcctttcccagatggattgatagacttatctcagcctggctaaaaccctggccactccctgatagagaacaaggttcccagcaggcaagggcaggggtggggcccagcagggaccagttttgcaagctcacctggccctacacccgcaaacactcagcttttcctccaagacaagcctcaaggcttggagcaagtctctttgaagttttcttatgaattgctatgcttaacactaaatcatgggaaagattaacacccatgaaactgaagcaggagcacataacaggagcttaatatgaatcttccatcttaaaaatatcttattctacatctgaaaagagaaccatttggaaattccacgtcatactcacttatagacttaacagcaatttcaactttaaaaattcattcccacctgcattatatgacttgattcttacagtagccctgtaaagaatattgggtaggaattcttattcccattttagagacgtaaacattcaccatccgcggaccatggggaacacagctgacatcaattgcaagtcagtttctgtggtttactgcagcaagggagagctcactccagacttgggcttgtgctggttgagtctaaggaaggggtggaaagcaaggggaattctggattggattctgtcaaaaaggagtccaattcaatgattcggcatcataataatttaatagttatcactaattattatttaatataatcaataaatgatctagcagatgggaagatgaaaacagggctgagctggctttgatacagcaacacaagtcattcctgttagtcagaagagggaaatgtttagtcatgttttttgattttggagtggcctcctccctgtcttgatgcaaacacagatgtggagggtctgtgctgcacatgtttctgttcggtgggcattgtctctgttcacttgtgaatattatggtctggctgacggcagagctggtttttgttgctgtgctgtgttttcgtgtagtctctcccaaagggacttggggaatgcgtttgaggtctcttaatacctatgccctagctgcacagctttgggtgatgccctttacctccttacctctgatctctcaggagtaatagtgtcccttccacactgacattctgggtgtgaatgggactgattagaatggtcatcgtggtgccaggagatctgcagtggtgaccttccctggtatcagctatttaaatattatcaggaaagagaaatgtggaagcaaagttctatccattttaagcatgtgaccttttaccctaactgaaccttaattgactcatttgcctctagcaattcaaagattcccagacacccggaggacaaagatgggattctgtaagaacagcccacgcagtcaatgacccttatgccatttcttgcccggaactgttttattcagggcctgacatgcagcagtgtttgattaagtgctgacaatgaaaataggtatttgatgctgtttggaagaaaactcttacatttcttgaattgaactaagatgtacagaacaacagaaaccattatttttaagaatatttaaatgtgccaagttctttgcctatttatgtcaatgaatccagaaaaacaccctgtgagataggtattatccccacttcacagatggggaaattgtggctcagagtgttgcaaactctccttagaccactcagctaataatttcctctctggtatagctgaaacatgggcttgcctgaagggcagaggcagaagaagcctccaaacctcccaaggtcctgtgattctcattgatgcctacttctagtggttctggtgtccctctcagagcccacatgggtatctgagccttcagagatatctcatttggtttggggggttgtttttggtttttctgagtctcgctctgttgcccaggctggagtgcagtggcgtgatctcagctcactgcaatccccgcttgcagggttcaagcgattctcatgcctcagcttcccaagtagctgggattacaggcatgcaccaccatgtccagctaatttttctgtttttagtttagtcggggtttctccatgttggccaggctggtcttgaactcctgcctcaagtgatctgcccacctcagcctcccaaagtgctgggattacaggcctgaaccactgtgcctggccgagatatcccattttggataggggagcctgggacatattctcaacttccgtgggtctggccactacttatcagaagtttctgctcaccttacacaactgtgagtgatggccaggagcagcctcagtggcccaaggggaaaaacctctggatgaagccctggacccctcacatctcagcctccatcactgcccctggctacccaaagtcaagcccagaaacacagatgtgagcacaaacccaggcagcccatagaaggcacagccctgtgatgtcaagcttaaaaattctgggctacttttaaggtcaccagggatatttcgaaagagctgaagctttttcctccatccaagaatcctctatcttccaggttgtttttatgagctgaagaggaccctccacggctcctcggtgacttagtagtatcaagacaacacctgagaaagctcctttcatgcaaatctctgtctcacgcatgtgaagagaccaccaagcaggctttgtgtgagcaacaaggctgtttatttcacctgggtgcaggcgggctgagtctgaaaaaggagtcagcaaagggtggtggattatcgttagttcttacaggttttgggataggcggtggagtttggagcaatgttttgcgggcagggggtggatctcacaaagtacattctcaagggtggggagagaattgcaaagaaccttcttcagggtgggggagattacaaagtacattgatcagttagggtggggcagaaacaaatcacaatggtggagtgtcatcagttaaggctattttcacttcttttgtggatcttcagttgcttcaggccatctgggtgtatacgtgccagtcacaggggctacgatggctcagcttgggctcagaggcctgacagtctccttcctgcctgcgacacaggtctaggaattattccacagttgggcactcttggtgtattattgttcatgccctggcccccacctttgtgccgcatagagggactccccttcctgaggttaatgattgggaaggtgttaaagcaggaagaactgcaagaaggcactcgtgagtgagcctatccactcattcctggaagataaacaaacgaccagtgtggctctccatactcagagggtggtttctggatgtcagagatttcgccacacaccagctctgtgccaggccttgtgctaggaccccaacatggtcactcgatattcacacagtctagcagtctgccaggactgccaggacagaggaccacagactgggtggcctcaacaacagaaatgtattttctcacagttctggaagtgagaagtctgaggtcaaggggtcagcagggtgggtttcttctgaggcctctctccttgtcttctgatggctgcgtctcttctgtctgcacacagtcttctatgtttctgtgtccgatctcctctttttataaagaccccagtcatattggattaaggcccaccccagtgaccttatttcacccaaatcacctctttaaagactctggctccaaaagcagtcacattctgaggtactgagggttagaacttcaatgtatgaatttgagggaacacaaagttcagcccattacacccagcaaacactttgctaggccctgctgtgacccgggtcctgggcttggcactgggcatacagtggaaactgcaaatctctctttgttctatttgagcttcagcaactctccggaaggcttccgcgttagaaaaaaggcttacaaatacacaggaaatttgaaagtgagtagtaagtccacaccaggcccagacagggggcctgtgctgaaaggtgcaggaagcagcagccctgctgagacacacaggagccggctggccggccgctagcagggtgggatggggagtgggggctcagggagcctgggctggggagaggcatcctccctcttcaacagaatgattgggactttctggcagcataagcaataaccggcaggagaccaagaaggcgccgtttagctggctcatgtgtgagtgagtgatgaaactcaccctctggcttggcgccttatggaactatcaactctgcatgggtgtgctcagtgtggaccccaaacctgctccctctggctttgagaggtatttctcccatacaggctggcttcagccagggggttgcctagactcgcccaggcctcagacgaggggaaagggccaaggccggctgccactttctcatcatgtgcctgcctcccttcccacatagaagggtcaagcccaaattagaagggtgggatgatggctggtgcctgaggacagagctggggctaggaggacaaactcctgggctcctcttcatgtctgggctacaagatcctggcataaacaggggctcctggcacagcggaaataaaccatgggtccctgagggagactctgggtgagaaaagagacccagatttggagttgaaagacctgggcctgagtcccagctccatcacttcccagcacgtggccttgaacaaatcactcaaggcctgcagacctcggtcagtcattagtaaagagaagattactattatagagaccccactgggctgttttgagggtgaaatgagaccacgtaagtgaacgcactttgaaactatccattgctggtcacaaaactaacgtgtgtacgcatctatttactaaatagtcatataatactcacccgattcatatgctactcccagtgtttttctctactttttattacaaaagtgttcaactacagaaagaagtttaaaggatacaatgaatttccatattccctccagatagactcaataactgtttgcattttcgtatatttgctgtatgtgcatatttatatgtgtgtgacttgtaaaaccagttgagataagagggggaaaggaagagagaaaaagacacacacatacatatcccatatgaaagttgtggacatcatgatacttcaattctaaatatttatacgctagtatttctttctttttttgagacagagtctcactctgtcacccacgctggagtgcagtggtatgatcttggctcactgaaacctccacctcctgggttcaagcgattctcatgcctcagcctcctgagtagctggaatgaatgcctcttactaaggacattctcctacatagccccagtaccatcaccacacctcagaaaattaaaaataattttatactatccaatatccagaccttactccaattttcccagttgtcccaagatagttgttgatagcgtttttcagactgggatccactcaagatcctcctgttgccctgtttgctgagcttctgccatgtctccccagtgtctgaggttgtgagcatcttggttacttgctgccaattctcacactcatctgtgctgcccaccgtcccatggcctcttgcggacctctctcccatggcgctggctccttgcacccagctatctgcttgcacacctctctccctgctggaggataagcaccccaagggcagtggccagtcttagctactgttgtttccccaggacccacacaggcctggcatggagcaggcacgtggcaaatgactgttgcatgtttgaatcttcatcagagggctaagcaacacacacctttaaaaagtgagtgagttttggccaggcgaggtggcttatgcctgtaaccacagcactttgggaggccagaggcaggcagatcacttgaggttaggagttggagaccagcctggccaatatggtgaaaccccttctctactaaagatacaaaagttagccaggtgtggtggtacgtgcctgtagtcccagctacttgggaggctgaggcaggagaatcactcaaacctaggagggtggaggttgcagtgagccgagattgcgccactgcacccagcctggacaacagagctagactccatctcaaaaaaaaaaaaaaaaaaaaaaaaggaaatgagttttaagtaatttcatctgacaaattctgtacccctgccggagctcatggaaagcatcccagagacccaggtgacctagctcaggccctgccctacttccccatctacaagtggccactggggctctgaaatcgcttggactctcgactagtctgtgctcgacatctgtgacaggaccatcgactgagagggagagctgccctccagaccacaagtcttctctggaaaacacacacgtatatgagaagttggctgtgttcccattataggtcttacagaagtgattccaagtcttgtgagtttctgtggaaccagaaaaccacttccggaccagcaggagtgagaagaggcgcatgcatatcatgtttgcattgttttcttgcatgtgtcacttcaccacaatgctcaggacctcaatgagagttttaagttctcccagcttcaaattgtgctttccaggccagagcttctcaaaatttaatgtgcctttgaatcatctggagagcttgatacaatgaatgtttttttaaaagtagatttgggatgggctccaggatcctgaattcctaacagttggtctgggtgacgccagtgccactggtccctgaactacactttgagaagcgaagttctaaaggtcacaaaacaacttctcatttcttcagtgaactggtaattagcacctacagctaaatataaggcagtgttctagcactggaggaacaaggaaatgggaagaaatacaaagaagaataagacacaggcctttgactcaaatccaactggatatccctttatactacgtctagcattttgcttaagtgggacctaaaaagttactccaagctgcctcccctagggtcttctggctgtttctggtaatgtgtattcctgctgatgctcaagccctttgcaacaagttcctaacatggtatcaaatactgggcacactagcaaacttcagccataaattagggcccaaatgttgtcaccaaatggctgtgtgactttggataaatcactctcctctcacgtggactagggttgggagctcttcaagtcccttcctgtctgccaaaaggtttctaagcagtttccaagccgtttcttgcagttttcccatcaagccaggtgaaggcaagtgttatgtccccatcagggtcccccagccccctcttcttagctcaggactggggctttctggggctcacctacagtcttcctcctttccttcctaacttcacacgcatttatcaagcacatactctgcatcaaacctgtgctcaaggacagggaggcaaaggtcaataagatgcactccctccctggggaggagacctaaaacagaccgcaggtgccctgctgcgtggctggctcaggctctcagagtgtgaaggagagcacctcactcagcactggcagaggccggcaggcctaagtgggccttcaccatatgccaggcctggctaggagcttccctggaagattccaggtaacgcccagcaaccctcaaaggaaaatgctagtattgtccccattttaaaaatgggaaaacaggcttaaaaagtcagatgtctggcccaacatcacacagctaatatggattcagtgaaatatatgcaaaaatactcagcagagtggcaagtagtaactaaacggtaactaaaatacctttttcaaatgacagaacagagagtgggggccagtaatctgcactctcagacaaaactgacaagtgggagaaaggcttgtctaccctgaaggacaaaaaccaggcttgggactggccacacatgacgagaaaactgtgagaaaagaagcaggagagactttggaagagggcttggcaggccggaaagcgctaggtgcacacgacgggcagtgggggctcaaacacaggtgggagcccaggggaggagggagcgattagtcatgctggggaggctccaagggccctttgtccttcctagatggctactcagacgccagtctgcgagcaccccacgctcagctgctccatgcttcagccaccatgcaacccatctggataatggactgttatgaagcatgttggcagccatcgattaggactaaactctatgctcattgggttgtatttgcaaggatgctctgctaagatccacagcagaggtgcatctctctccttctcatttgatggcttggaattaaaatgccacccaacattctctgccaaagacatgaagatttatgggtcactgtcaatatcaacacatgctggccattatttgagtctccttgctgcttgggactgattgctatttactttgaaagataaatgtggctttgtaatccatgcaggttgctgggaggaatgggttttaaatgcatttgttcttttttttttttttaagacagagtctcactctgtcaaccacgctgtgcagtagcacgatcctggcttactgcaaactccgcctccagcttctcctgcctcagcctcccaagtagatgggattacaagcttgcgccaccgcaccctgctaatttctatatttttaatagagacagggtttcgccatgttggccaggctggtcttgaactcctgacttcaagagatcccccctcctcggactcccaaagtgttgggattacagacaggtgtgagccaccatgcccggccttgggttttaaatgcatttgtttcttcccttggagacctttctattccactccattctgctttgtctggtgatgttggtcccaccctgtacccaccatcagatattctaatgtcctccgaggctctggaacaaagaccagagggaccagcctgtcctctgtccagagtcatcagtatgacagtatggagagacccttagcaccttagtagaaaaattgcccctgtcttatggggtgacaccactcatatatgatctggggcaagagtcaagacccccaaggagcttacttaaggagcaataggttgtcccctttgcctcttctggtctttcgcgtttctgagcgtggtgtggaaactgagtcaggagctaaatcagttaggtcactggttctcaaacttgagtgtgcatcacaacccactgaagagctttttaaaacacagattgtggcccccactctagagctcctgattcaataggcctagggtgcggcccaggaatgtgcatttctaacgatgtcccaggtgatgctgattctgttgctggtcctgcatcccacctgaagaactgcctcagaacaagagtgggaagcattagagaccaatcatgggagagatgaggctgctggcagtgtttggcacttgggaaaaagaacagcaccgtctaaaggagaattttattaagtttcagtgaacaatgtgcaatttaaatctttttgtaggaaatcgtcagttaaaggtcaatttccataagtatgtggatctatgtattttggaaatgcattgaaatgttacatgggtctgggaccacacaagctacccactggtggtctgctatacctaggtactaggaaggtcaaggtactgagtaggtcacagtcttgccaccgggatgctttgctaactttattgagtgaaagtagtaagaaatgcctggtattgataagagcccaccgtgctagtttttaccaatggtccctaagcgattttccctgcagcagtcagggtttacaggtaaaactgggcccagggcaataaaggggtacagtctaaggactctgggagtttcctgatgaatactagaggtctttggaggcagacaactttttctttgcagcgggacttcacattataagccatgatgaggctgcaattgcgatttatcagtgctgccttccttccccaggcaacgtactaggaagaatcatggtttactgaaggcaggctaccctccccatcaaagcctagtggactgggcaagcaatcggttgggattcattacttggaggcatgtatccccagagcaatcaaaggccctgaccggcacaggattgggaggctgttgctgagacccaggcctcgagtgacaggtagagcagtggggtggaggaaagtcaacatatttggagatacgaaaaaggcaggtaagtaattgtattcgttgagactttgggtcacaagaataataatccaaaccaaattaacttgtggggaatttatttactcatgtaacatagatgtccaaattataagcttcagggacagctggatctatgaccttaataaagtcaccatgtttcttcattcgctccccacccccttctcccttcataacacttcagagtgccagattcctcagtccttggaactcaaacttccagagagaccacacttcatcacatacctatggcacatgttccagaaatgattctggccaggtgtccacccctgaatagatggccacaggcaggagaatgggatgtctgattggtctgatttgggaaacatcctatccaccacttggataggatcaactttacccaaacaggactgatcaggatccacatgggaggttcgctcctttggaggaggagatgccagcagaccgtggaggactggctgtgaggcacgaggatgagcgggaggcaagggcggctcctgcttccacagaggcgagcaggaggatgctcctgccctgctcacacagctgtggctctgcaccttgctccccgctagaaggtgggctatcgtagctaagctacctgggcagagcagacctcgtgagtgtggggaaaatcctgggcagcataacagactcatttgtagggcaaagggattttcagttagttctgtaacacactggagaaccaacacaatgctcaagcatcgttcctcccagaggaagcagatgggtgagataaataaaaccacactttcattggcatcctaggatgctcggggccctattttcaaggctgtgtatgttgaatacacatttcagtgatgctttagtctattccacagtgattctcaaattgtggcccccaggccaacagtatcaccatgccccgggaacttgtttgaaagacaaattctccatcccactcaagacctcctgaatcagaaactctgggtatggggcccggcagtctgggttttaacgcaccctctgggtgattctgaaacaggcaccaaagtttgagaacgtctgcctataaatgtatttgatccaatccatcaagttcaaagggaccacctactataaaaactaaggcttatgttcagggtttattgagcagtctgaatttagaagagtgtgaacgtgaaacagttttctatttctcccattccagccttcagccacgcactatcaagttatatgcttccatccctttatagcatcctgggcagattcctttaacttttggtcagttcgattattaagttattttactttattctaaagcctcaatctcacatctgatttgacattcttcccttctctcatgagctagactcaaagaagatggaatctgcagtggggaggggataggatagttctgctctttcactcctcttttctctggagcaccgtggggtagggtggagtgggatggggtgggagggcagggtgaggcctggtcctgggattgctgcccctcatttttcagagcccaacccccatgggactgggagctggtagtgagggtacagctgggggacacagaggtcctcatctcttgtgagtggtctgctcttgctctggttcactgtggtcccatctgctgctggtggccttggcctgtgaagatgtggatgtggttttctgagggccctatcggtgtgagcccagcccatttgtcactaaggacagctgtggccccttttggcttctccaaaggtccatgcccctcctactctgtcctcctgtacaaagtctcagaaatgacttcggtcttcttccccgcctctgaggattgatgggagctgaggtgtggggacccatcccctcaggtacactgcctcatagtatttcttctccccgcttggctgagcctcctctcttcagatggctaaaacaggcacaacccccacgttgccacacagtcccataaactcctatggatacaggtcagtctctctccaaaactccatcctcatatccccagcaccacatgggacctagaatagcaacgtctcagccagcatacagcctggaacaatatgaccatttctgcctcctgcaggcaccctcattccgcattctcggtaagtgattctaatcggtcccccaacttgaactctagagactaacaatgggaagaagagaactaaattattctcccagcaattcctactctccctctcccacttgtctgtctaacatgggctgggggaaggtggtgatgggaatagagactgcttctttgagagccctggagtttgaagaatttggccccagttgctggcaactctcattacagagtgtgggttccgtacattggcgcggtggcttatgcctgtaatcccagcactttgggaggccgaggcaggtggattgcttgaccccatgaggtcaagaccagcctgggcaatatggtgaaatcccaactactaaaagtaaaaaaaaatcagccaggcctggtatagtcctagctactagggaggctgaggtgggagaattgcttgagccacagagagcgaggctgcagtgagccatgatcatgcacagcaagaccctgtctcaaaaaaaaaaaagaaaagaaaagaaaaagaaaaaaaatggatagaatgtgggttccttagcacctgttgtttcagcagatgtcctggtgccacatttgctatttgcaatgcgtcttcacatacattatctcagttgaccccacagtgattctgtgaggtgtttaacctataaggaaacaggctcagagagattaagtgacaagcccaaggtcacacatgcagaggtgatggcaagatgggaacccagcccagtgactcctgagcacctgttctctccctgctgtttcatgcaggtgcctcagccacacttgtcttccagctctgggtgaacttggagcaaataaccagagaaatagcttgtctccaaggccacagagcagctggcaggggagagtttggggcaagggatttccttagggaaaacaccaattcgttctggtcatgcccagtcacatgggtcccaaccatcaccttcccaggccaaggtcttaggttcccctggaacccctttggctttactcccttcctgggctgggggaaagggtggggatataagctggtacttccaaagcagaaagctggggtttggggctgacagagctgaccttgagcttgagctccatggagtacccagcccaggtcaaatggggtcaggaagccatctgtgccttctggatggctctgtctgaatgattcacttctgcctatggtgggtggaagttcatggttttactcccacatggtgaccacagtgcatggcaattttctagtcaacaggtggggttggggcctctgctacacacaagaggcttctttcttcaagtttaagacttctgatgctcacaagaccctcccatgcccttaccacgtgcagcgctcacccccagggcctggggtttggtgagatgtgtcgcccacatgggctgggatcatattttccccagggaagatgaaagccctcaccctctcacaaagccaattgctaaattttcaagaattttgcaagctgatttttaaacatagccattattttaaaattacattatagaacttacaatgaaacaaatgtttaaaatgacagtaataaatactcaaaactcctctgttctcattttactattgtctctgtctgtaaggttatttccgtctgtcgtactgtgtggtggaaatactattcaatgatacaccaaagtgaatctcttcccaactccgttatcagtaacatcacagtgggagcttaacagcagccatgatggctgtatttatacatggacattggcaaacattatagattagggtttgatgtattgttttgttgactgtgtaaagcaaggttcagcaaacttttctctgtaaagggccagacagtaaatatttttccttttgtaggccataggtgtctgttaaaactactcaactctgctattgcagcacaaaagcagccacagacaatagacacacaaatggacctggctgtgttccaataaacctaattcacaaaaacacatggtgcaccagatttgtcaggccatggattgctagcccctcatctagacttaagagagccatagaaaaaaaatgttagtaatgaagattaaatttagaactgtcccaattgtaactagcacaaaacattaaggaaatattcttccagtgctcaaaaacaatgatttgattgagcaaagaattcatgtcattgttgaatgattgagtttcaatgtgtccttatttcatttttgtcttcttcattaatgtaaataaacctgtcagccaacctacacttgtcatttgccaccataggttgactagtgagacaagggtccaacaaacatcagcaaaagtattctgtgaaaatcaattgctatttggagtttacaataaagaatactgtatatttcattattatttataaattgtataataccttgatagcaatgaaatgcacaatatatgcatgtatgcgtgtatatatatatgaatatattttcttccagagagccagttgctgagtatccaccaatgcaccactgcctgaggggtgtctacataaaaagagacctaacagcccagagcagcacttgttaaggacccaaggcatggtccagaccccaaggaagggaagggggtcagggatggatagacaatctggaaagttctccaaggtggtctgagaaggcttcatgggggacagagagccgtaaagcatggtaggactcaatgacaaatgaagaatggctgtacttcagacaggcacacagcatgagcgcaggaggcgaggctgaagtcagggacaccgagacaagagggtgagcatagggaagcagaagggaatgaagtggggtcaaatatatgtaatggcatggaaacatgctatgagatcaagccaaaagaaaagaaaaagtagaatgcaaaattttacatttgcattaaggaaaaaacacgttagaagaaaatgcacttaaataaattgctctgtgtgatagtatgatagctgattatgtatttatcctcaatgctttctgtaccttcctgttgttctgaagttgtattactttgatattcatggggaaaaataagcttttccagtatgaagggcaaaaagcactgagccaaacctgaagccatagctaaggacagggcacatctcccctgatgcagcccagaggcccctggagggaggtgctggggggtgaatagagactaggggtgtcactccctcctaggaggactgcaggagcacatgggtagccaatgctaaggcctcctgcccccatgggcctaagtagccctgtccacactggggatctgcccgctacctcatagcctcaggtagatgtctggtctctctttgcaaatgtcccccgcaacccatcacattcataaaggcaacaaattggggaaaacgagaggtcctctgtctttttagggcttcaccactgctgttttcccaactaaagaaatatgtacatatgtttaagtatactttaccatgcgtagaatgggaaaaacagaagttattctagagaacatctcgctcaactcgtttattttactaataagaaaatagaaactatggaggtcaagtggcatacccagggaaacagagctaagtgagaactaggctccagcagagatgatgcccagggccgggcacctcctttgtcagcatgcaggtatcatcagacaaggccaaaacagcactgggcgatgtgggctgagcagcccttggcttcgaaaacatgatttttttattattttttgagacggagtttcactcttgttgcccagaatggagtgcaaaggtgcgatcttggctcaccacaacctccacctcccgggttcaagcatttctcctgtctcagcctcctgagtagctgggattacaggcaggcaccaccacgcctggctaatttttgtatttttagcagagacagggtttctccactttggtcatgctggtctcaaactcccgacctcaggtgatctgcccgcttcagcctcccaaagtgctgggattacaggcgtgagccaccgcgcccggcagacatgacgtattctaagcactcctcaaatttacctgtggacagcaagaaggcctacaaggcaccccgtttacgatatgccacttggctccttcagggcagagctgcagcagcagtccttgtttctctgtgccccacatcccctcagcatgtaagaccacttctgccccacccattgctgaatgggaccttcagatgctatcttctccataaagctttctatgactcaggccgcatcattttgtggtcaactactttcactattattgtgtttattctccctaaaccagccaagcaatcccatttggtaatcgaaagagcaagtagcactggcttggtcatttccttcacataaacctgactgaactggatatatggtaaggatttccatcttgtacataaacacttgtccacctccgtgattcacacaattccttttccactctatcccgggcccatgtctccatcctgcccctgatgctggctctgcaggcattttctacatggctgtctgtgtacatgccgtgtggttagtcttaaatcaagggctcctagagtgcataggacataccagtttatactttttctcatcaggaatatgtgtagctggaggattaagaaatacattttcatgagctagacaataacaaaaattaaatggcaaaagtccttgctgtccttagtagctagggagctttaaaaataaatacataagcacctgtttccgtttttttactcctacagaagcagggaaattgctggagaaaaaaaaaaaatgtaagtccatatattctgtactgtgtcccttggaagaataaaattttaaatgaggtcatcaggaagtgcttcctaaatgtgaagttcacagactgaactttgaagtttagcaatggacttcccctgagcaaggtcggcttatgctattgttagaccagcagctgagcctcccccaccacgtccttctacctcttgcaattaaagcacagcccaggcaggtttttcttaaacaggatttttctttatgttggtgcatatgctgctgttataatagcagggacattgtcctcattcccagctctttcctccaagtcaaaacatttagaatgacaccacccaaagaataagtcttacttgaggctttgcaacctctaacccttcccacctctaaccttttcccatctttatccccgttatcttgggtgctttcagttttcagagaccagctttccaacagggctaaataacactctatggtttctttccccatcagaaaccaaagaactccctgtttctcagcccttgccagcagaatctggtcacccctctgcccctcaaaattacacatcttatacaactagctcctgtccctgcaccattcctgtcccttgtccttgatcctggtttcccagtggaacaggaaaaaggttttacagagggttttttgccaccatctctttgttcctcaggagatgattaagttgtgcgttaagtggggaaaaagtacattaggtcaaccccaaaatctgccatcgataacatcagagtatgtccatggctttattagttttctagtgaatatttaagagaaaaaggtgctaggtcatagaacattgagaaggctatacatctccccttggctataggtgcaatacattccaagagggagagatggtagtcatgcctcctatttttttaggtgaggtatgtattttgtagttctgctgggacaaaaacagctttcagtcattgaaaaccaaaggcccagtggacatcaggaggagatggcctccctcactgcaagacgacctctgggtcaaagcctagtgaaactttggcttttcccagcaagcaggaagtgacatagacagtgtctttttagagacctcactttggagaatgctcagtgaggggcaaacatttgctggtgacattgtagttctgtcctttgctaagcttgagtgaaaatcttttggagaatcgacttttttctttcttttttttttttgatgtcccttagagagctcacaataaataaataaatcaaaacaaagctaaaagttcataaaaaagtctcaggcatggtccagattttcaggaaaccagcagtttctggaggaagtcccacccgctcctgaaaacagaaaaaataaacgagagagagtccaccagcaaacaggtgtttagtcttctctggagtcatattaagcaatttttacatttatggaaaagcaaacactccccccacatagggtgtggcccacactgggattgaaaatcagcctcgtgctctgcaccgtgttctggtggcttctgcagctgtcacaggaaggaatccacacccgtccccaaggacgcctgcacgggaacagatgcccgctcccttaccccttgggggaaggacagaggtatctgagggattcagccctgggcactcctctcagcttcctccttcacaggcggggccctgggaggccctgagtggggccactgcgttgctatgagatcagaacggtagccagttggtgggtgcagcaggctcacacctagcaagaacctacctctccctttttcctaagaagggaaaacaactctcaaggctcaccctggaggcccaaagggaaggctgtatgggtcccgtactgcagcttccaagcccacggtggttacgtctgagcaccctgtagggctctgctctgaaagtcttttcagctcccaatgagcattgttcaagccgatgaacatccccaaaacccttccactctcagggcctcggccccacccaccacctggcatctgtctttaaggatgtttataacagagttctgaaataaagttttggctttttcctggagttctaactctgcaggagaagcagtgaggtaggactttgaggaattcaggaagtggagaacagctcatggtgagaggaaaggctggggccgacttgggatgtggatgtgcaaaaggaaaggtgaaggctggatgggaggcagggtgggcacagttaaggtgggccctgggccaggatggcctcccaaccagagagtagccttcaacatctcagcctcgtactgcgtgccagacactctaccaagccctccacttaggtaataatcacgtcttccaccctcactctcaccctgtaagggagctcttagaataacgctgctctccacggctgggaaacccaggctctgatgggtgacatcacttgttcaaggtcactcagccagtaagggccaggctgggctgcaaactcaatggcccactgcagagctcacacaggtccatttcctactcgccatgatgttttccagggagagtttagagaagctctggagctgggaaaagagggcagctgttagagaacagccagcgagaatgggaagtcaggagacccagagctcaggaacagacttccttggcaggtggcgtagtcccatggcctctttagctctcagttttctcatctgcaagaggacaatgggccagattggtgcttctctgagagaaagtaccagtggttttgaccaagggactgacccgaacatggcatgtaagaggggtttgtttggggttggtccacaggatggttacagaggaaggaagaaagccaccaaggccaatttgaagatgtgcagaaacccagaagtgaggtgaggacggcagttctcaagtgcagagcttgaagtggtcactgctgggagggagacgtcataacaagacccaggaaacacttcaaaggaaaaactaacagcatttaaagattggctgcaatagtaacaataaaaaaagctaacatttactgaaggctataccagttatcccaccaagtgctcaactaccaatcctcacagcagccagctgcaggaattatcattgctcccactttatagataaggaaactgaagtttatagaggcagagaaacttgcccaaagtcacaaagctggcaactcccagagccaggatttgaacacagtctcagtacagagcctatggtttctttgtttttgagacaaagtctcactctgtcaccaaggctggagtgcagtggctcgatcttggctcactgcaacaattgcttggctccctggttcaagcgattctcctgcctcagcctcccgattagctgggattactggtgtgcaccaccatgccaggctaatttttgatttttttttttttttagtagatatggagtttcaccacattggccaggctggtctcgaactcctgacctcaagtgatctgcctgccttggcctcccagaatgctgggattacaggggtgagccactgggccccgccacctatgttcttaatccccgtgcaactctgcctcctatctatgtgccgaagaaaggaacaacactcccctccagacctctgcctttcacacccattggccttggatgcccttgtctccccccggttccgattcccttcaaattccagctcctttaaactttcacaccttactctgtccacccctccacacctgcctcgtgctgtccccccgcccccacatcccactccacccccacagttcaaacctgctcatggaagtcctggaatgtgttttctccctcatcctcagtcccacccatcacacagcctgcactggatcatccaagttacatgccaaatgtctgtacccctctgtgtccaagtgaggcccaccttcctctttgcttctgtcatctattttcctgtttcttgcatgcattgcactgtgttcattatgtctattgttgactgcttgttcatatttccaccttccctataggatcgtgagtgctttgaaggcagagagaggctgggccttagtacagtctgtttgcagcacatggcatatagtggatgctggagaaatgttgatgactgaattgacatgattgattgattggatcaaatgaatgaacacaagaataaatgaacatcgggatccaggaaaggggtccttctggcaccaaccctattctgtccccctaaacagtattacatttagtgtttatgataaaggctttcagcacctttttgaagagttggtaccactggtccactttctgccctgcccttaatccatccaattggtttagatactcacacatacattctttctttctctctccttcccttactggaccccagaccaaagccaaagaaataaatgtcccctctcctgctctgtttccacttagataaattaatcctcagcatgagaggaaccaaagccagcaccttagaagtcaggctcctgtcagctcggtttgctcacctgcacacagcccttgacttcctcctcctgtgtgggaggagcacagccctgactcagaggccactccaggcaatcctctcctcccaggcaatcctataaggaacccactggctgagctctggcctctcggcagagggggcaggcctgaagaaccctttccaagaccagctgagctctgctggtccgctgggccctgcctgcagagttattggcggagttattcttggtgctcattagcactgcacaggctgtggattattcctttattgctaagtggcccccagaacactgcagactcatccctccttttgaagtccttgtaaaagactccaagaagtcgattccaatctcttctctcccagcgtggactgcccctggagcagcactgggggtgtgtggctgttagagtggcagccaagggccagctccccagtgaaaggtgatattgtgcttctaccgcacacctgcacgttgggatggtgtttgccttccggcctgactgctctgttctcatcttccatggacatggaggtgggaggggaacttatgtgtcattcactccagggtcctgatggccacttgcgtagagcaaagaatgttggtagggcctgcagtctaggttctagcttcagtgagctctggggcctcgggaaaagggcttgatccctcagaacatctgcaggagtctgtgacccagaccacaccaagctttcagaaactgctctgctcttttactctcccactcctctccaccctctcaagctgtgctatatccacaatgcaccagcccaggctgactcaacttcccgcccactgctcccctttaagctacagcacccaccattttgccccaaactcgtctatcctacctgagctcctgcagccagactgggtcacccatgcccctcctctaaccaggaaagaaaggaaagaatccatcaggtcttttctgtgtcctgagattgcgcaggtattcacccgctccactcccatctccttgacctctttcttgccctgtttctcggcgacctaggcattgggtcctggtcagttttccacttttccatcactcccttaggacccctcaacctctctgctcatcaaaaaagtccaataaaggcattcatctctttgaatacttaattttacagtacctcttgcataactttcaagtttgaggtcaatacaaataaaatcatttaaaatgtctgatattaaacacttctgacccgaaaagcaaaaagccttttaacagatagttacattgatacaagagtccacaatgacaggtttcaaagatgaaagtaatggatcagtaaatagaacagtagagaaaacacagatgaaaggcaaataaccccacctgaaaaatccaaagctttcattttttttgaaaaaaaaattgtcaaaagcactgtattattttttaaaatcatgataaaaggagcatgcacctgtcaccctcccaaccccctctatacttgcccctttctatctttaagtgctgtggtctttgtggtgactttattggctcaagtttcctaggctggtactctcagagcctggtttcctaggagcagtactcataacgcaggtgcgatggacacctgtaggtgggtagtgccgcctgtgtgaaccaactcggctccaaagagcctaactccctatctgcatggttaatccaatcaaagcccaacctgctttcccacacaacagagttcaacagagcttttcattaaagcaacacatcatagcaaatacttaactagtacaaataaagcattttatttaaagcaaggtttaaagacatataaacattaagcatcagagctccattctttagttgctattcatcacagcttatgccagtccttgaattctgtctaacttgggtagtagccaattttctaactcaccttcaacttcttgggatgagtccttcccgggctccttgtttttctagaaatttcagtggcttgaccacacatcaaaacacacagggtggccaggcatggtggctcacacctgtaatcccagcactttgggaagccaaggcgggcagatcatctgaggtcaggggttcaaaactagcttgggcaacatggtgaaactcggtctgtactaaaaatacaaaagttagctgggtgtggtggtgtgcacctgtagtcccagctactcaggagactgaggcagaaaaatcgcttgaacctgggaggtggaggttgcagtgagccaagactgtgccactgtactccagcctgggcaacagagtgagactctgtctcaaaaatggggagcgaggggggagcccaccacacaacaaaacatatagggcttctcatctaaagtccgaataaaagaataatactatggctggtttgcactggaggaaagaaaagccctatcactgctaccttacttttctgatgttttgggttcctatgtcttatcccctcaggaaccaggggaaaaagaagattctgtgtcaagtgcaccctacatctgtccatgtcaccccccttcttccaatcagaacaactcatttcccccacttgactgctcccctaatctttgcaccttggagcaggatgtgtttattgattgatggatatttacttataattggtacctcttatgaaagccaacagtttttacatctggcttttaacacagactgtgctcttcaatagtcttagtgtagactgaaggtttttcaaattcacatttttagaaaagagttccattaaattccacatgaacaacagaaaaaaatttaaatgggacattacagtgatcaattatagattgtaggacatgactgacatgtagggctgggctgggaggaagagacaaagaccaaatgctcaaccaagggcagaggtgaggtgtagattacgggtcagaaccgtaacataacggatggagtcccaagtgaatccaggaagttggaggaagcaggtaagtacttgggttaggcattcacaggcctaggaagtcagctggggcaggaaggctcagacaaggcaaggggaccaggaagcttggacccgtcctgggaagatagagtccaagagggcagtcatcaccggtcccacactaaaagcatcaaagctctctaatcttctgtcttctctactctctctggtgtgttgtcaaggaaggaataggtacgtgggaaactagggagacaatcatagccctcgatgtctaccgtttctaattaatacagggcagaggaattagatcttttggtagagatctaaaattcctctgaagttctatcaagccccatctcctgatgattcagtgctagttctaaaataggaaaacttacttccaaaaccaacagatgtttgggctgtcatcattatgcctgtactaaactcacttgtgatttcactgtgaagtcctgtctgtgtaatttattaggaatcatttatgtaagattatgtagaaaatgttactagtcgtgagatatctatccatctcaatgatttgacttttacagcaatgcgatgggacgtgctgagggaccatttggtcaactttccacctaatggtggtgtgattagaactcctagcgggtgactcatagagagtgattagaagtaaaaaggttctggaaattcccccacactctttaaggcagcctttgtcaggtgtcttagtttcgtgaaaggtagactgatgttttccatctggtatacaaaacattttctaagtagttatagaaccttgaacaagccacttagctctctgggcctgagttttctcatcttataaaattaaatccatcaatagatcacatgatctctatggcttctttattctaaggatgtatggtaattttaaacacttggttagccatcttgaccaaactagcatcacaggtaagaaacaagctgcgaagaccaaaattcttctctggtttatacactctgggtcagaggcaaccactttctatgttgtttgggctacttttccctaaacacataatggctttgtctgcatcaaaggtcatcttctattttcctgcccattcatatgaccccataagaccttccagcatctcagtcagctgagcattttacctcctgcaaagcacatggtttccttcttctctttgcaggtcatatgaaatgctcacacagtacttactgtaatcacttcgtaaagttgattttcacatatcttcatccagaaaattatcccaactgttatatataactttattctaattgaataattccagttttcaagacacttttcagacacccattgtcttgtctgaatcctcatgacaacctcatttttttatagattatacagcattcctacttatatataatagttataactttttattcctaataaactttggcatgacgtttcatcagattctgatagtttcttttgtttttcattcattgcattcccatccaaatgtttactgaccctgggaaataaatccagtagaccaatgaagcatgaatccaactcacagagggtggttagaactaaaagctgaaatggacttccgtgatcacctgacccactcatccctgaccgtaggctcttgttcacagcggccaaaacgtgcatgtctctcaggggcttcctgcttgtggagcagaacctttgcttgcgggctccttgtgtctgtgagctcctcagaaccgctcttgagctcacagtgcctgatttgcacgtattggtttccgctccgttgcttattctggtcccctcatagaagcaatgttatcctttcttcaaacccgtcctttcccagagagctttgcagccacttttaccgggtttaatgagtcagctgcagagtttatgatccggtactaggccttctgggaagcagctttcctgggcttacgtcaggctgttaaagaccctcctggccttcctgccgacatttgctcggctactttttcctcctaattaatgcctctatcttatcagagtggcccattctcaaatgcagtaaatgagtccttgatgttctatgttttattttggaaacttcccactctgctcatatctgaattggtatttgctcactgttgcgcagtggtttgcctaccaggtatcccttacatcaaatcctgtccgccgcttagaaaaaccacagctaaaagttgtttggcaactcttggtgttcacagggttttcccactgagggtcagggactgcacagcggttgagagcttggactccggggtcagctggaactgcttctaacacaaggaccgctcttaacaaagaactgaggcttcctaaacttcagtttctgggtctgtaaaataataggacccacttcctataaatgttgagaagattaatgggaatgatgcatgtaaaatgctcggtacatgataaaagcccagtgattactcattatcccactcaatgcttggggaaaggcaaagcagctaatattaccccagattatagataagaatactgggttcaaaagagatcacttggcttacccttgtctatactgcacctgcatgaatcatggacctgaacccaggtcacggccacctcccagacccttgctctctctgcttccccagcagcgcctctcagagtgaacacctatgtccatgacagtgattcccaaagtgttgtccccaaaccagcagcatcagcatcacctgggaacttgttagacatgttagggtctcaccccagacctaatgaatcagaaactctgagaatgagggcccacaatctgggttttagcaagatctccagaagattctgatgcacacccgtttgagaaccaaagctccatggaatactaatttgcctttggatctagccctgccgcccactcgagatggatggtccacaaatctcaatgtgattcctaacatgagttaagctgtccaaccctgtgacgttatcaggggatttgtgattaatacatagtgacaccatttaactgtggcagaggttcttacccttggaagctcattagaatcatctagggaacttataacagaaaggcccaccccagaccagttcagatctaaagacagcctggactgacaaccacagaactacagagctgccacccagactccatggaactctgctaggctggtaatatttttaattcattaagggtttatgtgtttaacaacccaggtccagtaaattggctcctttcctccctatgaaccttggacactctggctggtcaagcctccacctcatgcccacttgtttgcacagctactaacgtttatgaagtatttgcttaatgtacctaagctccactgcccagagctatcccacttgatctgaaaacgtcatgatgaggtatggtaagcattaaacattagttcaccatagttctatttttttctaataacttaaacggcttcagggaacattttgtgtgtgtgtgtgtgtgtgtgtgtgtgtgtgtgtgtgtgtacgccttaagagactggatctcactctgttgcccaggctggagtgcagtggcatgatcgtagtttactctaacattgaactcctggcccaagtaatcctcctgcctcagcctcccaaagcactgggatcacaggcatgaaccaccatacccaccctaaggaatatagttttttgagcatgttggagtactcagaactctcagctcagctcttggcccacttgggtctcaaccatttagagtactgattaccaaaggaagagcacaaaggaagaaaatcagcttttctcctcttcctcagtgtgggagaggggatttggcttgggctcaaagccatagtgatgctttctgctccaccccctttcaattaaagatcaccagcttcccaaaaacaggccaccctcattttccaacctggctcaggcagggaagctgagaagcttggggctgcccattcacccagtcaccaggcagatagacaggtaagagaactgggaccttcaggcctgccctcgggaggactggccaccaggctttacagcccagagcagagaaacactgtggccgttgcaaaactccttgagtgaagtgaccctaccctctactaaaaacgtggcccagtggattccagttactaacaaagctcgttgagaagatgcaagaaaggaatggaacaatggctaggacctccccgcggcagcacttcccccaagagaatagaggagctgatgggcacgtcacatttttctcaatgctggatgacattccatcaccttcctcatcttcatggaatttttttttttttttttgagacaaggtctccctttatcacccaaactggagtgcagtggtgcaatcatgcctcactgcagtcttgacctcccaggctcaagggatcttcccacctcagactcctgagtagctgggactacgggtacataccaccacacccggataatttttgtaattttttagagataaggtttcaccacattgcccaagctggtctcaaattcatggcctcaaacagtgcttcgggttcagcctcccaaagtgctgagattacaggcatgagtcaccgcacccagctggaatgcattctttgaattttaaaagactgtaaatgagagatttgttcagagaagagggtgtttcctatgtacaaaaccaaagaccagcttttattttttattaggtattatagtcacaaaggtatgaaagggtatatagtaataagtctaagaaatttttatgaagtttttgaagtaaaaagcatttttataaaattttaaataaaaattttaataaaattactcaattgttaaatgtaacttaaaattactttttcataaaattactcattttataaaacacatttttcctactttgaaaagtagtacatgcttaatacagaattggaaaagacaaaaatacagaaagaattgcatttaactccttttttttttttttttggtagagacgaaatctcgctctgtcacccaggctggagttctgtggcgagatctcggctcaccgcaacctccgcctcccgggtgcacaccattctgctgcctcagcctccccagtagctgggactacaggtgccagccaccacacacagctaatttttatatttttagtagacatgggatttcactctattagcagccaggatggtcttgatctcctgacctcgtgatccgcccaagtcaaaacttcagattttgccaaacaccttgaaagtatgagtagatagaggaagactcgtccttttgtctgcctccttttcatcttcctaatacctaacacctctctaatctttctagaaatgggtaagaaaagcttccataaagaaaggtggagagaattgaagcatggtagtatttttgcaggagaaattataaggtttgaaatttatggaggcctcaggacatacccaatgacctaaggtctaggccagagatggtaagtaggattttttttttcttttctcttttttttttttttttttttgagatgcggtctcactctgtcacccaggctggaatacagtggtgtcatctcggttcactgcaacctctacctcccagattcaagcgattctcctacctcagcctcccgagtagctgggattacaggcactcgccaccatgcctagctaattttttttgtatttttagtagagatgggggggggtggtctcaccatgttggccaagctggtctcgaactcttgacctcaaatgatccaccagccttggcctcccaaagtgctggggttacaggtgtgagccatcacacccagctgataagtagctttaaacacaagtgaggattctattccatcaggaatggctgctttatgcagaagaactctaagattacatcaggctaagggtgacagaatgctgtaatccattagccttatctgtcatggtcatggcagcagcaagaggcagtctgagtgccatacactgcagtaatctaggggtgagctgaatagtgaatggagatttgcctaaaagaaacttctggagtcagtgctccaaaactctgaatcaaccaaatgaatgaattgagccaaatagaaatgcatgactattcaatatttattatgtagagaggcttagaaagggcaggcacctggagttcctccaccctcctcacccaatcacaactgcccttactgccctaccagtccagccacgtgccctttcccacccctctcaatgacccttgggctccccagtgaagctccctgagggcaggggacatagctctttggggccataaaggcattagctcacccttctcagccccttacacatttactgggtacttagatcaggccaagtgtggtggctcatgcctgtaatcccagcactttgggaggccaaggtgggtggatcacctgaggtcaggagttcaagaccagcctgatcaacatcgtgaaaccccatctctacgaaaaatacaaaaaattagccaggcatggtggcacgtgcctgtaatcccagctactcgggaggctgaagcaggacaatcacttgaacctgggaggcagaggttgcaatgagcaaagattgcaccattgcactccaccctgggcaacagagtgacatttggtctcaaaaaaaaaaaaaaaaaaaaaaagaaatcttagcaggattacatctgtaaccccggcactttgggaggccgagggaggtgaatcacatgaggccaggtgttcaagaccagcctgaccaacatggtgaaaccctgtctctaccaaaacatacaaaagttagccaagtgtggtagcatgcgcctgtaatcccaactactcaggaagctgaggcatgcgaatcacttgatcctgggagatggaggttgcagtgagccaagatcctgtcactgcactccagcctgggcaagagagtgagactcttgtctcaaaaaaaaaaaaaaaaaatcttgccccttccctcaaggggctcacagcctgtgggagagggaaaatagatataattatatttaaagaattataaaacaaattgtataaatttaaaattataattaaatcattgtaataatttagatcagaaaaaataaaggcaagtaagtgccaggcatggggctaagcattgtactcacccactgtattccatgttgtcttcacaacggccttatgaggtttgctagtattgcccccattttaccaagaaactgaggcttagagcattcaagctagccaagggctaagccatgactcaaacccaggtgtgtctgactacggctgttactaagaggggaagggaatcatctgaacttgggtttcactcagaaaggggcatggttttgttaactttgaaaacactaccaccctaccctctagcactggcagaggggagctggacgcctggacagctggggagggggaggacggcagccagaagcaacagcctctgcagtcccagcagctgggacctcatgcctcaaccaaggatcagcactgggtattggctcagagtagtgaaggctgctgggggcctgtgtttcctgcatttcctatgtttatgctgccagggtgggttttatgtgaccagagagaagcagggaaaattgctgagaagtcagagatctgccagggaatcagtgatgagccccaggaaatgctttttgtctaaaaatatgccatttccgaaaggaaaacagtcattagagggtgtgagaagccctcgtggtgaggaaagcaaaactgtaagactcaatgggaggccagtggaacaacagagaagcccattacccacccacttcctgtgcgattaacaagacgtttggaggatgaaaggcaagcagacaaaatgacaacccacagacacaactccaggggaaatgtctggggacagcataggaagacgcagcagggaggtaggaggagaaaaaactgctccagttaatatctttctggcatcggctggttcagctatagatttggctaaaattacctgtgtacttactagacgccaggcagttagggggctttgcttaaatcactctcctgcattcttcacacagcacttgatgaaggaggaaagtgagtcttggaggttaagtgacttgtccaaggttacacagctgggaagggagaaagacatggctccatcccaggttccctgatcccaaatctccccctcattgcaatgtaccaagctgcctccaataagatcaagagtcttgacgatccccaagctagtccactcagtccagttttcctgccagactatagctgttttgcattccttatcttgtgttgaagcaaaccaggtaaggaaactccccaccttttttattaatgactcattttcagccaaaacacattgcagccacatcaggtacatgtgcccactcagcaaagacgtactgtgagaaagtaacatctaagttacttggtccaaagcccttctacatcaatcatcaagcaaccaaccctcctttgctgaaatctccaggggactaggccctttggtgggggaagaggggacggtttcttcaggggcatctcacccctgggaagtgcatcctctctgctgggggcagtaaaaaggctttacgtgggccactgtggctcatgcctgtaatcccaacactttgggggcccaagcagggtgattcacttgagcccagaagtttgagaccagccgggcaatataaagagaccccatctccacaaaaaaatacaaaatttagctgggcttgatggtcccacctgtggtcccacttacttacttgggaggctgaggcaagaggatcagaaggtcaaggctgcagtgagccatgattgcaccactgcactcaaacctaggcaacagagctagaccctgtctcaggaaaaaaaaaaaaaaaaaaagaggcttttcacgaatttcacattccaccctccaccatctttgatgtaggtaatttatcagcattttgtagaggaggaaacaaggtctcccctacgttgagcccaaatcagtttgaccttggggaacacagtgagcatccctgcgtggaggtacgcaggatccctgagggtgacaaccttcgaggtcgttccctggcctctctgcccagggtgagcatcctcagagacctcagctctcctctcagcatgcaattctcctccactcatggccctcccagctgcccctggcttctcaaagctactcctgcatggaggcaagtacaactagccaatgagcctcctgacctgccccatcccagagagcaactgctgctaggtcctcgtggattctgggcctctgtccagtgaccaaaggagaggacacatctccagctaccatccctccacctattttctaaggaaaccaatgagttctcaaatgccacctgtggtggccccttctcagccctcaccctgagaggccttccccaagtcagaatttcccacaggggaagttactgtgggcttctctctccttcccccacctcatgtggtcaggaagtccagctggctttttctcctgtctgtctctcaaatgtgtcccctggctcccctccattgccactgccctcttcagtgtctggcctgtgctcccacaacaacaggcttgaagctggctgccctctcccctccatcttccacaccataatccaggtgatccttctaagaggcaaaggtaatcatgtttcttccctgctcaagatctgtcaaaagattctgtggcctccatagaaaaggcagacagcccggccacccaggcccttcaccatctgcctcttgaccatctccagtgtcctgcagtgtcctgaatataccttttccctgcctctgcccaacacactcccgttctttcaacacccaactcaactattaccttctctccatcagatcttccctgaaccaccccccagaaacaaaggtgactgtcccctctggttccccacagccaccccaccacttacccctgtgctcaggcactcctggagcactgcattgcaaccattggctcacacatctgtctctaccagcaaaccactaaaccgtgaataagtggcttcctgagccaggccttgtgcaatgaatggatttgcaacaaagggagccactgagagattacatgagccaagtcatggcaattagtccccatggaagcagcagctcagggcagggtccagacttgggcagacttaactatgcaggacattccattaacaataagcaccactctgagcatttcctatgtgccaggaactacgtgaagctctagattctgtcattagaaaggactgagttaattaatggaaagcaggtagcacagtgcctgacacagtataagcactgaacacacgttagcaagaaaagtgacagatcatttacgggatggttcttacaccaggcagcgtgccaagcacagtccatacattgtctcactcttcatgacaaccttaggaagagagcagtcctgtttgtccccattttacagaaaaagatactgagtcttggctaggctaagcgatctgctcaaagtcttcagctcggaagtagtggatttggtcttccatccccagtccatctgctctggcacccaggttctcagagtctctggtacagcagggtgccaaagtccagttgagaagaaagcaggccaggtttttcaggcacagcctgatgaaatatccaggggaaacacatgggactagcaggcttctttttctgtcccatttagtctaatatcacatttaacacaccatcctctaagggacagaagtcacaggaagccatgcagtctggttcctcctgatttctcctgtgtgccacatagttatgcttccttctgggaaggtgtgagttggagtcagtcccctcctgcatgcagctggggaaactgaagccaacaccattttagttactcacatggagctaagacaatcatacaaaactcaggcatggggtcagcccaccagcagtgtggacctcaccctgccggctgcgttccaaatgacagtggcatggtgccacagaaaaaacgtgaggtgtgcagctggacccccagaagccctgactctgccaggtgctagctctgggacatcttgggcaagccatttggcccgagtctttttccccacctatgaaacaagccagagagctcattcctcacaaggctgtggtcagaatgtgcctctgtacttgagaagctgtaaaatgttcctcagttgctgcccttagtagggagctcagaggaaagctagttttgcctgtgcgtttctgtcggattgccctgtacccagggcacgtcctgctctctgacaacaatcagggtcattctaactcaaatcaaacaacctggtctcagaactggtcttgcaccaccatggtgttaagctgccttcccacttcatccatgtttgctaagccgggtgggatctttcccctacaaactcctataggcggccacatttttcttttaaagatattttcccttcgactatagtccacaggcatcagaggatgaagctagcaggttgtctgtatgtagactttcttccagggaggaaagcagctttgctatctcctacctttttaaacaaaatctgccttgattttgtctgccaagatatgtctttggatggggtaaagcacttctgagggaagctacgtgagccacgttgtagctggagagagaattgaaagaggaagccgagctgagaccaccacagttccggggctggagcccagctttgcagaatgcctgcatagcagtttgaccccaacagatgaggctgttagggacagaaatgaaaagcactgagagaggggaagtgtgggtggagcttatggacattctatgagccaaagcaattttgaagtctaatcatttggagggaaaatatttgtcatgtcattttaaagggtacaaaacaacacagaggaaaatgaaagggttagaaaataattggagcgcttctccctttacccttgtagaagacagctctccggttttcagctccactcattctgtcctccattggaagctcccccagtgtcttagagatggatgatgtaaaagttagaaaatgcctgtgtttcctgcttgccttaatttaagaaactgcttgactgaaaagtgactgaatttcatttataggataaaatacttgatctccagggaagccaggcctcaatagatggatttttaccatgtcacttccccctagacctgtgggaactgacctgagtcttggggaaaatttgagccacatggcagtccgcaggtgcccacaagtccctctatgatctgagtacctgtcttcccagtctcatctcacctccctcctctccagccagggggacgtgcaccctatacccaccccatgcacacctgtcccaggctctggctttcagctcctaccccagaaatgcccacctccccccatagctcagtgtacccaagcttggcccatcctaaagccatctctgataactccaccacaacctgtcttggtccaagctggaagtgatgagtgaactctcagaataaataatagtagctcacatttcttgagctaagagccagacacttcacaaactcactctcacatttctttttttgttagtttgtttgtttgggagacagtcacgctccatcacccaggctggaatgcagtggcataatctcggctcactgtaacctccacctcctgggttcaagcttttctctggcctcagcctcccaagtacctgggactacaggcgcacaccaccatacctggataatttttgtatttttagtagagatagggtttcaacatgttggccaggctggtctcgagctcctgacctcaagtgatccaccgatcttggcctcccgaaagtgctaggattataagcatgagccactgcgcccagccagctcactctcaacttaatcctggaacaaccatataatataggtattactatgatttatccccatttcatagaggaggtaattaggacttagggaggttatgcaagttacccaaggtcatatagttgataagtatcagagccaagagttgaactcataccacctttcccaagagcaccttgagctactagaactactgcacgcttgttttgtaacatttacagaactatagttcagtgggcaaaggccatgcatgattcttcacacctcccacaatactgagccaccagcaatcaatgcatgaataaatgaatgaatgagaaaatacccacagactagagagttaaggcaacttttacaaacattaaaaggatgccatgtcaacaaaaacacattaagcaccaaccgtgtggtccacattctgacgggctctaggcctcggagttgaagccagggtccactggactttcagatgttcaatgaattcccactttccaacctagaagacagtcttctatcgaggactgcttttcagtctcctagctgctgctttgttcattattgctcagcaaacagtgtgatttatgcatgagacacacaccttaagatgcaatgtagttcttccaactgtcataactactaaattgaattgaatggcaatctactggtagctcaacttctgcaggaaaaaataatcagcctgaaaataatcaatgcaatagcactgtagtgacactgtcgaaaactgtacaaacagtaggaaaggtaatgttgtgactgtcatttattaatgcagagttctagtgttcctaagataaagatcattgaaatgtgaagcgcatttgtccaaacagaatttctaataaatacctttgattgttcctttgtctctatcctttactaaagtcaggcaattagcagtgtttgcaatattacaggcaaatgaaattgggtctctgcctggtccgcctacagaggacttctatagcaataaaaataatcaactggattcttcgttttctgctgggtccatgtgatttccttaataatgtcaatgcagattgtaatttgtaggaaaaaaatactgcttgcatcaggcatgaaatttaagaccacagaaatgccatcttttcttacagaattgttttgttacaagggtatgtggcttggacgattagaagagacctcaggtgtggtgcccaattcatctttcatagaccaatattttgtttcccaaatgtgccttgtacatttctccaataccttcactctctagctgttggtgagggaaaggacaaaaattagagtcataaaatcaccattaaccccccagcttgaccactcactagctagtcatttaggctccgtgggcctcagtttgcttctctgtaaaatgaggcagaaaactatgaaagtgcctccaatctaagttccccaggctcctaagacaaggcctggcccatagaaggcacacagtacatgttgcttgagcaaataagtcagtttcttgtatggtatctggcatgtgatcagcataattaatggttgcttaaaaattaacagtctgttccccaccagtaacattcatcttcttcctcttggcctggtgaaatcttacgcattcttcaaagctaacatcacattctgctttgtctgagaaaccttcccaagaccacaccaactagaagtgatctatctccctaaactcctaagacatgtgttgtccataagcttcattcattcattcattcattttatgtatttatttattttcgaaatggagtctcgctttgtcacccaagctggagagcagcggcgcaatctcggctcactgcaacctctgtttcctgggttcaagcgattctcctgccttggcgtctcaagtagatgggattacaggtgcctgccaccacacctgactaatttttgtatttttagtagagatgaggtttcactatgttggccaggttgatcccaaacccctgacctcaagtgatccgcccccaccttggcctcccaaagtgctgggattacaggtgtgagccactgtgcccggcctataagtttcatttaaaacaagcttgtccaaccttcagcccaggactgccttggtccagaagctttgaatgcagcccaacacaaattcataaactttcttgaaacgttatgagattttttcatgattttctttttgtagcttatcagctaccgttagttttagtgtattttatgtgtggcccaagacaattcttcttccaatgtggcccagggaagccaaaagattggactgtcctgatttagaacttgggactcatggccttgtactgtaggttcccctctcaaaggatttcttagctgttcagttatgtcacaagcttctcggaggcaggtccagagacttcggttcctggtgatttaaacagcccctagtcaagagcatggcacacaacagatgttccataagcatccacagattgactgggggaaatgtccaattcccatggagatggtcagatgccctggagaggccacagtgattacaagccctacagccctccatgggatacactgggccaaagacactggtacagaagacttgtcttctgtagaaagtttgctccatttcgctaaatatcttcccatttgtaaggaaactgatatcctcatgacttgtggtgggagggaaattgtacaacctcttcaggggacagagtgacaatgactctcaaagtttaaatgcacatacaattttaaaagattaaaaaaaccataaatgtcctttgacacagtaattgcacttataaatgtttatctcacagactgacctacacatgtatgagaaaagtcattgagaagattcacttttgggttatctgtagtagcaaaagactggaaacaaatgtctatcaacaggtaactggataaataaatcaggatatatccatgtacaagaacactatgcagccttttaaaagactgaagttggccaggcacgacagctcacacctgtactcccagcactttgagactgaggcaggcagatcacctgaggtcaggagttcaagaccagcttggccaacatggcgaaaccccatctcttctaaaaaaatataaaaattagccaggcctggcacctgtaaccccagctactcgagaggctgaggcagggagaattgcttgaacctgggaggcagaggttacggtgagccgagatcgggccactgcactccagcctggccagcagagcgagactccatctctaaataaatagatagaataaagtagaaatgtatgtacttgtgtagaatattctccaagatataagtgaaaagacaaggtgccaaccagtatgcatggcttcatccagcttatgtaaaaagggagatgtacacatgtgcacttgtatgtgcctatcctgtctccagaacaatagcaacggctgacttggggcagcagggaaagagcctggggtcagaagcaggaggagacttgtcactatattcggtattctcattcatatggactaaattttttagcaagtgcatgcattatctttcttttaaattttaactttatttttaatcagatgatgaagtcacatgcacaaaatcatgaaaggtacaaaatggtacatggtaaaacgtcttcctcccagccctgtccccagccacccagtttccagcctagagacacctaataggaaccctttcctctgccctttttagagacagtccatgcattgcagagattttcttttaaaataatcacaatgaagtgaactgggagaaagcttccattgacctcaccaccctctccagctcatgctctgttctccagagccaagctttcacacagggttgtctacgtgtatggagtggtgaaaaagagttccttgggctttggagtcagacaaacctgagtcttcaatagttgacctaacctctctaggtcttatggcttcatccataaaatggacaattcatacctggctcttaggttgttataagggtaagaaacagtccatgaaaagcatatgggtcacttagccacagccactctaaatttctgggcaccagcaccctccatggctgggaccaatcctcactgagcagtgttcctgggcacagccagagaccccaagacccctggatgtccccctaatcccctgagaagtacatcctgtatttctgaggtaacattttacggtaaacttctacagcaagcaaaaaaccttaaggagctcatgatcaagaccagtcctccctgggtttgggggcttttgaaagggcccaggtttagacatcaggaaacctgtatttgaggcctgactctgccattaactgagcctcctggagtgattcgcttctcatcccaagccttgcacagactgggtggtgattcctgaggcccagaacctggccagccttatagcgtcagtcagggatgtgttagtggagcccttcactcagcgaaagcatagctggctgagtctctagacagagagtgtgttgttcaggctaaactatctccctaccatggacataaaatgacatgggtatagaacacactaaattcttcaaggtaacaaatttaaattcttcaaaaagggagtggctatttaaaatatctctagttcatttttagcattactgaaattcaatgtaaatcacagtggccatatagagttgacatataaccctccctctgtcaaacaccaggttactaaagtcacagggattaagaaatgacccttggtaacaaaagttaccaatgtagaccagttatgtggcaggtggggagagctattctaaggttccttctaactccaaggggctcagatcggatatttggcaagtaagcaattggcacactgaccctgcagtttacaaagtgattcctacgatcctgagggtagaagaagattttgctcctggcagaggttttgcaggatgtgggtggggttggagaggaggctgctgtgagggtagaagggggtaacctctgtcccttcaccatgtttatcttgccatatcaccatccagcattcacagtccattgcccaaaataaaccacagatacaaagtagaaagatacttcatctattttctaagtaaatctaggaagttaaagatcacagctgtctccaagaccatgcccgttgagtaaaaatatctcttggcaagttgtcctacatcaattttttttaatgagacctcttttttcctacctagtattgataatttaatagctctgcaaaaagtgcttctgaagcaagaaaacttagtattttctaatttattctctgggagatttgactgctaatcaaatatcttaattccctccatacttaaagggggcattttaaagtgactttcccagccgttccaaattacttcttgaggtttattgtttgcttttaaatttatgtccaacatgggctctcttcttggggactgagctgtttcagaaaatgatggaaatttggggtgaatctaccaacagtgttcagtctttccaaaaagacagttcattaaaagctgaaaggtttgaccctctggctctatctttttggcctaacatcctaacacaggtgtggacagggtcaagcatgcaggcctgtctctgatacctggttatagcaaggctgtccttggcatttcacctccacctcttctgcatcatacattgtttattttcacctcattattccattcctactccatttggtgataatccactgtgtgctaggcacccttctgggcaccagcatatagaaaaatgccttagcattcgtccctgccctcaaaagggccttgggatcaagaagaagtggacacgagcacatctgactaaacacaacatcatggagggtgtatccaggatatgagcaaaatgctcagagacaactctttttatttacgtcacaaatgcttctacagcacctactatgggccaggcatcatgctaataagggctttaccaatattaattcatttaatcctcacagcaacccactgagatgaatactattcttatcctttggtactgtggggaaactgaggcacagagcagggaaataacttgcccaaggttacacagccagtcagctccaggtggggctcatggggcatgggccctggagcccacacactctgactccagggcccacactcctaccgtcgggccacactgcctcacaacacactttctcgccaaattcagagacacataactaaaaccacttacaaatggagtatgccaactgagaatgctacagaacacattgaggaagtgacatttacactaaaatctgaatgatgaacaggagttaactagactaacaggggaggaagtgggttgcaagccgtggttctgggtggggaaggagtgcaatgtgttccaggaactcaccaaggccaccagaaaaggagggtggaaaagaaggaagaaaacagctcaagaagaggctaaaacatgagcagcaaccatagcattcagcaccctcactacaggccaagggtcattccgtagttgggttccgcaagaatgataggaacctaacagctttggcaggggtaatagcatcagttatgcatcatccactgatcacttgggctgacatgtagaatgcattggaagaggacggcggggttaagcagggagaccccgggggaggcagcagcaaggagagagttgatactcccttggatgggctggaggtggataaaaatggattcaagtgagttttaggagacagtatcaacatgttatgctgcatgtttgcttatgatgggtgaggagagggaggagtcaatggtggctctgaagattctaggtattcacagggtcgggaagcacttgtttgtcacaggtttgttgggtgccggaggggaagaaagtcccgtgcaacagccacatgcaatgttcatcactcccctggctctcaggctgcttcagggctccagaccctcgcacgtgctgatctcgggctaagggctgtttccttccttcgactgaagacctttcatactgtaagattcagcttgaaagtctctccctccatacacatcacacgctgacactggaccaggcacttatttggatctcacaacatctctgcaaagaagatattgctatgcctgttttacagactactgagaatcagagaagttccaaagctgccctgtcatttgtggcataagcgagagagcccactgacatcccagcgctgccttcccagccacactgagggagaggtgggcttggcccttgaggccccttcttctcaaaggcattgaggttactttccatgtagattacggttgagaacaatgagaacttctggagcacattctgtgggctgtgtgaggacgtcggcaagggaacagactaagatggctagaaaatgcccctctttcatctctagaattcatctagtgaagcacttcccaagcctcaggtatccagggtctccctgttcttcctagaagtaggttccagtgaacagaaagcccacagcataactaagaaaaagctgtgtgtctctaccatgctggtgaatccatgctgctccttgacctgccactccttgacatggacggctctattctaacagtgccactctctcatgtcactgtccctctgcctggttcctcctcctctcttatctgcctggagagtcccactccttggatgagtctcacccagagcatccctcccccatctcctcaggctgggtgtagggtgaccagctgtcctagtatgactgggcctaacggatgttctggtttgtgggactttgggtagtatatcagctcaatcccatgcaaaccaggatggttggtcaccctagttaggcatgtggattgcccctgcttatggcaagcccagcaagtatccaagctgtcttgcactcattggttctctcctctgctctttccccaaaagtagttatgaccctctgagatgaagccctgtgccttgttcatcactgtattctcaacatctaggcctggtacagaccaggttgtgtggtcaatgtttgtgcaatgaacgtacgagcccagactcagcatatttaggagatggctcctaccacccagcaattagagaggggaggcaacgcaggcggaggacatctaccaatgcagcacatgagcaagacacagggcctgagaataactgaaccctggcccacagatgccaaagaactggttttgcttgtttgtttcatttgccagtcagaagcgtgggggataatttagtggaatcccttcacagataggttttaacttgaaataattcaactataggtgcttggcaaacaatcaaatagtgaacaggcccaagagagaaggaaataatttagatggcagatggcagttggctccactagaagtgccacctggtgagcacgggcccacagtgggtgtgaggcgggacaacgggggcacacttccctcctgcgtgtgtacctcacactgtgctaggatgattatggttctggcatacaaagtcacttatttttcatgcaacctggtccctaaacttaagaaaatgcctttatctggtaatcaactgggaaaaacattggctgaaccccatactttcaagtacttatgagattttcaaaggtggttcttacacactaacttaagagtccccagatggtgagtcccagctgcgccacttcataactaaatgatcttaagtagctaatgacttaaccttgtagagcttccattctcatgggcaaatgaaggataacatctctccctcatgggctcacagtgaggattaaacgaagggaaggtatgcacaacagttagcagactgcttggtacaaagtaagcgctcaataaaccaaagctattatttttagtggtggtaatagcactgcttttctttggcatttaaagctgtgtagctaatctgggcattccaccaggttgctgaagcaatagacggccccttaataaatagcttccagacagcatgtcatttaaaactgaatgcatttttgcttctcagttgttggacatattatttactacctgatggtcttgctctggcccaaacaagctaattattgcaattccagagtacatgccatcatccagaaatagctttctgtagagtggggactgaaattaacaggcaattagcaagtttaaaaggatttcctttccccatccccacccctggcacctctaacacattaaagctcatcacagcagttattttaaggaaattcagcagaggtacttgcagggcccgaggtcatcttgtgttggccctggatagatcatgaaatcctgttgccaaggagcatggtgggaatgtggcaggaaggctaaagtcaggaaagccacctcagccactaagtagccatatggcctggggcaagccactactcaactctgagcttcagtttcttcatcagtaaggaagtggccaggcacggtagctcacacctgtaatccagaacttggggaggccaaggccagtggatccctcgaggtcaggaggtcaaggccagtctggccaacatggaaaacccccatttctactaaaactacaaaaattagccgggcatggtagcaaacacctgtaatcccagctactcaggaggctgaggcaggaggatggcttgaatccaagaggcggaggtggcatgagccaagatcatgccactactcccaagcctgggcaatggagactccatctcaataaataaagttaagaagttagactgctcaattcctgagactccttccaacccagcaatctgacgtccacactggggctgaccactgaactacatggaggcacatgcctcagtttccttatctacgccattcatctaacagctatgtatcaatccctgttaggcactaggcactgtgctaggtgtttggaagagtcattctcaggcctatcctatctcagcaacctgtaggaatgtcctcacctccagagttagcccccttggaaggaaaatatttagtcccacattcatttctttcctatatgaacctggaatcactgggggcagcctctctgaactgccccattttctgaaaagtgtccgaagtacagcccaggcatggggcattcagcacaaagggcccaagtcttgagagggagtagtttaggagtgaagaaatcccagcctcccactatccagtcttaaggtaaaaagaattgtacagatgagcacctttcttccccttggtatttccctttggcatgacccaaagaccactcccataaagggccataccctagtgttttctgacaatctagggatgaattccctattacatctcaggatgggagaaaaacaggatttgggagttctggtttaatcactaaatgaacttaatcttgacttctggttttgtcatctgcaaaaggcaagtggtcctgatggctacttaataaagctgtctgtgagccaaatgagagtgtgcatctgcaagtgcattgtaaactgcaaaaataattgtggttctctctgggccgccttctacttcacaacaatactgagctgaccttacagataagctgaacagatggttgcctccatggtagtatttgaatagcaccagaaagtttcacctctccaaagcatctttctgctggcctgaaattcaaccattagggattcattttgttgcgggggcgggggggcggcttacaagcttcccagggttcaggagcactctattaaatagctgttgtaatcacccagcaggttcttcctattcgctgcatagacaaaaccagttcaccaagaccacgctattgtagtagagtttaacacaaagccagctacgcagaagacagttattactccaatcagtctccctgaatgctcagcggttagggtttttcaaggatagtttgatgtacaggggattagggaatgggtgctgcagattggttggggatgaaatcatagggttatggaaaacggtccttgtgcactcagttggcctctgggtgggggccccaagactggctgagtcatgagcctcaagtccggatgtagtcagtctaaaaaacaactcaaaagatccatcttaggctctacaatagtgatgttatctacagaagcaattaggcaagtcacaaatcttgtgacctctggccacatgacccctgagcagtaagggatgatagaaaatttgcctatatcttagcagaattcaggcccctttcataatcctgaccttctggcctttcactggttttgcaaaagtggttgagtttggggagtggctattatccttgctgtaaggttcaactataaactaaattcctcccaaagttagcttggcctacacctaggaatgaccaaggacagcttggaggttaaaagcaagatggagtcaactatgtcagatttctctgtcatcatttcacaaaggcagtttcactgtgacctgctaatactgtggggaaaagaaaaaaacagtactcattgggccttaggcatattctaggttctttgcaatacttaattttgtttttaatctcaaaccagtctgagacaggatttactattcagcacatttttgaaactgggaaaatgagtctcgagatgtttaggaaacttgctaaaaggtcacatgagtgacagggttaggacttaaacatgcgtggggtcgggaagatggcaggcacagggtatcaggccaggggagatgccctagagccagcgacactcagcctacttaacttcttctaagagccggtcacctccataaggacagccagagcttctgccaggaaagaaagaacgttgatgctattcgttttccaaaagagcgtatctcctccggctaatctgaacatatttcttacagggctcttgaatgcccctttgtagctgagtgggtgaaacggcatcaccaacatttggccctgctgctccactgagagccggcgccgttcgcgggataattatcctgtagtcttctcacctccggagagaatgcaaggcgccctccttccaaatctggcccgtcacgccagcaccacttatttttagaagctgttttcccctcatagtttcacaacctctaatctgccactctccagtgaatataaattaaaacacaacaatgaaatatggagaaatgttttctcccgtcatctccagctctctgaacccggggctgggagtgggtggagcgcctgatttgtctcccaggcgacacggaaagggaggcagtgagacgtgctacctggtcgcttccggctctgatgaaccactgccagccagttattagggaggagggcagcacttgcaaaaatttcaggtctctgagagcccagagcctgagaagtgtggaggatactaattacacccaggtggaagcctggccacagctcacctcctaaggaaagagcgagcccattaaatagccaggaaggaatggagttcacaggggattagatttctagattatagcctcagagtcattctcaaatttatctcctaaaatctggaggtcaaggaacaaaagcacccttgaagaacaggtaaagggagcctggtctgaattcttagctcctgacagccaggccgcaagaggacagacatcccgtgcacacacgcccgccccatcgccaaagaaagctctagagctagacacttagactggcctttcatgaaatgacagcacctgccaaacacaatagggaaatagttagtccagccaggcagtgccatagagtggtcagagcatgggaatttcaagcagacagcagcaggctttaatcctcactctactctccccagatgtgtcaccaaggcaagtcactcagcttctctgaggctcagtttcctggcctgtagagtgggaataattataatactatcttggttgtcaggtttaaaagaccaaatgaatgtcactgcctggcagactacttaaaacaataagaggtgctaaagttactttaaaccaaccctctccaccctctccttccccacctcagtttcataccccagagaccaaattcctgatggattcctaaatgatatctctgccaaatactggtgccaggtgctgggcaaggcacagttttgcaaccagctgtcaggcagcaacaacttcacagtcgtacttttattcagtgtgtggtaaaacttctgggacaagagtcaaagtcgtagggctttagtgggaaactgccattgcctgaccagaggtacaaagcactggcccttccctcatccctggcctgacctaagagggctggggtaccacaggactgttgaccccactgagagctcagggtgccagaaacactgcctgccacgtgattcctattggtccttccagagatgctaacaggcctggaggctggaggtctctctgcccacactttcacacatgacctggagcagggggtcccagctctgtagctgcatttctgacccagttggataccttgacatggtcccagcaagcaacagagcacccattgtacagccttccccagccccagagagggactgtccctgccaagaacagaggtgccacccagaatctgacagcaggggaggctccccaagcattctacccagaagaacaagcaccctccccagatggcctgtggcccaccctgccctcagttcctagcagatcctcaacttggcctgctttgatgtttgtgaatggcaggtaggaaaggtcaggcaagtggaagaaacctaaaaccatgctctctatggccatggaagcttcctaggtattattacatagctgtagtatattaatattactgtcattaattgatattattgtaatgttactataatatacatagccataatataaacacattataaagctgtaatagcttatctttattctactatgtggcaagtctgtactaagcattctcctactctaaacccccacaaaagtattagctatgaactatcattgaatttcgttttacaaacaaggacaatccaaagaggccaagttacctgcccaaggccacacagtccacaagagcccaagttcccactcaggcccattcttttagctctgtgccacccagctctgcaaatgtagccacccaaagccaatcaagccagtggtagagttctaaaaccaaatctagggctctgtcccaccaatctccacctggtgcctggaattcctctatttttgttttttgttctgttttgttttaaagtttcactcacccccctccttaaacccaaatatccctgggacaggatggaaatccataaacaggtgcctgagtaagacatcattcacagagccttttcctcctgacagctggccaagaacaggagagggggccctgggcaaatctgcaggattacatcagagcttattttcccccgtatgcccctaattttctacatgctcttccattcctgatggattcctaaatgatatctctgctaaatcctggcgccaggtgctcttccattctcccagtgatccagaactttcttgctgtcatgaagaaaactgatttaataagtaaactcatacttaggaagcttcccatccaccctggccccccatggtatctcacatcatcatcagaccaaaagaacccataccctcatcagtgatgactcagcaaaatcaacttcacatgattaggaagctcacataactagcaaggcgaggaggttctctctgccggccacctcctcccgccagcactccaccacagcctgcctactgtctgcccgtttggactagcagctggtgctgtgggatctttaactccactgaaattatgtcacaaaactggggcatcgtagggaacctaggaaaacagacaagaaataaagactccatatgtgcctggcccacacccgagtttggctcactatgcacacattagatcctgccattcacaatttaaaatatatatatatttttttgtgtatatatacatatatacacacctagaaaaaaagtatgtattttgtatatatacaaaatttattacatataaataaaatttatacaaaatttattacgtataaataaaatttatacaaaatttattacgtataaataaaatttatacaaaatttattacgtataaataaaatttatacaaaatttattacgtataaataaaatttatacaaaattttgtatatatttgtatatgtgaaatattttttaaatgttaatatatctataatatttatatatttgaatatataagtttatatatatttatatggctcactataaaagaaattgaaacctccttaagcatgaacaactcccacgggtgttttcttcatgagacgcacaggaagatggcaatgggtactgaaaggaatgaattgcttgtgggggaactttatttactagaacaaccccaggtaggaggagaggttttaagaaatgagagtaggggttctggaggaggtcaccttgcctcctagtacagaggtaagactccactgaggctggagaataatgctcatgaggagattggcaggccccagatccctgcagctactgggacagaggcttcagttccttgccatgggggcctctccctaaagctgctcacaacatggcagttggcttctcccacagcaagagagagaatccccagagagagcaagaggctccacaatggaaggcctagtctttcacaatctaatcccagcagtgacatacccatcacttctgctctgttattggtcagaccaaccctggtgcaatgtgggagaggaccacacagggtacaaatcctgggaggcaggggcccttgggccatttgaagggtggttactccagcaggtacagagtatggtgagcccacatgaggaggtgcccagattgcagccatagatccttgggcaggtcatccaacctcatttacgctgtttgcacttacagaaaatgagaataatagactcgtcagggcttggcaatgcctatgaaagtgcattgaacaaagggaagtatgctgcagatataagacaacatggttttgattgtaattactagtaccagtagcacatagttgtgaggactcctattctgttgagctaacagccctgacccacctgtatcaatcagtgccaatgtatatttcacattgccaatacccagcgtcagataatgaagttttgcaaaatacagcttgggtgggatctctttaatgacctgttaaaactgatttatagttagctttttacctgaatgcacataaggaatctggagttcttgctagttcttgcttggaaatgatctgtttcttcatctgaatgtcctcccttcatctgatttccttagcaaccctggtttttctgttttgattgacaatacaaaattaaatattagtccagtccctcactgggctatcacaggttccaaaacacccagccctcatgtcattgatccaacatcaaccgttccaccggtccttccctcccctgcccccttaccctgccctaagggctctcaagacagacctgtttaactctgggcatttagaacacagaagactttctcaccagggaatctctctttctaaaatccccacagagctggtcaagcaagaagagcactggactacgcatcaagacctggggtgctgatcccgactctcatccttgactgcagtgtgggctgggcaggtcacttcactgctctgagcgatttagttctggttttgaaagactgggtgattctacccattattcactgagcacctctgggccttctattatggcctcctccctttcctgccctgcagctccctgccaaaaacaaagcctatgcaaccttcgtagatactcttcatagaatctgtttctctccctgtgggctctttccttctgagcactggcaggacttaaccatatgctctaggcattgggtgtgaaaccagcaaggcttggtgctggaaatttagcctcatcttcccaaccaccatagctaactcttgtcaagtacttgctctgtgtgccaggcactgttctacctcattaaaacttcacagcaacgctgaagtggctattatcatcttcattttatagatgaggaaattgaggctcagagagagtaagtaacttattcaaagtcacccagcccacatgaggcagagccaggattcatgcccaagcagtccagaacccaagcacgctgtcttgggactggattttctttccccatgaagaatgtaaatttcttaatcacaggccctgtttattttacacacagtcagcatctacaagtctttgttaactgattacaggtttctgtaattctaggcatgttaagcgtagggtcaagcataaactctgtccctacctttccagaacatatgctctgatatattatagcagacaaatgcaaaatttccaagactcgtgggatttagaagggatttttctcaactcttcttttccagataagaaaagggaagcttgaggagattaagttacttctgaccacatagctccctgctgcagaaccaggacagaaacccaagccttctcatccctatcacgagttggatctagggcagtggttctcagtggttctcaactgggatggtttcgctccctcaccccatgggacatttggcaatatctgggggcatctttgcttggcacagctggagaagaggatgttactggtatctagtgagaagccaggggtgctgttaaacatcctacaatgcaccagacagccttcacagtagaatcatgtggcccaaaatgtcaatagttctgaagttgagagatccattctgtgggatttaccacttcctctaaggctctttgtttctgtatctttacaagcagtcatctacagttggacaccttgtcttattcatccttggctccagtgctcagagaagtgcctggcacgtaataaaagctcattgaacaagggagggaaggagagagggagaaaaggaagggaaagaatgacagaaaaagatatcaggctttcctggcaggcaaagaaagtccaacactataaatcaagacctctgttcataaaacagaagatgtctgactgtttgaaatcaattcttgtatatcattccagagagaagactaactccgaataccttaattatgcgttagcacaatttagacgaatactcacaaaattaagcagagaacccaagccctgctacagaagtcttccccctcaaaaaaaaaaaaaaaaattaagatggagtctccctctgtcacccaggctggagtgcagtggttcaatctctccagggattacaggtctgcgccactgcacccggctcatttttgtatttttagtagagatagggtttcactatgttggccaggctggtctccaacccctgacctcaggttatccacccgcctcagcctcccaaagtgctgggattacaggcataagccaccgcgcccggcccttttaaagtaaatcttgattgatggacatgatgaagtccgtgaggtacaaatgcactgccttttacaaaagtaactggttccttggaggtgtccattgggaagatggcagtagttagagctgccactgaggaagacataataggcagctctcattccaaggcagctgagccacgaagaaggctggcctgagtggtcagtgtgcagtaaatccaaatctttaaatgcagttcatgcttcacccccgaccgaatgacaaatggctcccccgtcagagcaagctggcaggtgatgcaccactcctcccctcccctgccacccgtggtgggacaatctgtcagcagccactaattctcaggaatggatggggaagtgggggaagtcaggctgtgtttattgggcaaggagaaggggtctctgagcccaaagaccttcttgggaaatgcagctcctgccctccacagcaaataatgctcaatgaccagtcagggagagcttgtctgggctctcttcaagctatggggagcatctagaaaatgccttgaaaccgtcagatggaaacatcctctcatcttcctccctcctggaagcaatgtgctccgccgctcttccaaacccttcctgtctctactggtacacctctccaaagccctctggatacctcccatgatatgcctttcccaggctccaggtcagcacaatgctctccccactctattccccaaatgcatacctgtctccatcgcagacaccttcacatgcctggctggcaggagctcagccctgcccatctgcctgtctttgttgtgcgaccaccttcagggtcctcccttgggctcagcagtgagctgacttgccatggagccaatggagcttaggtgtcaagatccttcacttgcacaagcccctttcaacctcctctcccccaacacacaattatgtattcatgatgtattttttttcttaaagaggacctccaaattatatctaagattcaggctctgtagaacctggctctttccggccaaaatatgatctttacagagctcaacatgccttgcacacactaggtgtttaataagagctgaaggatcaaagagaatggatcctgaccatgattacaattaattaagaaagtgattaacaggaaagggaagattggtgtttctctcaggcgagctttcctaattcttcctctaccatcaaactgaaaaaagccaatagtctcccctatctcaaatccaggatcctcggaagggtttgttaagtcacagccagctggaccccacctcgcaaggatgtggttcaataggtctagagcgggacttacaatctgcatttctaacaaattcctggtgatgttgccgctgctggtccgaggccctcactctgagaatcactttgagcccctgcgttatcaataggggccatattgcccccaagagggtgaaattgtatcttgaggtgggtgatgaaaaaagtttttattataaagcagatatatatatatacacacacacatatatatacagtacacaaacagggtgcagtatatccctggtgctaaaacttcacgggtagacaattagggggtaaaaatgtctagaaagactctttaggggggcaatcatgataaaacattgagaaacactgacctggcccaagtgcctacttggtgcttactccccagcagctgagccagaagccctcacctgcagagacctgcagcccacaccatctgatactgtggaagaagcattagactgggagtcacaaaacccaggttcaaatcctagctctgcagtaacttcaggccttcccagccacacccacttcagcttctgtactgaaagagacaaagttacaaccaaaactgccagggaaagaatgctgtgcgtgcttggaagagatacatgagggggttgtttatacaaataggagtgagcaatactccacagggcattgttgagtagcaaaagcaacacattaaaccatctcccaaggaggctgggcctgtcaacggccttgcttcgctgagcatggaaaagcacgtgtcaggcagagcaggattactcggcacaagagaggccaggctggggtgggaagaatggagagacagtgcctgccagtgacaggctggggcagggcgagaacaccagggaacatgcacatcgatcctgatttgagcaagtgttctcagaggcagctctataaattccttcaccacccctaagaattttgtactttgtcgcctgcctctaaacaatgcctttggggacagcagattctttgggggtagagccaaaagaggctgagtctactttaatgttatgctggctcaaaaacagactcacacatagtgggaaccaccagactcctgggttacaagtcttgtgtctctgttaactatgtgtccaatctcccaggccctaaactaggattctttaggaatgagtatgtaacaggaaagggaagatctgtgtttccctgaagcagctttcataattcttctaccatcaacctgaaaagaaccaatagtctcctctatctctaaaccagattcccatttcttccttagaaactatgtccagtcagccaccgaaccctataagttgatcccagtggatcaactcttccgctccggtttctctgagcacctaggaactggtccctgtgtctctcctggggggatccatttcttatccaggccccagctctgcctgtccagcccaggagccacccgaaatgctaaatctgtcttcttgctaaacaagttggattgtcataactgtatagccctggtctaagtgaggttggataacagaagtggtctaggaaaactgtgtcctggggaggaggtaaagtgataaataggatgtggtcacggtgtcctaggcagtagcccctcacaatgccagtggatgtcaggatcacgaatcagggaggtctgtttcggtagaagacatgagttggggaggtctgatttggtacaagagatccagaacttgtgtccagctggagtaaggagagtaaggtatagcagagacaggacaaagtaaattggacctcagagacagggcacggggcagagtctgattccaactgaaacgtaaggatccccacatccctcaacagtccctcagatggtgaggtttaggacacagtaaggaggggtggaaagagcatgggtttgggtatgtcttccctcatgaaaaccgtggagccagaagcagtagtgttacagtgatgtcatcccctgaggctcagtttcctcgtaggtaaggtgggaatggaaatgactgctgcataggctgtgagatgattttgtgagatgtaagcgcccaacagatggtggtgatgacgatgttggaatcaggtgactctagcctcttcactgtcacgcttgctctcgtctgaacgtgcttccatgtctccatgttgctcttgtgtccacaagcaaaagcaataatccattgccatttaaccagcacaaaactgaatgaggctgtcaccttccttttctgaaacacatgcttcagacagcgcagtctacagtcacttgagggctgggagaagatgctgtacaatatttactcctgttgaaaattataatcaataagtccctgactgttttcatatgtgctgctactaagctaccactccctgaccttgagcagatagatttgggatccacgtgtagccctgttaaattttgtcttttcagatttagtctctccctctctcttctccccccaccacccccctctccccccaccaccccccttctcatcctttttgacacttttagatttgatatctcttctagcttcttgacatctgaaatgtggtgaactcatcctctatattatccaaatcaatgacaaaactcttgactaagacaaagccacgtcaaaacacaggaacccctacccccttggaagacacaccaggccattcattgatacttttggggtgcattgctcccattagtgacacaaccttaccattctcgcagccaatccatatttctcccatctttcctcatgggcatcactgagaaaccttagcaaatctgcttcccagacccttctgttctcctttcactccctcatttgatcagcctctgggtcctgctgactccaccttaaaatgcctcctgtgcaccctttcctctgcgtctctacaaccgcacgcctcacagggatttgtcatctcctcccagaacattgcagtgatttcctaacaggccccctcgcctatatacctagactctttctgggagggatttaaggtggctcagcctatgtatattcacatgtctccacctactcccaatgtgatcattacctagaggcctctcatactttttttttttttttttaatgcctgaagtttttttgtttattcctttttgttttccagctctacccaccagtatttttttgttgatgacagatcttctcctaaaatgacaaagaatttagaaacattttccatgtatttttaacatctgcgtcagtgaacaaagaaatcatagttgatgatggggtggggagggagaaaagggaaacttaattagctttagtttttataagaacttccatcagcgctgaatgaactggaatatgacatctgattgggcctaatcacaaggaagagtacattaacttgccgtgaatatatttaccagtttttacaataccatttttaaatctctacagaagccaattttcttacaagaaaattaccctaccaagattttttttagaacaagcaaacccaaatttctttcttaccctaacccagttcatccagctctgacactgtggcctgaggacagacactgatgctatcttggagcctctgcctctccacccttcaggaatgttctgcaccctgaaggaggtaggaaaacttgaggatgcaaagaaaaaccagacatctgttatgtctcttgatgttagggagtgggagtgaaattccagctcacccactctcctggggaggctagaatttaatgggatgcttgagatgcgtgggccgagggtgaggctgcagccaaagatgcttgaggctggacctgaaggtgatttagtgaccagagggcaatgccagggagagagagccttgcacggagttagtgacactacagttcccagagtggggcaggaagaagcaggcacaccatgacatcttctcattttctttccttctctgccgttccccagatgccccctcaccaattcatcagccctttctttccttccacacaagctgctcagcactttggctctgctctggaaacatttccattcctgtgctgctacagaacccaccctccccaccagattggatgcacctggacagcaggattgtgacttagctgccatcccgagttttacctggcacacgcaggtgctaggtagtattccttcaaaacaaggaaaaggtggtgtcgcatccctacttagctcctaccgttgggttggtttgcaaattttgcattgaattccatgtaattttctctttttttttttttgtttgtttttatgaaaacgtatcatttagcctgccttaactcttgctttgaggtcacaaatgtcaaagtggttaactccaagtcacctccaagaacattattcctattgggttaaggctccacccttttgagctctaattatctccttaatgtgtgagaactgggggaatatgttcccaaattagaaataagttctctaaaactaaggcttccaatgctgaagagaatgtacaaaatgagaaaggcatactcagtccttcacttacacagtcaaagagcaatttttagggcactcccatgtgccagacagcccaacaggctttagggaggcacaaggatgaagaggacatggttatcagcacccccaagcaactctcagcctgtagagctttcctaatcattcaacaggtatttgaggtgctgaaacattgtttaaaaaaaaaaaaaaaagcccaggtgcaaacacttctatcagatgtgccatgcatcattgacaatagtatacacatattatttataatatgtataggtgctttataaatattaactcattttatctccataataactgagctatggacaataaatgattattatggacaatctcttaaccctcacaattcagacagtgttgctaatcccatgtcaaaaagggggaaactgaggtgcagaaaagttaactttcccagagtcacacagcctcttaagtggtagaaccaagattttatttgtcttcctggtgtaagagaacttggtgggttccttaaaccctgttcaggatctgtcactggatttgcctgttgtgcattcagaggctggagaaggagtggagggggcagagtccaaggtaggacactaagagtcagggagaggtgatttgtgaaaggctttaggggcagcgccagcctctggataacagggaccctggtggtaccaaggaatcattggcaggactctgggagatgaggacttggtctgcatggggcagcacctcgcatctgatactggcgcatatcagcaccctactggacacattcaaacaggcagcaccctgtgagtgtctcagcaccagcacatcaaccttccaattcaccaactcactccagggaatgtgaagggcagggttccactgggccgcaccacccagcccggggtggccccaaaggcagaggggatgtgagtgctgtctctgtgagcctaagtaggccctgcacctgccagagacagggcacagagcaagtgtctgagggcaagtcacctggacacagcagtggcagaaaccaaggagtaggcatgccaggcaggtctccagcagcctctgagtttccaggcacagggcccacaccagggtggactttttatatgtacatacacacccacacacccacacccccacccaaacacacacactgcaccagtcccgtttcctcagatggacatttttcagaagaaagccaatgacactagcaagtccatgacccatttcaagatatgacaggacatcattttccaagtggagaaaacaaaaattctcagaaagccctgtataccacagaaatccatacaccagtttcaagtctcatccaacagaccagccagagtgcagggccacagggcaccctggctggagtgaacaagctccctccaaaccccctcaagattctaggcccaccagcataaaagtcctcttgatggtggggtcattccaaccccatctggggttgtaacctctcagagcagaggaaaataaacaggctgttgtcaggcagcccagggcagaagtgatgacacctgctcattctgaatttatctttattatacattatggccacctgtggaccattcagaaaagatgctttttgagtctcaagcaagtgactctttaagccgaatcacaaagaaattcccattttggccagttctgtttctggacgtcaacacccccctctctgtatgaataaggggtcttccagatggccaggaacagcactcaacaggccaaaacaaagcagagggtggcttgagaactacactgctacattcagcctgaggcccagcatcgacctggccttctcccacttccaacaactcattttgtagctttttggttaagaacaaatttggatttcttctttttctcctcccagtaatctcaaatgtatcagaagaaaggaaatttctaccattattgtcagaaacaagacaagtaaaaggccatcctcaaatactagtgttctcttcaccagacagcagcacacgtggagagtagcagatctctaagcacgacccagtgtgtaactccaaatggccccattatcctatctcgaggagagctgcgcatgctgtaccctgttttacatggcctgccatgcttctggtagcaaagcagtaatctcctggttatgtagcactggggatgccaataacagccagataaagaatagaactcatgaggccagttattttcagtcaaaccaagctacaaaaaccacagtcatgcagaagctggaggaaccaccaaggcaaagagaatggaaattcccttatgctagacagcacaggtccccagttttctggggcatctgaaacttgatgtgacagcactgggagaggccaacagtccacgaataggccaggacctaaggggaaaggctaccactctacagccggccatcagggtgctgggctacagcagggctggcaggagcagacggaggccacatgccaactccagcctctgctacatatggtcccagcccccctactcaccagccatgcggcctcaggcaagtgggataatccccgtgtctcagtttcctcatctttaaaaagtgggataataaaagattcatgtactttatagggttgttgtgaggatcaaatgatttaatatacaaaaaaatgttgacaacagggctcaacacaaagtgtgctcaacagctgctgttggtattggtggtggtggtgaatggtctgccagcttggtagggatgtctatgaagtgggacaaacatttcatgagtatgaagatagactgttcagatgatactcatctaatgccattggcagctcctctctttattgtaccaagatctgtttccttcacgtttcacccaacgtcaaccagcccttcccttctttggactaagtctcccttcaacacatcctcaaatgacacagtttccagtcccctcactcttagtcaccctccttggaacaagctcaagttgctaaacaactatctctaaagccagttcccacccgagccaacattccaaggccaaaaaccatcacctctatcatgagttgagtatcctgctttcattaggggactgcactgacgtggctgcaggttgctggtcagccctagggagccagtaccatacagccagtacccgcgagctccctcctcacaagaggcggctgttgcctccttgtgccagagctcctgggctttggatagaggaattccactgcttgggtctcatccttactcaccaacttggaggaacttccatagagtggatggcagccgcccctccccactctgtctctgtggtgtgtgccccacctaggctgtgtttgggtgcctgtagtacctcttcctgttcttgtctcaaacacacacctgtggggctcctttcccagtaccgtgcttcacaacggggctctccttagcccaggaggggagaggctgtgcagaaaggagacttctatgacacccttggggcaagggtgttgttttgctccttccatagatctctgaagccacagctatgcaggagaacagaaatagaagccagccacacccagtcttctttggcatgggcacattgaacgggctcagttctcctgccacgactgaaagggcaccctcccacagaaggcacatgaccactgttccaagacagtccagatcaggagagaggcctttgaccagggcaaagcaggtgctgatgagaaggcacagcaaaggtggcccaaaccactctgcctattccctatccctcccctgttaactccactcacaattccaaacccagagccctagcttctgagatatccaggtaggcgacaagattttctttctctacatgatacagactgctgagaggctcccctaacaaagcaccagtattctccctgaagacacaggctcaaattatgctctattcaaagctggctgtgttcccaactcctaagccctgctggctccctctgccccccaccctgacgtgggggaagatgctacaggtgtgggggtgtgttagtccattctcacactgctaataaagacatatttgagaaatacccgagactaggaaatttatagaggaaacaagcttaactgactcacagttcagcatggcttgggaggcctcaggaaacttacaatcattgcagaggaggaagcaaacacatccttccgcaaatggtggcaacaaggagaaatgcccagcgaaggggtaggggaagctccttataaaaccatcagatctcgtgagaactcactgtcacaagaacaagctgggggaaaccactcccatgattcaatgatcttcacctggtccctcccaggacatgtggggattatgggaactacaattcaatatgagatttaggtggggacacaggcaaatcataggagtggggaaaccaggagaacagggaaataaaattggttttcacaatagctaaactttccctatcctcttgagagtatttctaagcaaaagagaaatatacttttctttttgctttctctaaaaaaagaaggaaagaaggaaggaagaaaaaaatggagggagaggaggagggaaggaaggaaggaaggaagcaaagaagaaaggaaggtcaaactgacttgatttagctcattattaaacatgcaaataagaaaattcctgttcttggaatgtgatcagtcatctgcataaggtcttgcgacatatctacgattgaattatttctttgaatcaaggattcccccggccacacacaaatcagtacttgccttactttggaaaacgaaaagagctcactaaagtggtccctcttataataaggtgagagtggcctctaggacaaatttcagcttgtacaattctggaatacagaaagggtagctggctagcgacctttttccatactccaccaggcactgtgtagaggctaagggaaagcttcccctttgccctctgaaagctcactgaaaactcacagaagacagattagtagaagaaaaggcatacgaatttattttaacatgtctagcactggaggatccatagagaacaattacccattaacccttcttcataggggaaggggagatgggggaaatgtggcaatttgagggatggtaaatgatttttagggggagacgagtggacttggggaacttacaatggccatggacgaagtctgttgggacttcagagcagacaagggctggtaaatgattctctttgaaatactgaatgagcaccaaaacagaagacgatggtttgtgacaaaatactgtccaggtgtgttgacagacttcagtctttcttcctgtgatatgggttcagttgatgaaaactcagggaggagactagaggtaattgctttcttccttggtatttccagactttaggcagataagggatgaagagaacaacttcatccagagctttgggagagatgaagaattgagagatggggtagggccaagggtggggggtagggagtgaagatcagagagaccctgaggctcttcttatttcagcatgtcaaagcaccatattttggggtaacagttgctgagccccaacaactgccaccccatagtcaaattaagccgatcatccccaaagcaatattagtccagtccagttcctctcatgacaaatgacctgaacacaggattcagcgagtcttactcgctgtggtcattttggaggtagggagatcattaacccacatactttgataaaatacgaaacgcctttcctagctggttcttttcctctcctataggccgacccccctccatccctttattagtattgaactgaacaaattagttaatatgggaacatttggatgtatcaactttgctctgaaacaaaaatgtattcatccccaaactctatgctgggggctgagggagagtccttacgactgacatagtccccaacaaggttctcattagtcccagtttttttttggacaccttttcctccgcattcacatccttctgccctctccagcactgtgcaggaagaaccaaccaacctgaccttggtgaaagggctgtgggtttttcaacccttcattcctgctactttttttttttaattttttagtattcttctgtgcatcctttcttctctcctaggcttctcacccagcctctctgttgagtactacttgtaggcagagctccctcattcacaacatcccaatgtatttgtcctgtatcaaagcaaaccttatacagtagctcttttaagaactgctaaaggaatgaaggaacaaaactgagccaaataaagcaaaatgaagaggatatgtatgcaggcaattgtgggaacatatcaccagaatcctctcttgttattgtacccaaatccacacattctcctcagtgttgtctgggaaatacatcaatccactgtgaatggtttagatagaatagaatccatcaatgtcaccagcttgattcctcctcagatgatgagccactttctccactccacctccagggctggccccagtcttcctaaatgttcattaactcctattaggtattccgaaactctcaggttttctgagtcagcatagaatggttggaaaagcaattgctgtagaatcaagaagactgaaattggaatactggagcaactacctactagccacaggcaggttactctctctgagtctcaggttccccatttgcaaaatgaagcaaagctatctactccaaggattgctgtgaagattaaaagaccacgtgctaacctaataggtgttcagtaaataaatataattttctatttttccctgcaaaattcatttcccaaacacaatctttattcttttttttaacatataatagccatactatggcttaccaactccccagtaagccaggactctctgtatacgcagtgttttgccacatcatttacatggatgaaattgtttcctatactcccaaatttaccctgcttggtgaccaagaatcacttctgtccctattactagtgatatcatgtctgcatttagaaacagacaagtgttatacaaaattacataatatgataggaatgctgtcaatggatatctattattatatttaccagtcaaaattttagttgttataactggtatcaccaattaaaaaattttataagaaaggggtttttgtaccattggatctattgaatcattaaaaaaaaaaagagtttagaaaaagaagaagctatttatatactatttcattccataagactttatccagttccactgttaataaaatgtacatgcgcagaattgtattgttttctccattactaaacatgtatttgctgagtaccttctgaatgccaacatggtgctaggtactgggcatagggtggaagacacaatagacaaggacacagctccctggtatgggaaacagatactttatgtcttcagttgtaaggtccactgttgttcctatttaatatctttgaatgtggaatatatcttacaatgctgtgggctagggcatggttgtgacattgataaaaagggctcttaactgccatcgcttcagttgacttgcttgcctttttgagtggcagtgcatggcttgagcttaatttccattcagaaggtttttgaaaagactaactcattatttgcacataaaaaggcacagagacagaggtgaggcataaatttgatattcatcattagagtaaccatcatcctatgttttcttacaaagtaacaaaaagtgttttatgaaatgtaatcacaatatatgaagctgtgctttactggtgcacatgcaagaggattgcccatcaggctctaagcaaagcaactaagagtaggagaaactgccaaaccctaaagtaggggaaagaaatctccaaacaacaagaggcactgtgaccaattcacataagactatcactaagacatcaggttttaacttgtaacttgcttttttccttgttagtggtatgttaaacaatggtatatcatgctatttgtgtatactggattcaatgaaatactataataaacaagtaagcaaaacataagatcataacacgtcctgaaaggacctctcagcaagagtgacacattgccaagtaatgcacattgttccacatggagttggtgaggataacagcacttccttccagaaatactttagtatacaaagtgctgtcacgtgatgacgtctcatttcagcatctcaacaaccgcacctggaaggccttattattttgtatatttacggatgaagagactggatctcaaatagcttaagagacttccctaacataactcagctaatgcctggaagacccaaaactagaatccagggctcctgactctacatcctatgtttttcttctcggctacatgaaatggtacaaaatacgaattaaatgattatccatggtcttgtgtaattctggcatgtttccagctaagacttctcagctgttcattcaactcaaaggcatccttcagtcctcttccccattcttctctgtctcggggatctgcctggggagggagtgtggtgaaagcagagtaactgaatcctcactaaccccaaggccctaacgcaaggagatgcaggcaataaatcattccattgcatttaccattgcattgcatttaccattgcattgcatttacccatacgtggggctctccagggatggcatcagatgctgcaggaacatgaatgataaataccacatcccaagggttagggcccaggcaagttctcaggggagagctatacatatgactggtaaaacccactatgtccagaagacaagggcaccctgctggtttgcatgagcaaagctaaattggcgctgacaactggcaataggtattgagagtctttttaaaagttgatgtcacttaatctagtaatcccatttccaggaattcattcaaagagaattaatctgggatatagagaaaggtctatgaataagattgcatcacagtgtggtttacatttggaaaaaactgtggttgtaacttaagttacaacagtagagggttgttcaaatattataattctggtacaaccacacgttaggattctctcttgccaattattgatgtcgtgggggagtgttcaagacaaaacattgagttataaaatcaggattctaaattatatatatacacacacatacacacaattaaactcaaacatacacatggagacacacacagcattttaaaacctagaaggaaatcacaaccataatacaatagttatctctgattatgggaatacaggtaattttggtgattttctctatgttttcttttttttctttttcttttttttttttgagacggagtctcaatctgttgccccaggctggagtgcaatggcatgatctcagctcactgcaacctccttctcccaggttcaagcgattctcctgcctcagcctccccagtagctgagactacaggcatgagccaccaagcctgactaatttttgtatttttagtagagatggggttttaccatgtttgccaggctggtctcgaactcctgacgtcaggtcatccacctgcctcggcctaccaaagtgttgttgggattacaggcatgagccactgcacctggctgtttttctctaagttttctacaatgttttattttgctttttaagacagaaagtccacgcaaatcagtcaggaatagatgtgaaaaatggggatggggcctttgaaccaggagcaatatgagaaggtgagaaggatggcctgggccggctgggtctgcgagactacctggaaaagccacgggaatcacagcccacagggtgacatctccagatccagcagtcttttaggaaggcagatggagtctcagacctgtcccacccatcttaaagtggcctggtcacagggtcagagcttcagcagacagaggcgccctgggataagttgccaaatatgggttgagcaacagtggcactggaacgaggtaggaggcggggaaaggggatggaaggtaaaatcctgcccctgtcctcaaggtacttacagtcaatgtgaggaaggctgacagccataaaatgatgacctatgacaacgagggtcgttccacacacatcctccagaccccggcacaagacctccgcctttcatctccattatattgggcaaaacacaaagcatcaacgaaaaaaaaaaaaaagtaaaagtacatagaaaaggagacacagaggaatacagaaaagcaaaaacctccttcaaactggtggatgtttcatcccaaaggacgctcgacctttctcaggcatcctgccactgaagaagtcaattactcacggcccacagtccctaggttccgctcagcacagtctggaatcaagggttcatttaatggaagcttaacagattcagatttctctaatattccacatccccttctgccaagaagctgctttgaaagatgacactcccttgatgtgaatttgcagcctgccccattaacaaggcttcagtcagaaaacatttttaaatcaaccctgttccagacacttgctcccaaggacaaactagaagaaactctgcaccagctcaaaccttccaagcactttgatgatcagaaatgaagggaaatattagaacaaatcttattaatccaaatattctcaacaatcttcaaagcaaaccgttgctctgggctgcagaaagcagagctcatgcagagccggccctttctgtcctaactcctgttagccaagccagccccctaaaaacagacctcagaacagcagagggaaatattcagaatcaaaggtgatgggaggaaaaaggaagaggaaacaggtaaatgaaagaaggaggatgaggattggaaagctgactacttaaacttttagcacgtccctcaaataaaaggataaggtgttgtatgccacactgcctcaaagcaactccctcagttcacagttgtgcagctgagaattctggagaaaccccacggcagctggcatctatgtcctggctccactcacaaggggctgagaaccccaggactcctcaccatgcacatttctcttgggtgtggtgggaggagcatttccgtttccaaacctcggggccatttgctcttagatacacaaacatctccccagcctttgccaagttcacccagccagaaggccataatgcagaattagtagcatttcaatcatttccgtgacgagctggtgacaggctggaatatgatgagtgaagcaatcagatgttgttacaggaaagaggcatgagaagagaatgacaacagagtggtacagtaataagcatcatcgtaaaagcccccactggcatcaagtttatcatcgccagcataactaaaggagccagaacacctgtttgtaatggggattccagctcctccactttctagctgtgtggctgagcacgcttctcgccctttctaagtcttggctgagcatctgtggaaggtggatagcaatcgctacttcataagattgtttagtgattaaagtcaaacaatgtctataaggcatttgcaaagcattagcagtaccagtagtaccagtagtagtagaagtagtagtagtatttggtattataaggagtattttatattatttcaaataggattatgcactatttcattatacccttatctcatatagaagacatttttaatgctatttgattgaaagtagaccagagactcagaaaggttaaacctctttcctagtaagtataaaaagttaatgcaggccaggtgcagtggctcacgtctggaatcccaacactttgggaggccaaggcgggaggatcacctgaggtcaggagtttgagaccagcctggcaaacatggcaaaaccccatctccagtaaaaatacaaaaattagccaggcatggtggtgcacacctgtaatcccagctacttcggaggctgaggcaggagaacagcctgaacccaggaggcagaggttgtggtgagccaagatggtgccacagcactccagcctggacgacagagtgacactccatctcaaaaaaagttaatgcagaaataaggctgcatatcggtttcctattgcttctgttaacaaattactacaaatttaattagttaaattgacactatcttacagttctggagtcaaaagtcctaaaattaaggtgtcaacagaactgtgtttcttctggagactctgaggacagaatccgtttcctcacctttttcagcttctagaggccacctacgttccttggcttgtggctccttcctatatctttaaagccagcagcatagcatcttccagtttctctctctgattctaagcctcttgcctctttcttataagaacccttgtgattacattgggcccattcaaagaaacaaggataatctccccctctcaagatccttaccttaatcacatctgcaaagtcccttttccatataaggtaacatattctaggttctggggattcagatgtggacaaatttggaggtggtcattattcagcctaccataaactggatcctgttctgcagaacatagctatttggagcaagaatgggagttttgtttgctttgctttaaactctttcatttctttactcagcaaataccatagagcagctactatgtgcagggcacattcataactcataacctcctcctctctggatataaccacatcagaatcgagcttcattagtgtgatataaaagaggtctttgcatatacacacacatatatatatatatatatatctcaaaatatcaccaaaacccatactttaaaaaacaaaaagggcctttggagatcatcagccaatccccttattctataaaggggcagaggagtaatacaactatcccagtgtcaaatagggagtttgtgagacataatgagggcctgaattagatctggaggaaaaaaagtaaatggaggtatttttgagaaaaatcagtgagctgaacggcaatagggtaggagaaatgtaggggtcaagggtgcctaaagttcatggcttggggatgtcactcacagagctggggacagatagaggaaagacacttttgagggaaaatatgtattcagtgtgttctagaactcggatctcttcttagtgtcactgtgaaattttccattacttcatctcacttcctgttctctcataagcgcgaacacggggctgtaatcatcgtctgaccaccacatgttagtgcactgtgtttcttgctgacacctggctaggctttgaggatgagtttccatcccaacatctaccaacatacaaggagcccaaggcaatgggattttctgcgcatctttgcttgaaccttaaaggtgctgccccattgtcttctggtaggcctcgtttctgggaagacgtctattgaattcttatctttgtcactgagtatgcagtttgactttttacctctagcttctttttttttttttttttttttttttggtcactggttttcagcaatttgattattctgtggcttggtgtagtttccctcatattttttgtgttttgagatcattgatcatcttggatctatgtatttagtgctttcatcaaacgtagatatttttccaccattattatttctttaagcatttcttctgtcccctttcttgtctcctttggaattttcaattacatatatatgaggcctcttaaaattgcccgacagctccctgatgttctgttaatttattttcagcctttattctctgtgtttcaaatggatggtttctgttgctgtgaattcaagtccactaatcttttcttctgcaatgtgtaatctgttattcatcccacctaatgtatttcatatctcagacattgatttttgaatctctagaagttcagtttagacttcttatatctcccatatttcttcttagcatgcttacattttcttctaccatcttgaataaatgaaatatagttataagaactgttttaatgttcttgtggactaattttaccatctataacaattctaggttggttttgattgattgatttttctcttcattatgggttgtattttcctgcttctttgcatgcctggtaatttttgactagataccggacattgtgaattttacctaattggtgctgggtattcctacattattttaaatgttcttcagctttgtcctggggtgcagttaaattacttggaaatagtttgacccttttgagacttgctttaaaactttgttagatgggaccagaacagcctttagtctagggcaaatttgccccttgaatccaaacactgtttatgttgttaatgatgacaaaatatccatgtcccacactattccttactaagtctcccttcaacaattgtgtttgagtagaactgttaactgcctcacctcactctcctgtgtattcacaaggcaaagatccccaaggttttgaccaatacccaatgttgttttggtgccagcttgcacagcaccagggctcttgcctctcagagagaattgtgccccctctctccactatgtggcacagcacattgagctcaacaacgttctatcagactgatgttgccataacactgcatatcttcacccaggaggggcgacagccctaagtctcttatgtggcaaaataaaatccaactttcagtgctaaaaaggccctgcttttctggagcgaaccccttcagaggtctcacaagcatttaaaagagaatttctagaccacaagtttcagtgtggagagctgcaggcttagagaattgcagggatgtttgaagccctgcccacagttccaggaaacaccctttgtatatgagctcctttcccacacaaatgtgagaaggaggctatgaccctggaaattgaacacagggcaacagaaaccctgccgaaactattcttgcattgacattagacacaaaaacacccatataagcttgatttgctgtcacagtcaaataaaacaataacatgtaagcatttcaaagagattccactgacattctcacttgtgattctgacaacaagcgtgtaagtaagaaaggcaggcgtcacagctcctaagtgacagacgaggaaatcagccttgcggaggttaaatgatctcaacaaggtcagacagctagttggtgcaaagtggggccctgaacccagatcttctgactcttggtcaagagctctattcattccgctgtgtctgctctcttactatatcagaaaagggaacagccaccaacttcctgtgttgaccctagaccaggacttcttgatcacagcacacttaacatttggggctggataattctttgttgtgagaggctgtcctgtgcattgaagggtgttgagcagtatccctggcctctacccactaatgccagtagaactccccttgccccctgttttgacaaccaaaactatctccaggtgttgccatgtttccaaatgccccaaggtgggggaaggtaacagcagagcaaacttacccctcagttgaaaaccactgccctaaatgatgcctgagaatgctgtccacgtgtttgagaaagcagtggtgattaagttagttctgtgaaaggagaaacaaaatcaaaagaggaacttgtgctgataccagtgagtctgggaccacgaaacttggccgtgagagacagaattgtgcagcaccagggagttatctaaaaagttctcttctgggccaccctctgaacttaaccttctgtgttctcgctccactccttcaggctcagtcagctctgcaaaacccaatccactttccaaggaaacatttctggaaggcaacacggaattagttaatgtgatgagaggcagaacagagcagttaagatgcaggctctgcacataccacaacgtggatgaatctcacttaatgttgggtgagaagagccaggcccaaaagagcatctattgtgtgaatctactaactcgaagttcaagagcaggcaaaactaatctccggtgatggaagtctgaacagttacatctgctggtggtgttgcctggagacacgtttcagagaatagaaatgttctctgccttgatggtggtggtggttacggtggttgtaaacataatagacactcacaacactcacagagctatacacttacgattcgtcacctaaaaaaaaaaagaaagaaaaagaaaagaaaagaatgttgactctagaaccaaattgtctaagtttcatccctgacgatgactctggttaccttagagcattttgtgaaaagttaataaactacacagggtatacaggaagtgcacaataagtatttgcatcattataattaacagaactacacttcagccaagcgcagtggctcacgcctgtaatcccagaacttcaggaggccgaggcaggcagatcacgaggtcaggagatcgagaccatcctggctaatatggtgaaaccctttctctactaaaaatacaaaaaaaaattagccgagcgtggtggcaggtgcctgtagtcccagctactcgggaggccaaagcaggagaatggcgtgaacccgggaggcggagcttgcagtgagccgagattgtgccactgcactccagcctgggtgacacagagagactctgtctcaaaaaaaaaaaaaagaaaaaaaagaactacacttctagccagtcaggcaaagccaaggattaagagagttactctgaatttctctgctgccccctctgcctcacctcccatgcattgaacacagagtgtatatgtaagtcctcccaccccaaccatcaagacaggcacatctgccatttttctctgtcttcttcagttccttccaggttctctctgccttgcttaggagctgcccctgtttgcagagctcttcatcaatagactgaatatttacagaatagcgcagttttgattaaacagttcatttcactggagactggggtggtgtattgtgtctcctccggagttattcttggcatcagcagcccttgctcttagcatccgtttctttacattttaaggtggagcataagaagaactgcagtgaaacagatcagatggggggcaggtgggatttctctttacctttcccccgctatttcttggtggatttatcccgttatcttcgctgctatttgctccttgctatcttttattcttctcttttgaaactggccgagtcagaaggaaataaacaaatgcaagggtgcgtagaaagagcattagatggtaggtccagaaacttggactctacatccagcatctagtgatgtgattgattgatatctgttttagcttttcggcacctcaggttttgggctgttttttttttgttttttttttaatctgttaattgaagtgattggtctagatttgtggtctttgaatgtgtgtcagccatggaaaccttcctacaaacagaatctcaagcagcaaatgttaaaacctaaaagcaattttgtttgggttgaaacaaggtgggcgggcagtcaggaacccacccttttaacttctgtgtcctctgccatctcctgtagcagcccccaaagtagttccacaagatccttggccttgggatgctagagcacaacgtctaaaccattcgatgtgcctatttagaaactcatttcctgctcttaaagcgctcagccctctcccctacacataaagccttgcctccatgaaagcagggaggactagccttcgaaaccccagcacacagaggtgagtgtgagcacagctcaacagtatacaagcccgctggaaagctgccccagacagaatttcctcagttgcatcctatggactatcagtcccagaaacgcttggccaaaaaaggaaatacggagggcgggggcagcttgcttcaaagtaattgaggttgagagccgctgtataccatctgccccttctagagattaaattcacagtagcttcttctctcgggccagcaggaagcctgattaatgccgcttaatacgacacttcccatatttatttggccacaaaactcctttttccccataactcctattaatatcacaggaaacagtttgagatcagaaaagccttcacaaaggcaatttttaaagactgagttataaggctcaaaataaatgaggaattctagaaaaggaagggaggagggagaaggacactggtttggggtctggagggctttaagatgagggcgaaacaaggaccacccatgattccaaagaatatccggaacttgcttgttgtctgttggtgcctttctttcagctcattcctagagggcggacagaggcaggagttgctgtgttgctggccagtggctcctgaccataggaaggggactgtgggtgtgagaagtaaagcagagcaggcccctgcctctgttctccccttccccctggtattggctcaccgtgacaaatctttccctaggaagggcagaaaatgaattatcctgttaaatcaattcagcagataacccacctctgtgcaggccttgtgctgggaccacacagatgcataagacacgtacagcttagagcctagtcatgaaatgggaaataaacagccccatcctcaaggtctgaacaggggattggggagcacagagaagggccccaccccgaccagctgtcaagagaactatccatacattctaattcctccaaggttgagggctaaggtgtaggaagaggagagaggttccaagcagaaggaattgcagaagcaaaggcacagaggcatccgggacctgcgagcagctatggctggagccgcgtgtcgcaggatggagagcgtgtgaacagaggggactggaagcggatcctcccaggaagcagttgcaggcggtcatgctacagtgagttgctgatagtaggaggggaagaaagaggaagggggagtggaagatgactcagtggggggtgagccagtgcctggaagagaggaatgtgtgagcagaatgaggaagttagcaaaagctggttgaaggtaggaggtggaaagacaagttcagccctcaacaagttaagcaggttatataagaagtccaggcatggatgacgatgatcataatcatgatggggaggatgacaaagatgactaggaacgctctgtcatctctctctctctctctctctctctttccttcctggttgtgtgagtttgcagggagacgggaggttgtgaagaacaataaccagaagagagtttgcgtcttcatcagaaagcacctggggcaggtagagttggtgctagcttctccatccatccctgacaccccagccccattcatcattcagcagaagacaggctgggctggcccaacttggtcctgtacccccaccaccccagaaccccactggtccatagtgaggatcccttctctttgactctcctccttctctgtgactcgcctaagcctgatcctacaccttctctcccagccactgcctcagcttcctccacccccagcttgggacgagccacctccctggctgggtagcatgggacaagccatctccctgcagaggcagagaaccagtgacactacacgcaaaccaaggccatgctgcaaacagaacaccaaggcaccctcgtcttaggatgcattatattcggggtagatgttgaattctagtaacacattccctggggtgttttcaaatccagacacactggagccttgttccagcctcactgtgggcgaaggccccgggctcacctccagcctcaggctggctctggctgctgcccctgttccatcctggcaagcggaactgaacaggcccgactccacagcccccggtgctgccagctgcaagctctgctgactccaacctcaggcccctgcgtgcctgcttctaaatccacacgttcctgctccttcctgcccctgctgccctctcagaatgccacttctttctgccaagacctcaggcattgctatggcccagccccatctttattgatgtcggggctctgtccccttgcagagcccatcccattgccacccacatctgggacagcaaaagacttctcagtttcctccagagttcagtttcttccaaaaagctcggcacatcttagctgccagtgtgaactgtgagctcccagagatgcccgtggcactttccaacattcgtcacttttgacacatctaaaatatcctagctcccagagctgcgtgagcaccagcccagatggaccggctgctggggaagcatcagtctctccagggcctctgtgaagccctggggcagtagggggatgtctgacaaagcttcctaactgaggagccccacttcctctcgctctgggcacccctgccaaatgagcgggcacctctatgttccagcagccctcgctgcccagcttctgagctgcaagccagcttccttccacagaattctctgcaccttggcttccggtggctccaagcactttgccatctgccgagtcagttctgccagagatgaagcagctgatgtggggagttctctgtgggggccacctttcctctctcaggtcacctttactctcagaaggaaactgaccccctgacctattaaatagctgaaatcaagggccactaggacccaagtcggtgaaggtgcaactgaaggcaagaaggtcagcatctagctagacagacacgtaagccaagaagacagacgaggtgagtggaccctttgactccacagtcagacagcaagagagctactcccagagttctctggggcatgaaggggctgcaggggtaggtgaacagcatccagagaaatagttatcatggaaagtgcaagcaggaaacaagctgggattttttttttatttaaaattatttttcaggccaggcacagaggctcacgcctataatcccaacactttgggagcccgaggcaggaggatcacttgaggccaggagtttgagaccagcctgagcaacacagtgaggccctgtgtctatgataaataaataaataaataaattagctagtggagcagggctggtggcatgtgcctgcgatcccagctacttgagaagctgaggagggaggatcgcttgagcccaagaggtcaaggctgcagtgagctacgatcactacactccagcccaggcaacagagccagaacttgtctctaaaaatacaaagaaataaaagaaaagaaattcttttaaggaagtaaatactaacatggctcagaaattaaaagaatgtaaaatagaatagatttgcaaaatctcacccccatcccacgatgtccatatccattccctcctccccctttcccaccatgggtaaccacttatattcatctcatctgtatcgttccaacataaccttacgcagctgaaagccaacatggagttatttacttatgcccccacccttccacaaaagtatcacactttagatactgttctgcaccttactttggcaactcagcagtacattctagaaatcttttcaaatcagtacagagtgtttctttagtcaagctgcaaagtattccattgagggatatgtccccgttcagttatctagacactagatggacacttgggatgtttccagtattttacaattacagacaatcctgcaatgaatgaccttgcattaatttcatttcgtaagtgtgcagatatagccacagggtagagtcccatgcaggaaatgctgagtaaagagaaaatgcatttgtaactttcatagctgttgccaaatccctctccattggagttgcaccattttgcattcctgccagtgtctccatcaaggtgtgtggtcacactttttaatgtttaccaatctgatggttgagacttgtgtctcagtatatatttactttgtatttttcatcttaggagtgaggttgaggatcttttcatatgtcaaagggccattcatattcctttttctgtgaaacggctcctcatgtccttggttggcccattttttgttctgtgggttgctgtccttggtctccttgatttctaggcattctatagatatcggggatattcgccctttgtgttatgagttgcaattattttttctcactttattgttcattttttgcctctgcttatggtctttttttaatttagttggtcatacagaagttttttgtttgctttaatgtagtctaatttaacaatcttttacagcttctgaattttgaatcacagagggagattcttattcaaaaggttatcaaggaattagcccatattttttttctggtcattttatgattcctgttatttcatttaaaaattggatccatttggagtttttcctgatgtatgatgagaggtatggaccaaacttataacttttcatctttttccagatctcccaatactattttttaaagtccatctttaccccagcactttgagaaattagatttccactgcccagaacaacctggccaatttgagaagtcacttctatcaaatactgaatgcaaacatatgcatttgagtgttctgggtttctattctgcttctttatccaagctcctgtatacacagcttgaattactgaatctttacggtacgtgttagtatgaaataggacaaatcttcccccatagtcctttttagttcagagctcccctggccatttactggcaagctgagctctgtcactggggctctccttgctcactgtcatgttttattctcttctctcctacaaggaggccaatatatgcctgcaggcatccaaccagggaaagaggcaagcacagcccactccccaaacaaggcagaggcaggggtgcaaggccttctgggagagtcccagtaggccatgtctatgtacccctttgaggccggtgttctggggaaaggccaaataggagacctatgtcttcctggaggtttggactttgccctgaagattctcagcactgatcttctggtccatccaatctgtttaaatgagggtgatcccagataacctcaacataaagagttatcctgtctgatattctgtctcctcatgatctgttactgatcaggtgcagaagaacccttcaaggaggaagatgttacaatagcaatatcaatgcctacttctactaccaccaatgccaccagggccacctttattcattgagaacctgctatataccaggcattgtgggaggtactttacacgtttatctcattcacttacaaccccatgaggaaaccatctgtaaacgtgcccagaattgtggctcattagcggcagagccaagctttggatcccagcctggttccaaaactcatactcttcaccactgggcaacttcttctcagaatgatctgccaccctgggatgatgcaaattcccaggcctcccagacctactgaatcagctgctctgggaatggcacccaggagtctgcatctccagcacatgaatacatgagaaccactgctcacaaatgtaaaaagttaggtaatatatcagggaggctgtgaggatgagtgtttccttagcagtggtgtaagggcagaaccctggctggagtgttttgcagtcagactgatttcctcattagcagagagtgttaaccggtagcctagtttggctcctcgaggtgcctgattcagacagagcatcccacagctggagcaccctagctggtggaggtagggactcagccaagtaggctccagagctgccacctaagcccagtagatttctcttgcccagaacaacctggccaaatggaagatgggaggctgaaatcctcactccaaggaggggcctccttttcttggcacaaaggcaccgcgccaagctacacagctaaggcagtgtgctacctaaagcaggtgcacagaattcacagagaggtaccaaataggctggcaaagaccttggtggataagtacccacaaatatctggaaggaactatgttgggttgagacatgagaaggaagagatcttaggcaagtttggaaagggaaacaacacattgaatgccaacccctggactagatatttcatcaacttcgttcatcctcactaccacccacacaagacggtcttcctatttccaacttagaagaaggaagttgaggctcctactggttaagcaagcttcccaaggccatacagctaataagtgttaaacaatcaaacacagaactgactggctccaaagccagtgcccgccccactggccactccactaccctgcctctgccattgctccctatggcaacctggaaactgtcgggtacctgcccgtggtctgaagtgggcaggcagcagtcagctgggcctccagatatgggtggtgaagggaggatacaggtccaaaagaatatcttaggggaaatagtgcacatgcccagaaaacttttatagtctcagaacttagatgtgcggagaaatcgaattctggtcctggtttgcttttttagccagttgtgggccttggcagtcactgaacccctccttatttcttcatgaggaaattaacatgcctaggataggaaccgctgtgctgcagaccccagggtcaggcaagtctcaaagaagatgagagtcaccaagcgcctcaggaaagctgaagggcacagggtgcatgctgcagccatgacctcaacctctgtgagtctagaggtgaccatattggccacacataagctcaccttccccctacatttcataatccttccctcccatcagggccagcctttgattaaaattcccaagattatctgaggagctggcggtaccacccaccaatggagttcatctggctgcctttacaccagccaccttatggctgggtgggcactttcctattcttcggccaacacaacactttccaaactgtgtgacagttgtttatttacttgcctctaccataagaccatgagctttttgaaagcatggtctgtcttattcactgttaggtatcacagtgtattcacaatccctgacacatagtaggtgcttaataaatgtgtggttgaatgaataaatgaattaatgaatgaatgaatgaatgaatgggatcagggtgggccttcagtctctaaaagcatacccaagagccagccctctcagccgcaggtccgcacatggggaagccctagggaggttcaaggactagatcaggcccattttctcatcaggcagcccccaaacctctgccccaaagagcacatccaacatgctttcagcccacactccccacccacggtttactgcctgtaatcccagctactcgggaggccgaggcaggagaatcgcttgaactcgagaggcagaggttgcagtgagccgagaccaccccattgcactccagcctgagtgacaagagcaaaactctgtctcaaaaaaaaaaaaaaaagaataaccttgggctgtttggccaagttaatgcttaatgctttcctcatgctgcaagcgtttatctttgcccagattccagctccagggaggggaacattcctgcagaacagccaggcgttcccaggtcacctctgctctcactctcacttttctttttgcatgattaaatccggcctaaaaacacgtctcaggggcacatatctgtcactcataataatcctataaggtaggtactactgttagtcttattatatagttgagacacagagaggttaaataactagcccaaggtcacacagttaataagtagcagagctgggatttaagccaggcaatctggctccagagctcggtgcctttttttttctttgacagggtttggctctgtcacctgagctggagtgcagtggcacaatcactgctcactgcagtctcgacctcgctcaggtgatcctcccacctcaacctcccaagtagctgggactacaggcacgcaccaccatacccagctagagcccaagctcctaaccgcttctgctgtgcagtaacaagccacgctcagggcatgggcccaacttgagtttcttccatccacgggtccagactggaaaggaagcagcaatcttctcttcttttctcactgagtataaggcacccaattcatcttctggcagctgacctggaggacggagtggccctgattttccttttacaggtacaatcactgccaatactaaagcagtgcctgtccctacctgccagggcacccaggatccttgtctccagcagtaacttcatgaggtaggtggctctgggctgagacaggacttgcatattcctagatcccttgactgtgatgtcgaaggtcctctgcccccacccctccttctattccttgtaagcttcacttggcaattgtgtagtttatgtctggtacaggaatgaaggataaggtcaaatgaagctcaaagccaggagctagatagtcatgatatatcaaataaagttttggaacaagaacacaatgggattaatttgccaatcaaaacagcgcttggcaacttgactgtaagccaagatctctcatggcaagagcagctgaagggcctgtgattgtccagctcctggggagcctccctggcctaaccgtggtcagatgccagcatcacagcccccttagctgggtaaggattctcctagtgctacctctgggagagaaagaaaaaaatctagtctgagaatgacaccagcagacagcagctctgccaggtgtttgaagggtgaaggtcatgggactctgtttttactaccctggtcaccccaattgtttcggggtattttccatagacaagaggatgccccagtcagctgtcttccgtgattcaagacccagtctactgtcttctgtgattcaagacctgtttcctcttgtgccgatcagaggccttatttggaggcatcggcctttatgtggcatggaatgtaagccctctgagctttaacagagataggaagagtcctagaacatggcagggaagagggtcggaacctctgtcctccagcacagtagaaggatgctggcttgaatgtcaggagagcccagagtttctacctggatgcatggtttggggcatgtcaggtggcctttctgggtcccagtttcttcaactgtgacataaggaaggtagactagatcatcactaccaataaaaggtccctggtccttggggagcagtggttggagtgggagaagggcatcgcccagctattttccaacacatcgtaaaagatatcgcacaggacaactgaatgttgacttggtttgcttacctgtggaattctatccacttggtgtggtaaaacggattctcgccagagaactggaagtgtagatgggtggtgttacctatatccttgattgataagagaaggaaaatgaatttttcctcaataagtacaggttgtttgaaatgcaggctgggtgcagcgacgctgcatggtctccaggcgccttgtgtttctaaccctcttgctggacaacttggcctccacagggtagtccccagggaacccttggccatctccggcctgaaggaatcagggaagaccttccggacatgccaagtctccctgagaccctgctccatgagggctggctccctccccgacctggtcttttggttcaaagtcagtcctggagttatgtctgccaattgaccccaacctccagaaaactcagaaacagcctgtcctctcaggcctgcaaaccaggagcagccaggcccagccctgacttatgcaagtaactaccacagaaggcctgctgtgcagcctatgccgctcaacctggatcaagggcaatgtggagaaaccacattattgcttgactatagtgcagtattccaacaatcattttaaatgtgcacttgtgtaaccatgatcaccatcaagatgtaggatatttccatctctctttagagattcccttttgccctttgctagttaatccccaccccattccctggacccaggaaatcactgatctgctctcaatcacaatagctttgcttttccagaacgtcctgtaaatggaatcacacagtttgtacccttttaaatctagcttctttcacttagctttatgcattttagatgtatccatgttctggcatgtgtcagcaattgttcctttttcctgctgagtagtattccattggatggatatgcgacaatttgtttatccatttaccagttgatagatatttgggttgtttcagatttggggttattatgaataaagctgccatgaacatttgaatatttatgtggacatatgtttccatttctcttgagtaaacgcttgaagtttactgggtcctatagtaagtgtacatttgcctttaaagaacttgtcaaacggttttccaaagaaagtattttttgttctcaccagcaatctatgaggagttctacccggttttgtcagtctttttaattttagccattctaaagggagagaagtgatattccgttgtgttttaaatttgcatttctcccatgactaatgatgttaagcacattttcttgtacacatgtgccctttgtgtatcttcttttaaggatatctgttcagttctctcaccctaccttgaacctacacagggtggaatgtgcctatgacctcggaaggactttagggaagcaagccccactttccccagagaggattccagtacaaacggaaagctcttctgaatggcatactctttcataaccagatttcttaaaagaaaaaaatcattctgattctagcctgaagtgcagtagaattcaagagaatttaagagaaaagtttagaaggaaaaaatttttggcaagaaaaaagactctacttacttttttccctgggatccacaaaagaagaagttgtggcctcaggctacagtagtatggagccaaaatggactcaaaaaaaaaaaaaaaaaaccccagaataaatcagacatgaaaataactttccttccatcccgtatctcattttcttatttgacaaaaaaatagtaaagagcaactacaacaacaaagaagaggtgaaacaagaatgcaaaaaatgatgggaagatggcaacctccaaaatgtaataaattaatttcagcagctttaaacctaaagctatttgtaaagaatagctagaaaagaaacccactcaagacaaacttaggaatttcttctgagttcaaaacaatatcttaatgacctgaaaaattaccacggctttacaaagcacctcattcactccttaggagtgaattataccttgggtaattgaggttcgacattatagtgaagtgatgctagggtgtcacaagaggtgaaggacaatacaaagtttagtaactattgtgcacattcagaaattgagacacagaattactttcaagaaaaaagtaaatgggagggtcaggtgtccaggtcagggcaaaattcaagatggtgggagtgggagaaacattcctcaccatggccttgggcagaatctgcttatctccagacaaagctcagcaacagctaggagctgaatctgtaaggtccagcagtgactaaattcccacacaaatgtttccatctttccatcgtgcaagaaattctgaagccaacattgcccagctacagatcattggcctgaccacagaagccagggacagagctgcggttacatttattacataggattgttccatcctgagcagagtacatggcctggagagttttcagggcaattatgaaatttcggtcttccagaggacccttgccaggtctaggaatattttcagcagtggatgtttacaaagcaaaagcccccctacacacacattagcaaattaaataatgcatgttgtatgaaaaaccttgtgaggggcatagcaatacccgaggcttctttcttatgcacttgtgtgcccagtaccagccaatacatggaaagggctcaacaaacactgaactgagcaacagcttctagcccaggcatcatgggcgggaggcagatcctgactccaatcgggcctcctatggtagaatccctggttctctcccatggaaggtgccccacctcaattgcctgcagtcacccatctgctgatgtccttacccaaaagaaaatgaaataatttcttcttgtatgaaatatcaggaaccctcaaacctaggagttaactaggaatagaaaaatcacttgctgtaagaaataatattagaaatgtaatggctggacccagcttctccttctaactaattttaaggattgttttgctgctctaggaacttactgtcatacagaggacaaccccaagcctccctagtccatcaaccccttttcctggatacaggggctgaatcccttgatggatttgatcacttacctcaatttaatattcctcctcctaaaaccacagtatcatccccagattcttatcactggtgtgactacctcttctgaagacatgagaaaagctccagtttcagaaggtatgcttggagaatgtgctgcttctagcttatccaatatggaaccaatttcttcactcatagctaaagcatatcctttcctcacagcttatcatagagtggccaggtttaaatgagtcaatacaaataagactgctggaaaagtgcctggcacagagaaagcattcaatatatatcagcttttaaaaatgctgaagatcaatttgaatatatatgtatatataaagagatataaataggatcacacgtaaggaactgattctacttttaggaaactacactaagaaaatactaatagattccgataaatatttatacataaagaatatcatcagagaattgtttgtaatagggcaaataaaaccttgaaacagcatacattttcaacaatagtggaataagtaaataaattacggcactttttccaagtacgttttgaagaatttttgatgctatgagaaaatgataatgataagtgaagaaggatttaaaaccatttatgtaacatgatctcaattatacacactgtaaataagaatctatggaacaaatatctccataagaaaacattatgaagatatactccaaatgttatttgtgtttttttctaaataataatattctttattttttcagcacttttcaaattatactgaaatatattttcagcatacatacaatgagcatcccttaattctacaaaaagcaaaataaaatatgaaacagttaaaagtctattgcaacactataatttgaaggggaaaaaaaaggctaaggacatacagtaggccactgggcagacaaaggagatcattctgagcctgagtcctcagtatttggtgacccagtagagaggcaagagagggcataagctctaagctttccaccttgggcatagagatgaatgacggtgttatgaaccacaataggaaacgtaggaagaaaggaagaagaatgggttgggaggaaggaaaatacatttagtttggccttgctgagtctgtagtgcccaaagagtacccaggcaaagatgcccagtcgagagctggtgatgtgcaaagctaaaacttgtaggaagaaactgcagtaaaggtatgagggtgggcacagcagtgagtccttgaagccacgggcttcccttaggatgcccaagtgtcctaagtgttctaggaataagcgacagactctgatctcatggaggctcagacccctgggcttcagggctctattccttccacccctaaatgtgacctgaagtggcccctgcctccctcagtcctagaggctgctgctcaattacagtgaagaatctcttatagactcacggtgacttaaacgctatttggtgctgtggctgctatttcactgaagattacagggctctcagagttaaccacataaaacttgctgctatatatttgtaacctcctttttcaataacacatagggaaaccatcttgggtttcaggaccaaaaaagtgtgagtgcctggccaccaggaatgctataactgatcccgcttccctctttgatgtggccaaaaagaagctcagaaaaaaatctgtggcccatcttcagcagccttataagatcacatagtgtacatttttgggtgttaatattgacattcctatctttattgttttactgcccttattttctctttgtcccaagttcttcaactatgaatttttgccaactacttcaagttaaaaatgttcttccatgtcatgcccacccagtgattctgtttgtttctacctcttgaagttacacaaaataagtctagtctaccttctccacaacagcccttcaactctcatgtcatcctcccaatcttatttttataggcaccagagccccactttctttaacaattgctcgtggatatgagctcttaaagagtctttcctccatctagacttgactgatgctttatcttctctcgacctcttccctaaagcccctacgcagggcattcctccgctgcaacacagcaaccacttttaatagttgcttttcaagcttgcatcccccaccccaccatggggctggggactcctgaaaggagccacatgctgtctttgcctccaactagtacctagccagggggcctggggcataatacatgcttaataagtgtttgttgaggaagtgaataaatgaatgagcacatacctgtatacagtatacagcttcctcgctcatacacatgttaaagagagctaaagcctccgggaagggagaaaagagagggccctgttcagtccagttgtttgtggggctttccatctgacaactgttttccatggtctctcctgaacagcctcgaagcctcagccacctgggctggagcgtatctcacagctaaatccagagtaaatatacacaagacagaattggtcctatgaggacaactggggtgaatttccacagaaagaaaggatttcactgatcagaaatgtaagatgctagatgctccaattggtacttattctaacaacgctctcttctctaggttctaattacatgtcaggcattgttctgagctctttacaaatatcttatttaatcctcctaacaacctcataagataggcactatttttatccccattttattttcatatgaccctcttaacatttgaggcagctatcctaaaatgaagaaaggtgtagtgtttcagtctctgaataaaagaaaatgagctacattaggtccaatgtataaatggctgaagattgagtgccagcctttgatccaagatctggatctggcaggacacctcaccgctcactaacatttattgagcacccaccattgtgcccagggctaccctccatgagttcgctgacaggtggaggaagcacacacctggataaataaatcaccccgctgcagggcaagtgtattaggatcagggaggcagtacggtgctgagagcaagaaggagtaattatctctgactagatgagaaggcttcctggaggctgtgaagtctcagttccattttaatgaatggagcagcagagacagagcaggaggtgaggtgcaaaggtaccggcatccaaaagggcatttgggaactgttggtagcaggagaacgaaaagagaggaggaggtgggggatgagggatcatcactcaggtggtggccttggctgcaataccatgaagctgaaagtcactaacaggtatagtcatttctgttttagactctttgagcagctgtgtgcaggaggacagagagaccaagcagagagagaccagttaggaggctgctgtaataaaccaagcaagacacagtgtgcacgggaactaaggcacaggattctagggatatttaggaaacctgaagctttggactgatcgagaagctggcatagcagtgcaaggtgacaccccggcttctgacttagacaggagggcagccagtgaaatcagcaggctaggttggaagcaggcttccaggagtgagggcaagctgagactctctataagctcagaaaagccaaagagagcggcagaaattttcttcattatctcagaaaagttattcaaattcttgctcacacctccttttaataaaatatgatgcatttcattctgtcttcatagtgaaaaatcattgccctttgctgagaattaataaccttttgagttaataccctttgggtaggaaaattggtttcattaacacctccccttgcactttgtaggttcatcatctcttggtcctgctgtcccctgaaaccaaaagcccaagacatattatttgagaagcctgaatgattttattctcagaaagaatgatcagggtccttattaaatgggctgaattgagttcactgaccaaaaaggtcagagcggccttgtgtctgctcctcagccgcaggggagcccaccttctaccagaaggaagggatgagagcttcctctcccagctcttggcctgatccctaatcttggcagaaaagatgcgcccatcttgacatgccacgccatggaaacgcgccaccttgccaggctctaccaggcctcttggcagaggtgtccgtctctggtgcagcaccacagacaggacagctgtcgggctgcctggcagagttggatggccactcacccacccctccaaagtggctcatggtcatagtgaggctcccccagaagagaggctggaaggcccttcggtcaaacaacagaaggtgatatgttaaaaacaatcttcctctccttttccacctcccacggccttggaaagcctccgaatggacattcctctactcttttctgttgcaaactctttagcagcagcctggcatctggagcatgaagccagactcttcagcatcccagggcccttcatgctctggcctgcctggccttgtctcctgcctctttatgtatgcaaggttgcatgcctgtgagcctcgactcatgttgctccctcagccacccctgacccccgcaacaaaacacacacacacactcaatcatcctgcaaggccagggtcaacatcacattccctgaaaagccctccctgaatgccacaccctccctcaagcggcatccactactccctctgtgattcccccaaccctgccccacactcacccctgtaccttgtccctgcaaggcttacctgatataatctgtctgcacgtgaggacaagttgagcctcaggagctaatacatagtgtcacccagctaagatgctctattcactgtctctctaggtagggatgtagggatgaaattggagacagcgacacctgggttcaaatcctgtctctatcagtttttggcagtgtgatttggggtgaggtgcttatccaccgagcctccaaaactgccttgcctatattccttatgacctagagggtgactctagtgcttgattggctggtcactgggataacaggctagaggatctggtgatctcaacctcctaggccaccctccatcacttggccattccactggaaacagtctttggggctccccacagttgtggctcattctgatctccagcatctcccagtaacctctttggcttgtgcttgtagaagcagcctttgagaagacggagggcttcagatgaagcagatgccaggctaagcaccgtccccaatcttatattgcagagccatttggaagaagagctcaagctgttgaaacaaacaaaacgctgcatgagatgaagaccagattcctgctctttggagaaaccaatcagggctgtcagattcgaatcaatcatccggacacgttacaggagtgcggcttcaactcctccctgcctctggtgatgataatccacgggtggtcggtaggaaatgctgacatgccgtttttctctccgatttcacattttctttttttctttctagcgtgttcatgttcataaaaagatagggagctggtgataacaactccatgcataatgtttatgaagcacgttctaattttccaagtgcttccccacgcattagctcattgattatctcgattccatgagtaagcagcgtgagaaaatataattatctccatgtttcagatagaatgtggctcaaagaggccacacagcccattggggcccacaagggattgcagtccgtgcaggaagaagcctccttcctgaatctgcatgttacacacagggggacataggaagccagaagaatggactcccaatgaaatgttgtatcagcaaggtttaagttcagcagcaaataacagagactcaaattagcagtggttcaaagaaaacagaaatttgttttcatttcgcaaaatattccaaagatatatgtaacccaaaaaccggctagacagctttggtcagtgaagtcctccaggccccagctccttccagttcactgctctgccatcttggggtgtgaccctcatcctcatagtccaagatggcagctagaggactagccatcacatctttattccaggaagcacaatgtagtaaaagatgtacacaaaggagtcaaaagcaaggtttttacaagagctttattgagatataatttacaaagcgtacaacttatccatttcaagtttacacaattcaatggcttttggtataaaagagtgttttttaggtgtcccagagcctctgtgaaattctcccagttacatccattgaccatatctaagtggaaaggtggctgagaagtataggctgttaaagaaaatgtagccttttagtgatgctaaaaattctattactatggaagaaggagagaaaggatattattggcaatagatgaagtcaaaccatgtgagaacttgtctccaagaggaaatcctgcctccccagtgtctagcccacccatgatttcaagcatagttcacaatttgaaccctccagagattttggtgtctacttcatgacttagaaacctttccctgccatctagttgaaaagtactcttgaaacccccatctatcctatcctggagaggaaaatgaacccttccatcacctgcctcccctcacctctccctgcctcccacatgacgttccagcaacatctaactgcttgtattaatagcttcctccacaccccatgcacctgccttctctcttcttggtcttggcttagtctgtgccctctgcctagagtgttctttctcctcctcttttcctgattaactcccacttttcctgtaagacacagtcccaaacaggtcctcctgcaagccattgctgccttcatcccagcactcccacagggcattcagtgtcaccgggagttcccgtgtggccctgagcactctccaccactgctctgcagctcggctttctaattgtctgtgtttctctgctctgctaggccgtaagcttctccctgggaaggattctgtcctattctcatctaatagactactgtgggccactatccattatgtaatcagtgctccaaaatgtttgttaactgaattaaagatcaaatgaatgagtcttgtttataaatgcaacatgaggaccacaggagcctcttaccaggatgcttttaccaaaatgcactcttcattatcaaaattctgtccaacctagtatatgtctcctcctcctattactgctaatattaataacaacaacaactgcagtaacgaacatttgttgagtccttactatgttccagacactgctctaagcgctttagatctattaacgtatttctccttcataacaatcctggaaggagagcattattaaccccattttccaggtgagaaaactgaagcacagagaaaaataacttgcccaaggttactcagctagaaactaggagagcctagatttgaacccaagatgctccatgcacatagaaggcacttggtaaatatctagtaaaggaatgcatcttaccctggaatggcatgacaggccctcaggggagctctccagcttcacccgctattgtcaagccttcacctcctattgtctagccttcacctcaccaactttcctcctctcccctgacataccacactgccctttcatgattctgtgactctgcacgcactgttccttctgcctgggatgccctagacctcaccactaccacattcacctgcccacctgtattctttttgtttctttgttttgttttgttgcgatagggtctcactctatcgcccaggctggagggcagtgtgatctcagctcactgcagactcgacctcctaggctcaagccatcctcccacctcagactctggagtagctggggctagaggcatgcaccatcacacctggctaacttttgtattttttgtggagatggggtttcaccatgttccccaggctggtcttaaactcctaggctcaagcaatccccctgccttggcctcccaaagtgctaggagtacaagcatgagccaccgcgcccagcccccacccatattcgtcttactggactcctattgatcctttaaggcccagcacaagtgtctttgctctgtaaatccattaggagtgccctgcaaattggttctcaaacaaggcacacacacagaacaattctctgtaaacattttttacctcctactatgtcaggctcacagtgcccatcccgctaagtccagcataacgcagtctgcagggcagagccagtgctatgggtttaggggtgaggtggggcagctgcacgcagaaggaggcgtacaaatggccctaagggacaggtcagcttccaggacctgatttacacatatttctccactgttttaaggtttagaaagtccctaagaggaaaaacattaagataattgttctcgggagggggcgggggggaacgtttggaaaaatctggagacatttttggttttcaaaacctggggcagtgggtgggtgttgctggcatctagttggtgaaggccaggaatgctgctaaacatcgtacaatgcccaagacagcccccacaacaaggaattctctggcccaaaatgtcaactgtgcatggctgagaaacgtcatagcaagcccttcctccagcattatccaggagctggagaaggaagaagggtgagcggggagaaaggaaactagtgcgaccctccctctgtcccctcctcaggtggacggcgtgctagaaaactggatctggcagatggtggccgcgctgaagtctcagccggcccagccagtgaacgtggggctggtggactggatcaccctggcccacgaccactacaccatcgccgtccgcaacacccgccttgtgggcaaggaggtcgcggctcttctccggtggctggaggtaccgacctgccccatccttccttcacctcccttccctcctttcccttcctctgagagtgaatgaattaagctggtctccaacagcagcccaggcaggagaagcactaatgctcagctcacaggaatgagaaggcacaggactgacaccagaccccagggctctctcagacgcctcccttgaacccctgtttcacagaaccaccttctcccctctcctcatccactattcctcttatgctcacccccttagacatgctaagggatacaaacacaccctgtaagctatcaagcccccacacaaatgagtgggatcacttctgtagggcattagtgtccaggggaagacaggcaggcaggaggctgcagctacaaggcgaggtcacaggtacctgagtcccaggagagtcagccagttgagagattagtttcagtttggagtgtgaataaggcacctcatttctgagcaggcacgaagaacagggtgggcgccacaacacactggaccgcaaaaggctttcatccaggcagctcttctcctgcccccatcccgctgctgtcttccaggaatctgtgcaactctctcgaagccatgttcacctaattgggtacagcctgggtgcacacgtgtcaggatttgccggcagttccatcggtggaacgcacaagattgggagaatcacaggtaaccatgcctaataactcacacactgatctccactccataggggcatccaacaaggacctgcttttccaacaggctcatcattaaaacaccccaacacttattgtgagggatcagcgctcatgagaggacttgtgacattctttcaaacatgaccaatgtcatggattgctgttccagagaattcagagcaaattatagcctcaattcctattgcctgccctcaaacgaatacctcatgttggtgtgccaaatgggactaaaagatggacttttctagcaagaaagagtcttattttttaatctataatttacaaagtccaatttgggcgagttaaggagtgaagaatcacagccttaatttgctgtaattcatcagccacgttagtgcctccgtttccacatttgtgaaacgtgtatgttttagtctgggacccatttgggttgagggagggagctctgcatccaaaacttcaccccaaactccatacacactcagaaatggcccaagatatccaggaaggtctgcacatgtgcgaggaaggccttcctcccaggcttccgtgacaccaaccccacccttcacactgccaggcattggatccaaaaacacacagctggtgagattgttccccagtccaagtctgaggcccactgataccacaggtataaaacccgggaggcacaggcaaggcctttctcggtgtggaagccccagagtgccccagcctctcctcctgctgatcccttcccatagctctaggcttcaacgacacaaggtttttctctacttcacaaacacaccatggcctcttgtgcctctgggcctttgcatgcactgtttgctttgcccaccactcagccactcaaactcctcctcatccttcaagacccagcaggctcaaacaatatttcacaggccaaaacaaagagcattttacatgaataacaaagttgttgcgaatttatattttatcaagtgaggacatttttttttcttttgagacggagtttcgctcttgttgcccaggctggtgtgcaatggcatgatgttaactcaccgcaacctctgcctcccgggttcaagcgattctcctgccttagcctcccgagtagctaagccacacccagctaatttttttgtatttttagtagatacggggtttcaccatattggtcaggctggtctcgaactcccaacctcaggtgatccaccatctcggcctcccaaagtgctgggattacagacgtgagccaccacggccggccatgaggacatttttacaaacaactactatctgctattaccattcctttataaaagaatttcttttaacttgagaaagaatggggataatggcagacgtttcaacagaatacacttgtgattatgacagctcatttcctcatgcttttttgttcatcttgctggtcactggggaagcctggtcacagaccggcgtgtggcccccatggatttaacccacaatagcctggggatgagcagccctcccactgaagaagcagggccagaggtcagagtccacacccagcgaggcagcctcaggcccaaggcccagaaccctctccatccaactggtgctgtgggtcacattaggcgtatcagctgggcccacccacctcagagacagcaggaaagctcaacaccctcttgccgtcttgtcatcagcctgataaagaaaggaacagaaatcatatcaggacatttttctttttctctttcttttttttttttttttttgagacagagtctcattctgttgcccaggctacagggcagtggcatgatcttggctcactgcagcctccacctccctggttcaagtgattctcctgcctcagcttcccaagtagctgggactacaggtgtccaccaccacgcccggctaatttttgtatttttagtagaaacggggttttgtcatgttgcccaggttggtctcgaactcctgacctcaggtgatctgccagactcagcctcccaaagtgctgggattaccaggcgtgagccaccgcacccggccaaggacatttttcatttgtcactgtgggtagggctggagaacgggtgatctcacggctcactctcaattccttcgccccccaagtagggaaggaaaccagggctggggtgggacaccagagggtcctccagagaaggcagaggaagaaagacagggaaaaaaaacttggagcgcataaatactccccaaccacagtcagccaggaggtgccaccttacttcatgcacagacatctgtcacttgaggcccagagctttgagctcccctacttgcacacaacattttcacatacagtgaaatgtcccgcggctgttctggacgttctaatgaaatgacatcttaggagaaaaatcagcttgtttagggaaattattgacaacatacaggcaaataaatttcattctttgataggtaggttggaactacttataatgagcatattggctttttttgtatgcaaacagatggtcttgaaagtagcttgagaggtactcaaaactgatttgctcaagtaaatcaagcattccctcataaccttattcctttttttttttctttttgagacaggatctccctctgttgcctagactgggtcacagtggtgcagtcatgattcgctgctacctcaaactcctgggctcaagcaaccctcctgccacagcctcctgagtggctgaaactacaggcacaggccaccacatccacataacttttgtattcttttttattttagagatggggtttcactatgctgcccaggcctcctggcctcaggcatcataaccttgtgtctaccactggagagtttccgcaaacctctttccccgctagctaatgcccagcctgtgtgtcattgttagcaccatgaactactgtggttttactgagaaaaggttacggcgaggttttctagggaaggatcagccctacgtgtttcttcttcctgctagttcactgatgtataataatatccaaaagctaaaaagcacatctctcttcccctctccttgctcctgcgtaacccttacccctgctttcccattagggctggatgccgcgggacctttgtttgagggaagtgcccccagcaatcgtctttctccagatgatgccaattttgtggatgccattcatacctttacccgggagcacatgggcctgagcgtgggcatcaaacagcccataggacactatgacttctatcccaacgggggctccttccagcctggctgccacttcctagagctctacagacatattgcccagcacggcttcaatggtgagaatgaagtcatgggccgggagcaccggcctacatttcaatggggcctctggaattcagcggaatctaccaacatacgggactcagggaagagttcagcaaggataggggcccagggtgtatggtcaccaagcccacccagagagaaggaatgctggagtgggcaagggtgcctgtcccccagcaggccactcctggcaacaggccaacgccacgccctgaggatgaacagagggcaccaacaggaacctccacttctcactccagccccatgtgcccaggcaaaccctctcccggggtaccagctcttctgagagttgagtccggatgcctgtggttattcttccttctctggaagtttcctcgttccagggaccgcctctattctgaaccacatgaggatccacttaataggttgtggcttctcgatgaaggtctcactcttcatcccaccccatcagtttaattgaggaaattactcagactgtcccaatgggaaattcagcttcaaatatgtccttgttatgccctttacctcagggcagacacggaatgtgctgttatctgggttgtgatcccacttctctctcaagtcctaaaaccgaaatattaacatgcacattgataatatgcacaggattaaagaacaaaaatgtcagcagaagcagcacattccacgggcattccaaatttcttgctattataaaatatcaccctgattaagacatgccaatcactccaagccaagtagagccctccacccctacaagccacctcaccctgatcatccaacactcagctgagaacacactctcttgtgatgacaccatcctcgcctctccaatatcagagtgaccttcacactccgcatcatctcattttggctggactcccagccttccaccgcccaggcctgagagagccttctgggaacttgcccttatcattgcttacatccctcccacctcccccactccaacacctcccctccagccaccagcagttcccagctgaccatccagccctcatcccaaacttcaccccacccaacaaagctcagtacagaccatcaggatggtgtatgggctttattttcctggtaaacacatttctgaccaatcattaccatttgctcttttgtgctaaacttcaaagtgacggaaccaaatgtaggtattcagcttgcccaagcttgggctggtttttctcagtcgtcttcacccctgtgtttgaaagggttcctggggtagttctcccagccacagctgcacaggggctcactggataactgcagattcaccttcactacctcctgggtggatgagcttgggtgagttagtggatgtcttggaagctcagttttctcgtcgaaaacagcggcctcaaggagctgcagtgaagatgcaggcagtggaggagcggagggcacgcagcatacacagcctcctagtaggtgcccaaggaatgcagccgctagcgtccaaggactgcagtgactaatcttggcatttagcctccagacaactggggaaatagcttttggcaaagtcacctgtgctgtccctttacagcaagtgtttcacatatcttgtggtggttcctcaacatcccttgagaattatttaccacagattaggttaaaatttggagagttaaaaaaaaaaaaaaaaggtcaaatccttagtagatcaacctcataagcttgttttgggggtgaggcctgtccagatctggttactgggcaatgtccttccaaatccaattcttgtgctggtttcacaggcctgttacctaagggatgcaatggctgctttgggacacaaggtagacattgtcgagaggcctcagcgttctccagaccatcagagtgccacgggattgtctcgtgtatctagttatattaagagaagaggagtcccacgcaagcctgttccagaaaacccaccctccaggcatcagcacctgggcattttaaattatctcatttgagatagaaatatttctataataaattacactaacctgctctcttaatgaatttgatctttccttagtgacttgggtgcctattaaaatgcctactgtaacacgctgtcataaaggagtgctctcaggatcccggcagtgcccgggattttgcctgacactacatgactcagagggtctacttcccttagtttcagtcagttgtcacatcctgctcttgtcaaggggtctgccttgacaagcccacccttgcctgttctgtgtgctactgctaacctcctgtgggatgagaaccaaggtgatcctctgagttgaggctgctttgggttaaggggtgataacgtccttcttgccctgtgttccagccatcacccagaccataaaatgctcccacgagcgatcggtgcaccttttcatcgactccttgctgcacgccggcacgcagagcatggcctacccgtgtggtgacatgaacagcttcagccagggcctgtgcctgagctgcaagaagggccgctgcaacacgctgggctaccacgtccgccaggagccgcggagcaagagcaagaggctcttcctcgtaacgcgagcccagtcccccttcaaaggtgagtgtggagctggggagccttcagaagggcaggatgcagtcccctgtccaaagggctcagaagtccccttggcttctttcctggaggtgcttcagctctgctgttgcggtgtttctgaaactgtgcaggctccagacagcaacgttgacacagcctttctcctgtcccaagagtgtggccaccttgccgccaagggctaggaggagaaacctcgaccatttctgaaacgatccttgggttggcctctctgtccattttcatgctcccctagaatgttagatctgcaaggccacacgcagtttacctgggccacccggatctcactgtacagggacgtcaggcatctccaaacacatcctagaacccagttctttcatctctgttcacggcccccatgaaaaaactggattctagactgcatttcatttcttctaagatttgaatcagctgatctgataggacatgatattcaccctgaggcactcccaagacaagattcagcttccagtgtttcccagccagatttttaaccacagggcagaaagtgggcccagcagctctctggattgaatggaagattgtagaaagggctgtgaccacgtcttaggctgaggccagcagagaaatactccactgaaacctaaatcctcctggagggaacaagccgacatctgttgccattaacatttctgcctaatccctaaaacagctcgggagtgctttagcatctcactgtttcatcgcacagcctctccagtgctgggagaagaaatctgatgtgctgtttgactattaccaggcagatatttgtatctggagaatgtaaacaggccgtcaccaccactttgcttttagggagcattttacacaaaacattcgatgcagccatcagctccttgtggttctcccaacagccaggcgaggatggcagggctggttattatcccattctaaagaggagcaaagcaaaactcgcgtggggtgcccacagctgtgcagctgtaaggagacctggaacagggccacctgactctcagtcgtgagttcaaactcccaggcagcttatttttgaggcacaggatttctctgtgtatcggaacttttgctctccagtcaggtttccctcccttgagtcaagcaggtctaggaatgacagaaatttcttgtgggtgcgcacagcatctgaagagtagttggtgtgagggcaggatctttcgggccaaagggtccctccactggctgtcccttagggctggaggagatggcagcacgcaggtgagggaatttggaagtcctcctgcttcggggagctcctccgctgcaggctgccaattaaagactcatttgggtgaaccagctcctcttttaaatggcaatgccgcctggagtgattaagcctcttagataatcacatttcactggatctattttaatcatggaatgttcgcatcatgaggagcagttcaaccctgccgtttttcagatggggaaaccaaggcctaggcagggaaaattatttgtccagagacaaaaagctcttggcagacctggcatttagacctggggcctccaaagagtgagcagagtgatttttaaaaactcctaaacctgccgctggtctgtttatggggataagaggggtatggaagagaagggtgggggaattgctttctccttgaaatcacctgggaagaaaagaggtcatttttcatcttccagggctaaactgggcacttcatctgatttggcccagccctaggacatctactggggacttggagattgggaagggaagcatcaagagaaggaagaaggggaaacaagagcagtaggcatgaaccaggctgagcttctcccatttgccaacggccccactgcccgaccaccatacactgctgatgtgaactcacaaggatccctttgttattaaggaagaccaaagaattgcattagggcatcattatctcacccaccagctaccaaaaccaacgtactcatccatccgtatattcttcaaacacagtcctaccctcagggggctcggtggagactaacccacgatgagttccttggagaccacatttgtgtctctttattaccgactacttatcttgctcctgggacatctagtgaggaggaggttaagggcaggactgactgaaactggtcaggaaccgaaagccccaggctggagggaggttccagtgccgagctgtggcttgtaccacaccctctccttttctttcttactttttttttttttttttttttttttttttgagaccaagtctcactctgtcgcccaggctggagtgcagtggcgtgatcttggctcactgcaacctccacctgcagggttcaagtgattctcctgcctcagcctcctaagtagctgggattaccagcgtgcaccaccacgcccagctaatttttgtatttttagtagagacggggtttcgccacgttggccaggctggtctcgaactcctgacctcaagtgatccacccgcctctgcctcccaaagtgctggaattacaggtgtgagctactgtgcccagcccataccctctccttttctgctcccccacaaattctccccagttctcctgcagcccctccagtgagcaaagtcctcacccgctcaacccagtttcacttgctagtggaaagtaacgtctctgtggtcacatcaagccaccctgggcatggctggaaagtgcactcatgcgagactcgcctgtttgaagcagctgctcatgggatactgctatctatataatgttatgttatccaatgtggtagccacaaaccatatgtggctatttaaatccaaatctaagttactttaaattaaatggaaccaaaaattcagtttgtcagttacacaagccacctttcaagtgctcaaatacgtggctgctgcatcagagaacacagaaataaaacatttccatcgccgcggagagttctactggtgatgctgggatgaagattttaatctaggccaccacagctacaccactccctgcccatcacaaccgtgatcataactgacttgggcccccaaagcctattccaccagccacctccagcattttgatgaatacatattacactcttaaaagagcagacttgcccaaccccggcctacagagaaaccacaatccccatttttcaaaaacatctaattgtacaatgtattttagtgcagggtctggttgggcaagataaacatcgcctcctgattgtagtttcacaaaggatgactaacttggcctgaattcctctggctcccaagatatttcgacaaaaccatcaaaggatctattttctacaactgtacaccagaactcgggtaaacactgcgagagtacagcagtagcaagagagaattgggaaacagtcgccgagtgaaacgaggcaatttatcgcccacttccattctcactctctcacaaggaacctttaccagtttgggagcctgtttttaagggagggggagggaaacggatctgtacggagtaaatcctgtttcgccagtgcctcctctcatttcatcctcagagcagtcagatgaggtaaccgggtggagaggagggaaggggccctcggggggcactctgcccgggcactgggctggatgtgttgtacacgttagctcatttcccatttgatcctcacagcaacccccagacagggagagacacacgcacacacagagacacggtcagggcaaagtcacacaatttgtgagcaggaagcatcgcattgaatcccagcctttgctccttttactgcacttggcctcggcgtaggaccaaggccctcgactgagtgcctctccctcctgcggcattccagctgcatgtcccgcctcctcctctctgccctccttccacactccctcttccatatctcctccactcttctcctgtcttccccgtgctgcccttccccctcccccgccccctcacccacggccctgcactccaggctccctgggtcgggggtgaggggcacttgcttctgctgctcgccgcagccatgagagcacccttccttgctggtcggcgagatgacgagccaagctgtgactcgggcagtcggtctgtttttggcttctctcagctacttctttggaaggtttccactgacttcagggagagttccagaagaagcagggggtgtcgtagacagcacaggcttggggttgggataaggagtgtcaagaatcttggccttgctgccaaccacttatgtgaccttgacctaatgacaaaacctatgtgggcaccaaggaagaggtgtgaatagacatggggatgaaccactagcacgggacctgcaggtgggtctcaaaacctgccccggtcagttcagtgtggaggaaggaagtagatgacacccgattaacaccttctggaaggcaggcattttttcatcgccatcttatcacatttttacagcgacactacaagaaagttcctaacctgcattcacagatgaggaaatcgagccccagaaaggctatgtggtctccagatcacaaggaagcaggcatcagagaggactcaaaccctgggctctgtctatccgttgagggcccttattccacccacagcacaaagagctggcaacaggacgccagtcagcacaacagtcctggccgcacacacacactcccccatctccaccgctcgcaggcagcaacagacccccacagaaggggcaggcattggccaggcacggtggctcacacctgaagtcccaacactctgggaggccaaggtgggaggatctcttgagcccaggcgttccaaaccagctcgggcaacaaagcaagacgccatctctacaaaagaattaaaaattagccaggtagagtggtgcatgtctgtagtcccagctacttgggagactgcaggagggttgcttgagcctagaaggtcaaggctgcagtgagacatgatggtgccactgcactctagcctgggtgacagagtgagaccctgtttcaaaaaaattcaatttaattttaaaaagaaaggggcaagcatccagtctcgtgacatcagggcatcacttaaatgggactgtactgttaaaccaaaatgtggccacgaaaataccgacacatacaggtgttacatacaaaggctaacaaaatcactttccctttttcttcttcactcgcgcctgctctgagttaattcactttcattcagccttgccggcactggctgccttctttgggcagcaacaccctggacaacaccatcctgggcccctcaatcctatgatcttcagcttccctctgagttagaggcatttcgggtctaggaacaagccctctgggcttggagaaaacacaagaggtctcattcctaccatctccatgagagaaatccatacacggagggggaaaaaaaaaaagccctctttgatgtttgcaaagagagtcataatatctcccagagccaacatctaatgattcagaatcattgtttctgggaggcaaaaaatagaaagaaatatattctcttttcataaattcaataacctgatcttgtgaaaagaaaatgtaagttttttcttttttaaatggcaccagcagggtccacacagggttacaatccaggatttttcctagtgaggagactctccggaggcctcagatttctgctgtaaatggagtttccctgatcacattcgaggtcaatggactatttcatgcctttcatctttaagacctgaaataatttctcctgccagaacttgggagtatgttgtactggtctctttgcagtcacaggattaaaagcgttttccccaaatcattttttaacgtccttattaaacatgtcataaggtaaagtgggcaaaatcactcacacttttttccttaatctttgtaatagcatcattgtgctgggggtggcagggtagaatgataggcaaattgtcttttcttttcttccttttttttttttttaaagaaaaacacttacaaaaatccaaataagagtaaataattaggactgggtgtggcggctcacacctgtaatcctaacactttgggaggccaatgcaggaggattgtgtgagcccaggattttgagaccagcctgagcaacatacagacctcatctctacaaaagaggaggaggaagaagaagaaggaggaggaggaggaactagttctcagcaggaaccagttctcagcctgaaagactcatttatatttaaagttaatttcatttaaagatttctccaaaaaagggcagtcatgcaagattctccgtgcagacatggagtttctcaggagtgctcctgcaaactcaagggtgcctcagccatcagcgcaggttccgtccccagcccgttccctggtgagcctcagaaagcatgctggcttggctcttcacagcccacacaccttggcacgttccatcctcctgcagagatgctactctagggacacatgttgtagaaataggcaattctcattcataggatgtgcaagctaaaggggcctttgcatcctctaaagctgactcctgggttttacacaagcactttctcctaaaatcgtggaaccatagcatctcaaatgcagaaatcaatctcctcctactcacaatttaagaaaaagggaagaaaaagtgtatcgccaagacaagggcatgaaacattttccccggctcaggggccagacgtttaggggacaggagagcgtcctttgtggcagcgatagcccaggatggatgtcaaggggtcagagctggcaggcagtttgtcttggtggcttcattagaagcaggtgttgaaccacagcttttcttcaggggctgttggccatttgctttcaagggacagactcagcttgcagaacgaaaacagcaggcgatatagaaagagtcttttggaattagacacacctggctttgaatcctgctccactgaactatctggataactataggtctccaaacctccacttccttatttatattcaccaggataattaacactgtgtcggtctaggtctcagcagaaaacaggactccactggcctggatcagctgaagaaagcaagtgggactccttacaacagggtgggcagtgttaaggaaaccaacaaggactgaggcgactccctgcctctaacagcaggacagatggagccagggaagggactagctgtgggaaaaccagagccatggaggaggggctgctgataatcgtgggagggcagtggctgcacagtcaggctccagagcacgggggagcagagggaaaatgcccctgcctctctcttctccgccctgatctcctgctagtgcctctccctccagtggtcacacccaaccagaaaccaatcgacaagggagcccggcagatgcagcctaagggatcccatcctggggctcagagctgagcagtggagggcagagtggatgaggacagcaggggtagagggagcagatgggcactacccagcagggcatcattatttagcctgttatgaggagaaagatgatgtatgtaaaacacttagtatgtagcttaacacatagtaggatctcaacaaatgggagctattttatcatccttggtgtctttaatgcagttatatatgcatgttgttcttcactcagagagcctgtgaagtactccttggaggcctagtgtgtgctgtgcccttgtggagatacaagcagtagtgtggttccgagatggcctttggggagataagacatggacacagatgcccgtgagacaaggcagaggccacaaaggaccacaacttctagttctcttttttttccttttcaatacttgtaaaagctctttctttttccaataggattccaattagaatacacgtttactgcagaaaatgtgaaacttgcagaagaacataaagaagagaataaaaatgaccactctgccctctccagagataagcactgttaatatttttgtgaattcccctacttctatgcattctgttttgcagagttggtctcatactagacagtttcgtagcctgtgtttttttgtttttgttttctttttcacttctttcataagcattgaaatcattccaaaaatagcttttaatggcttcataagagtaaatcacatgtttgggacaatgaactgagaatcagcagactaacttctaattgcagcttcatcagttattagccacgtgacaacgacaagttacttaactttttctgagcctcagcttcttcctttgcaaaatgatgtgggagggaagataaagtaggcatcttataaagttgttgtaaagagaaatgagataattatatgaaactattagctcaatgtctgggacatagtgaagcgctaaatagtagttatcataattgctgttattagttcatagccatccctctattactgaatatttcaggtgttttcagtgtatttttacacaaatgttaggacattttttaactgaaatctctttccacaattcaactttttccttagaatatataaagctagtggtcctgggtataaacatgtaaaagttcaagttctggagtcaggttgtgtgagccgacgccccagccatatacgacttactagctgtgtgatccgagaaaagtcacttaacttctctgtccttcagtttcctcttctctaaaatgagataataatcgtatcatttgtttgagggtcaagtgagatgacagaagcaaagcacttggcaaaaaccaaaatagatattagccaatgttattatcatgcatactgccattatatgctctttttttttttttttttttttttgagatgtagtcttgctctgtcacccaggctggagcgcagtggcgcgatctcagctcactgtaagctccacctcctgggttcacaccattctcctgcctcagcctccccagcagctgagactacaggcacccaccaccacgccccgctaattttttgtatttttagtagagaacggggtttcaccgtgttagccaggatggtctcgatctgttgacctcgtgatccgcccccctcagcctcccaaagtgctgggattacaggcgtgagccaccgcgcccggcctcattatatgcttttaattgcatttagcactgggctaaacacacagtagacttccaattaatacatatcatattaaactgaattgaagatcggttactatgagtgttccttgagaactctggaaggtaccctgactattcataaggaggttaacccaaggccagtcctcccacatcttaccccctcccccactttggaaagaactttctttttgtctcagtttgacttacaagactgttcatttgttacagagtttccttgacaaacccccaaatagtgtaagacctctccagaggcttctgggatgacaggctaagtccctctctccagtggtgccacccaagagtttagggacccttcccaggacttgcgcctatagctcttttctttttttttttttcttttttgagacagactcttgccctgtcaccaggctggagtgcagtggtgcaatctcagctcactgcaacctctgcctcctgggttcaagcaattctcctacctcagcctcccgagtagctgggactacaggcgcccaccaccacacccagctaatttttgtatttttagtagagacggggtttcaccatgttggccaggatggtctcaatctcttgaccttgtgatccacctgcctcggctcccaaagtgctgggattacaggtgcaagccaccgtgcccagccagctgttttcttacttctcacccacagcctcttttgctgtcttgagggcatcacattgctttattccaggcttgggatttgagcatctattgtattacagcaggggtccccaacccccgagctacagacagtgggcaattaggatccgggcctcacagccagaggtgagcagtgggcgagcaagcataaccgcctgagctccacctgtcagagcagtggcaacataagatcctcatagcagcgcaaaccctattgtgaactattatgagaatctaatgcctgatgatctgaggtggaacagtttcatcctgaagctatcccccctgcccccacactggtccatggaaaaactgtcttccatgaaacccatccctgatgcccaaaatgttggggactgctggtgtatagggtactgtagtaggcaccagatactacctgagctagctcttatctaaagcagaaaactcttggccaataaatcaactcttagtatttggctcttgttgtgaacataaataaacatttggtgcttcagagataacccctttagtaaacccaagagatatccccactcacatgagcaaattatatacaaaatccaaatgggaagattagtttatgccaaggataggattgtgtaatctagaaacttggcaatacaggcataccaggacactggagctggcagtaccctctgctgggcttgctgtatagctaccaacctccggtagaaatagtaccatttaggagttagtagattgattgatgaagagatgaatttccacatgctgggtacaagggatggatagtcaagtggctagttaccgtgatctaattttttgaaattctcctatgacaaagcttgaaactcaggtcttgaatcctcagaggtgatttccatgttgttagatgttaatgtgcattgtgatacgtcacccccaagcaaaccatcagttcattcatcccacaaaagtttattaagcacctactgtgtgcaaggcactgttaggattcgtggattaaccattgtctaaacaggaaaggcaaatacgcagcaagtattccataaatcccttatccttttcccaaaccagacactcatcttgccccaagcccggacatggcctcagaatccatctcaacagagggatccaggcagttcttatcaatcaaatggaattaacatgccgtattaaacctgtttgcaatcttggagctagaacactgtagatgcttagagaatatggagacaagagagctcaggaggtcaaggctgcagtaagccaaggtggcactgcacccaagcctcagtgacagagagagaccctgtctcaaaaaaaaaaaaaaaaaaaaaaacccaaaacaaaaacaaaagccaatcctggtctggaagctggaccctgcatccaaccctggggaatccactggggagaatatggagacaagagaaagaagtccaagcagtaatcagccattttggaatgaggtaggataattttcaaaacagctccatagctgtgccaccatgcaagctaagcaaacaccatctagggtgagggaagctaagagcacagatgctgctatttgaccttaagcaagtcactttcatctatctgagctccagttttcttatttataaaatgggggtgataataacaatgcctacatcatagcgttgtgatgagtaaatgagatgatgcaatgcattcaacaatgcaaaaatgtattatcatttaacgatgccatgtatttaacaacgcctgtctaatagtaagccatttgctttcagtaactaaatgtagactgtattaggtaaagaagaataaaataaattatcctcttccaaccaacccatcagtaggaggacattccagacatgtgataaaacagaaagattttaggcccaagttctagaactacagttatcttatttccattaggaaatacacttattgaaactcacagagcttgtttccaggaactgccccggaatataaacgcttggtcactagggcatatatttcttggctggagctctgccccaccctgaaacacaccccatcagagagcagcaataatgtaaaatggcaatgtgttctctcttgccttgtcaaatgcctaaaaggcatatattctttgatttacgaaaggagaccctcaccaaacacatgaaatattacagcagaacttgcatctagaactcagtgagaatgccagatgtcctatactcctattctgccatcttttgttggtgaatatttatcattattacattacaaacacaatataagtcctgaatagacatttaggaaatacagagaaggaaaaacaagaagataaaccaccatttaggagcaaaaaaatgttgttaacatattaatatctatccaaactcttccctctgcatgtttaaattttaagaaataagagatttttaaatttaaaatcactgcttaaattatctctctctttctctctctgtctctctctctagtttatcattaccagttcaagatccagttcatcaaccaaactgagacaccaatacaaacaacttttaccatgtcactactcggaacaaaagagaaaatgcagaaaattcccatcactctgtgagtaggaggtgtagccccctagggtgatgacacacttatttttcttgcagaacataaatggactctgtaattttcaagagtcaggccagctacaactactttattccagacgcaaaccaaaaactatctgagacacatgtcaatcaatttagtttattttgccaaggttaaagacattaccgtgacacagcctcaggaggtcctggtgacctgtatccaaggtggtcaggctacagcttggttttatacattataggaagatataagacatcaatcagtacttgtaagatgtacactggtttggtccagaaaggcaggagaactcaaagtggtttggggttggaggtgcaggggagcttccaagtcatgggtgcattcaaagattttctgattggcattggttgaatgagtttatctaaagatctagaatcaatagaagagagtgtcgggattagataaggggttgtggagaccaaggttcttattatgcagaagaagcctccaggtagcaggcttcagagacaattgattgtaaatgtttcttatcagacttaaaaaggtgccagacttagttcattctctcccagatcagtgaaaagatctggatagggaagggaattctctacagaatgtagattttcccaccacaagacagctgttcagggccctttcaaaatatgtcaaagaaatatattttagggtaaaatagtttaatttctttcagggcctgctgtctgtcatgtgatgctatactagagtcaggctggaatttggtgtcttattgctacaaaaagtctgcttcatcagtcttaaaatctgggttttaatgttaatgctggtcagttgtgcctgaattccaaagggaggagagtataaagaggcgtgtcccacctccccttcccaccatggcttgaactttggaatgcccttggctgagaggaggggtccattcagatggttggagggcttagaattttatttttagtttacatgggcttgagcatttaagcaccaatcacatacagggcactgtggtaggcgccagatactacctgagctagctcttacctacagcagaaaactcttggtccacaagtcaactcttagtgtttggttctttttgtgaacataaataaatatttggtgcttcagaaacaacccctttagtaaacacagcccatcccacatggccatcctggtcagacaggacatccacccaagcccgaaccctcacgtccctcctcagcctctccagctcccaccaactgctcaatgttatgcactgtgggctgagcagacagggaagccagagatggccccactagggcctgcagtgcagaggcaggtggtgaggggcggagcaggtgttcccaagaggtcccaagagcctttccagtgagttcataacccagaaattcaagtggtatcttttgacttggcacagatttccaatttcaatcagcctgctctgccagcctccccatctgtccttcaccctgttacccacctgctcactctgaggctccccagcctctctggcctctcaaccctcagcatggactctccctggaaagtgacactagcccctcacccactccagagatgatcctgagcttcccgaaaattgcccccagctttggatctctgatcatcacactgattcagtcattcagcaagcatttgttgatgctctgtccaacacctacctaaggatacagaggaccctagcctctggatgctcacaacctggcagggcggggggattcctaaacaattcttcacagcatactcgggaagagctatgccaagccacacaagggacccccccccaaccccaccatccatctgcctcgccctaaatccactgctgcctctgcactgccaaaaatctctcccaggggatggttctcaatagtggaaagggcatctcgctgctctgtcaacgttgggatattttagtgtcttggagattagtgagtaaagctgatcatgatacctcagagtctgcatttaaatcttgctctaaatcggtgcatcctttttatgtgtagtttttctattactgttatgcctcactgttttaacagaggtttctatttatatggggggaggagcattacttgggagagagtgcatgctgcaggcatactatgttaaacctacagagagctgaactgtccagacgccgttgccaaagccccactgtctccacagcggcttcttcctagaagctaagctagagaaagttaactagatttgttatagtgggacttctcggagcctttttgatgtgttaaccagaaaatagtatagttttcgcaattttattatctggaaatcccttttttatcctggcacacctactgctaactctatcaatagtctgttgaacactagtttgggaaatgcagcaaaaatcccaagtcattcagggaaacacaacacatcctgaatgtgtgtgaccgtcactttgctgttacgactaaactgattgtgtctgattttctttgtgtattcaaggggcaaaggaattgctagtaataaaacgtattcctttcttatcacgctggatgtggatatcggcgagctgatcatgatcaagttcaagtgggaaaacagtgcagtgtgggccaatgtctgggacacggtccagaccatcatcccatggagcacagggccgcgccactcaggcctcgttctgaagacgatcagagtcaaagcaggagaaacccagcaaaggtgactgctgattcaatctcctattaacgtccattaagcacccacttgtgccaggcagtctcacaatttaatactcacattcatcatgtgaggtgagtattattaggccctagtgatggagtacagaagctcagaaaggtgaagtgactatcccaactttacacagccacaggtgccaggacaaggattcacacccaggtctttgaatacacaccagggtctccccactgcccgtggctaacgctgcctgcctctggagcctgaggaccatcacacgaaccccaggcttctcaagcttcggtgcaggtgaatcaccaggtgatgtcagtgctgccagtccaaggtgctgtggtagatgcaacatcttttttttattgtttgcccaaagaaaatcttttaacagatcacaatatcaaaaatctcgatgtatctctctctctctctctctctctgtggtcgtatctttcaaatgtcagttgccaaagaatcactgggaagccaaacacatttgcagaattcctggacctacccgcagattcatggtgggcttgggaaggggtccaggatgcctgttgttgaatcagagccctaagaaactccaatgtagaacaatgggtcacccgaaagaaatgctgctgtgtttgatttaacatctcccgtggccccaaagtctccagggatacgaaggagctggggtggggagcaaagaaaaggaaggacagggctgggcgtggtggctcacacctgtaatcccagcactttgggaggccttggcgggcggatcacctgaggtcaggagttcaagaccagcctggccgacatggtgaaatcccgtctctacaaaaatacaaaaattagctgggcgtggtggcgcacaatggtagtcccagctactcggggggctgaggcagaagaatcagttgaacccaggaggtggaggttgcagttagccgagatcgcggcattgcactccagcctggatgacagagagagagactccgtctcaaaaaataataataataatagaaaagaaaaggaaggatgctccagcgctcctgaagtcctcccagcaaaagaaagggctgcccagcacaccctgccatagcaaccgtgcttttcggtcatccgtggttctccacggcagaattccaggcatccccgggccctctcctctctcttcccttctctattcaggaaacggtctctcctctcccaccttcacttgtcgcgaaggccatgtttctctttggcttgtcctcatagaggtggaaaacagcaggtttctaacctctgaactaccctcttccgtcaccccctgggtttcactgccagatgccggcctgtggcagatgtatgcccctgagtccctttatacaaccgcactctgagattttatctccctgggatgcaaaatcctggcaattactgagagcatactctgccgtctgccaacagatctcccaacaggatcaatctgggaagattaaactgctcgctcctcttctgcactgagctcttctcacatgactctttggcccattggagcagcgctgcttctgagctctgaccctgcccagatgctcttagctggagagtaattcctcagtttaggtggctgctcaccctgacaatcccatgtggcttgacctgaaagggtggctaaagcacaacactaccttctccactggggcacacccatgtggtacggaagaagaaatgaggagagggaggggcctggcccttccagggctcccacacggggtgagcattgaagcaggtgctccatggcattggtctcacgtgatctttgctgtgatcctgaaatgggtagtattggctgtgcagattaggaacctgaggctcacagaagttacacagcttgaggacattgctcaaaattgtcccagtccagatttgactgggatttgtctgatttattacccatttgttgggggtgtgctgtttccaacagttccttcttccattagttttggttccattaatctgaattttctcagtctaatctagcagagttgcagtgctggacattaacatatgtatgcatcaaacccatccttagggctgtaggaggtactgtatgcgggttatgggagctataattttttttaggttgtaaataccgagtgcctcctctgtgtctacacagtgccaggtactatcggagaatacaaaaaaaaaaaaaaaaatctgagttgtggctcttggtagaaataaactccaggacctaaccaggaaaataagatgaattaattttactaacggtgtggtccatcccagggcttctcaaactttcacgagcatacaaagcatctgggaatcatgttaaaatgcaggttctgctttagtgagtctggggtgagcctgatgttctgcatttctaacaagcgaccaggcaatgctgttgctgcaggtcaacagaccacactttgaatagtgagagtccagcattacacaacccgggaagtgtaagaagaaagttcagacccgtacacttggggctgtgctgcaacatgctttgaggaggccaagaggtgttcccagggagacaggcagccggcaaagcaatatggctggcaggatcggatggactccagtgtcagccagtcctaaactcacattttaacttcactgctcacaatctgtgtagcctcaaacaataacttcactcttcaagactcagttgtttagtgataaacgagaatcataatatacatttcatgagactaataacaactactaattttttagtgccttttgcagaccaaaatcccagaatcgggatgggaacattaaactctacactcgtaatatcgctgctaagtgcaagtgggaaaagaagtaggttaagtaacttgctctgtgcctccatggaagaaattcagtggaaggtggttttcttcccccaaaggcacaaaatcagggatgagtagggttgtggagatggtgaagagaaagagaagaaattattggccctacagcaagaccaagtataataactggctaaacctaatgagaagacattcaatttccacagtactcatataaatgcaaaataaaatcaaagaaattatcactggttactcattagtttggcaggaattcaaaaacggacaacacccagagttagtaaagatgtggagaaacgggcattctcatacactattagtgagagtgtaaattacaggcattttgtaggggaatttcattcaacaactaacgtttagtgagtacctaccttatgccagacaatgttgtggacaccagatgtataatagtgaacagacaggctgggctaaaatacaaaaaaattagccgggcgtggtggcgcatgcctataatcccagctactcgggaggctgaggcaaaggaatcgcttgaacccaggaggcagaggttgcggtgagctgagatcacaccactgcactccagcctggcgacagagcgagactccgtctcaaaaaaaataaaaaatatatatatatatatgtatatatgtgtgtgtgtgtatatatatatgtatatgtgtgtgtgtgtgtgtatatatatatacatatatatatacatatatatgtatatgtgtatatatatatatatgtatatgtatatatatatatgtatatgtatatatatatatagtgaacagacagagtccttggtctcatgaagtttacattatcacatgtttaaatatgcatactactttgagccagaattttatttctaggtgtcttatcatctttctggagtatctgtgcacataatcaaaatatgcacaaaggctatatatgaatacatatgtacagaatgtttgttgcagtactattaaataaatactatacataataagtaaatagtaaactcttgagaaaatcttaatgtctaacagtaggaaaatgcattgtggttcatccttaaaatggaatgtcatgaagcaggtgaaaaatagtgaaatagacttctacctactgatatgagcaagtctctgagtaccattttctggtaacaggaaaaattgcaggaaaacatacacaacatgatgccactcaatatatgtaaatgcataggaacaagtctggaaatgcataaaccaaatcataaatgtagagaggggagtgatgttagtgaaggccgatggtgaagaggttctttattatttggcttcatttattcctttaccatttgaattttcaagaatgcattcacaaattactcatagcatttttaaattttaaaagtccagtttagtgaagacttgaccttagacgcaattcaaagaaaaaagaagtaataaactagaatttttctacagatatatctatagatatatatttaacagatgcaattggaaataataacacaaaatggtatctcgggccaagccaacagccaaagcagtctagttgttgtttccaaaacacatagtagtgtcctctttagtggacaatgtcagctccaatatgaggaaggggcagggcttacagggctgagccaggcagaaaccgatgacactggcctcatgccaaggacctttaagaacagcatttacaaactggagcttgtccaaggagggaaggctgaggaggtaagcttgccttaggaggaagggttgaggaactagaaatgtttgccctgaagtcaaggcaaatgtggtcaaggaaacaatagtcaccctcaaatcttcgatgagctgtcataggggagaacaaaggagggctgccccaggactttccttcttacagaactgaaatcaccccagtgtagaactccaggaagtcttacactgatggctttcactgacttgggttttgtttttctctgaagttacaaaaatatttgcgaacataagaatctaacttaaaaaaagacatcacatgccttacacaaattgagtgaaatttgataaagaatgaaacacttcaataagctccacctaaaacttaatgctgtgtttgcttcctgttttctattcagaatgacattttgttcagaaaacacagatgacctactacttcgcccaacccaggaaaaaatcttcgtgaaatgtgaaataaagtctaaaacatcaaagcgaaagatcagatgagatttaatgaagacccagtgtaaagaataaatgaatcttactcctta

SEQ ID NO: 2 - DNA, 1604 bp, Homo sapiens
ggtctctttggcttcagaaattaccaagaaagcctggaccccgggtgaaacggagaaatggacacaagtcccctgtgtttctccattctgttggttttatgcatctttatccaatcaagtgcccttggacaaagcctgaaaccagagccatttggaagaagagctcaagctgttgaaacaaacaaaacgctgcatgagatgaagaccagattcctgctctttggagaaaccaatcagggctgtcagattcgaatcaatcatccggacacgttacaggagtgcggcttcaactcctccctgcctctggtgatgataatccacgggtggtcggtggacggcgtgctagaaaactggatctggcagatggtggccgcgctgaagtctcagccggcccagccagtgaacgtggggctggtggactggatcaccctggcccacgaccactacaccatcgccgtccgcaacacccgccttgtgggcaaggaggtcgcggctcttctccggtggctggaggaatctgtgcaactctctcgaagccatgttcacctaattgggtacagcctgggtgcacacgtgtcaggatttgccggcagttccatcggtggaacgcacaagattgggagaatcacagggctggatgccgcgggacctttgtttgagggaagtgcccccagcaatcgtctttctccagatgatgccaattttgtggatgccattcatacctttacccgggagcacatgggcctgagcgtgggcatcaaacagcccataggacactatgacttctatcccaacgggggctccttccagcctggctgccacttcctagagctctacagacatattgcccagcacggcttcaatgccatcacccagaccataaaatgctcccacgagcgatcggtgcaccttttcatcgactccttgctgcacgccggcacgcagagcatggcctacccgtgtggtgacatgaacagcttcagccagggcctgtgcctgagctgcaagaagggccgctgcaacacgctgggctaccacgtccgccaggagccgcggagcaagagcaagaggctcttcctcgtaacgcgagcccagtcccccttcaaagtttatcattaccagttcaagatccagttcatcaaccaaactgagacaccaatacaaacaacttttaccatgtcactactcggaacaaaagagaaaatgcagaaaattcccatcactctgggcaaaggaattgctagtaataaaacgtattcctttcttatcacgctggatgtggatatcggcgagctgatcatgatcaagttcaagtgggaaaacagtgcagtgtgggccaatgtctgggacacggtccagaccatcatcccatggagcacagggccgcgccactcaggcctcgttctgaagacgatcagagtcaaagcaggagaaacccagcaaagaatgacattttgttcagaaaacacagatgacctactacttcgcccaacccaggaaaaaatcttcgtgaaatgtgaaataaagtctaaaacatcaaagcgaaagatcagatgagatttaatgaagacccagtgtaaagaataaatgaatcttactcctta

SEQ ID NO: 3 - DNA, 3199 bp, Homo sapiens
cctccctggtcagcttcaagaaaacacaacagagcttgtgcattaaaaccaccactggaaggtcagacccggtgatggggcaggctggaagagcttaggtagaggtgaaagcagacaacaccctgaaaggggacaggtacccactcctccttgtcattttccaggagagccttatagaatttgagttggcatgagatgatgttagaggaatttggggtggtgcaagtaaaggaactacaagagctagaaacaatcttgaaggcttcttcccatgttgaaagtgttcttaccacgtagaaagtggtaagaaagtgtactctgcagaaatttggggggcgggtgggggcacacggttggtatttcagaggcacttggattcactggtgtcttttgatgagtttgtgtcccgaataaaatggggagtggcggtggttactggcagtacccagaggagaggagatgagggtgggcacaggggagaggagcccgagatgggtagccagctggtcttacgtgccagctttataggaaaaaaagtggaactttcagattctgcttaactgtgcaggagccagccaagtatccaaggcagaagtccttgctcagaaattgtatatctgatgggcacggtgactcacgcctgtaatcccagcactttgggaggccgaggtgggtggatcacaaggtcaggagttagagaccagcctgaccaacatggtaaaaccccgtctctactaaaaatacaaaaattagccaggcatggtggtgggcacctgtagtcccacctactctgaaggctgaagcactagaatcgcttgaacctgggaggtggaagttgcagtgaaccgagatcgcgccactgcactccagcctgggcgacagagcaagattccatctcaaaaaaaaaaaagaaaaagaaaaagaaattgtatatctgacgggatggtccgcaagcttcggggcttgggaggaaggtacagtcataggacagaacacagctttagactgggtcaagatcgtccaagtctggtggtggcttcagaaggccctttgaatgaggctcgtccaggcaataattaccatgttatctaattttgcccgcttaacgtttccacttggatgtctaagagccatctcaaagccccacgtggccaaggctgaatctctaatcttcccccaacagacctgcttcactcacagcttccccttctcagctgacagcaaccccattaatctgcttgttcaagccaaaaaacctggaagtcatccttaactcctctctttctcccacatctcacaactcagcagtcagaaaatcctgtgggctctaccttcaaaatatatccaccacccatccaccactccgcagttgccctttaccatgctgggccacgccaccatcacctctcactgggccactgtaatagccttctaacagatctccccactgcaccctggctcacttgcaaaccagagtgatattttaaaactataatacaaatcatatcacccttctgctcagagtcgccccccatgtgtcaatgcataaaagtcaaagtccttacaatgtctccccattcattggcctccttgctattcctggagcacaccaggcatactcctacctcagggcctttgcacagactgcccgaagctctcctccccaagacgtccacaggggcaactccttcacttccttcaaacctttgctgaaagtttaacttcccaatgcaattcactctaaccactctgcttaaaatccctaccttctccccaaccccctgcttttcttgtagtggttcccattacattctaccagtctacctaatttttttttttttttttttgagacggagtctctctgtgtcgcccaggctggagtacaatggcgcgatctcagttcactgcaacctccgcctcccaggttcaagcgattctcctgcctcagcttcctgagtagctgggattacaggcactcacctccacgcccagctaacttttgtatttttagtacagatggggtttcaccatgttggtcaggctggtctcgaactcctgacctcgtgatccgccctcctcgcctcccaaagtgctgtgattacaggagtgagccaccgctcctggccagaaatctcttctttattatgtctactgtccgttatccaactccagaaggtaagaacctccactgatacataaggacttgtataccccacgtgcctgcaacagtgcttggcacctagtaggcataccaaaatatataaatgttgaacaaatgaagaaagttaaagtaaaactagaggtccaaaaatatcacaaaagccatctatggtcgccttttccctacctgattttgctgagtggccttacttttcagtcctctacacagctggaacattaatgaacacagagggggaagaagtgtgtttactctaggatcacctctcaatgggtcacttggcaagggcatctttgcttcttcgtcagctccttttgacacgggggtgaagggttttctgcaccacactttgaccacaagcatcaccaatttcactgaacccaacagaaatttggaccctctgggggctctctgcgtggcagggcccttttctttttctttgggcttaggctgcaatttgaaacaccactttcctgagccagcatcccccttgcagcgctgtcacagggaggcttaggcagccacgtggaagccacctaccccgacctttggcagaatttccaaacacaacacagtagctttaagttgattaatttggaactctgaccttggccccaaaaggtaagaatacataacaaggtattttattctcaaaatgtgtcaggataagaagcacttctgtaaatcgacctttttaaaatagatataattagatttgcagttgggggcagtaaagaaagggtctgaacagtggataacatgttgagaggttaattattaatgggcagtcttccctaacaaagtatctaataggcattgtggtctctttggcttcagaaattaccaagaaagcctggaccccgggtgaaacggagaaatggacacaagtcccctgtgtttctccattctgttggttttatgcatctttatccaatcaagtgcccttggacaaagcctgaaaccaggtaagagcctgacttttctccagagatgggcatgaacttttctttttaaaacgtg

SEQ ID NO: 4 - DNA, 61 bp, Artificial Sequence
  chemically synthesized nucleic acid probe
gaatttccaaacacaacacagtagctttaarttgattaatttggaactctgaccttggccc

SEQ ID NO: 5 - DNA, 61 bp, Artificial Sequence
  chemically synthesized nucleic acid probe
tctttgcttcttcgtcagctccttttgacaygggggtgaagggttttctgcaccacacttt

SEQ ID NO: 6 - DNA, 68 bp, Artificial Sequence
  chemically synthesized nucleic acid probe
aaatgaagaaagttaaagtaaaactagaggyccaaaaatatcacaaaagccatctatggtcgcctttt

SEQ ID NO: 7 - DNA, 79 bp, Artificial Sequence
  chemically synthesized nucleic acid probe
ctgcaacagtgcttggcacctagtaggcatyccaaaatatataaatgttgaacaaatgaagaaagttaaagtaaaacta

SEQ ID NO: 8 - DNA, 20 bp, Artificial Sequence
  chemically synthesized primer
gctgtcacagggaggcttag

SEQ ID NO: 9 - DNA, 20 bp, Artificial Sequence
  chemically synthesized primer
ttctttactgcccccaactg

SEQ ID NO: 10 - DNA, 21 bp, Artificial Sequence
  chemically synthesized probe
aattaatcaacttaaagctac

SEQ ID NO: 11 - DNA, 21 bp, Artificial Sequence
  chemically synthesized probe
aattaatcaatttaaagctac

SEQ ID NO: 12 - DNA, 20 bp, Artificial Sequence
  chemically synthesized primer
ccatctatggtcgccttttc

SEQ ID NO: 13 - DNA, 20 bp, Artificial Sequence
  chemically synthesized primer
attggtgatgcttgtggtca

SEQ ID NO: 14 - DNA, 20 bp, Artificial Sequence
  chemically synthesized probe
cttttgacacgggggtgaag

SEQ ID NO: 15 - DNA, 20 bp, Artificial Sequence
  chemically synthesized probe
cttttgacatgggggtgaag

(19) United States
(12) Patent Application Publication
(10) Pub. No.: US 2010/0087462 A1
(43) Pub. Date: Apr. 8, 2010

(54) METHODS FOR PREDICTING AND DETECTING INTOLERANCE TO AN HYPOLIPIDEMIC AGENT

(75) Inventors: Daniel GAUDET, Chicoutimi (CA); Marta SANTURE, Chicoutimi (CA); Diane BRISSON, Chicoutimi (CA); Gerald TREMBLAY, Chicoutimi (CA)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: BOREAL PHARMA RECHERCHE CLINIQUE, Chicoutimi (CA)

(21) Appl. No.: 12/245,088

(22) Filed: Oct. 3, 2008

(51) Int. Cl. A61K 31/505; G01N 33/92; C12Q 1/68; A61P 9/00; C07H 21/00; G01N 33/98

(52) U.S. Cl. 514/275; 436/71; 436/131; 435/6; 536/24.31

(57) ABSTRACT

The invention describes methods and tools for the identification of drug intolerance into subjects receiving an hypolipidemic agent. The invention further describes methods and tools for the prediction of drug intolerance in subjects in need of hypolipidemic agents. Some aspects of the invention are based on the assessment of the subject plasma glycerol concentrations. Some aspects of the invention are based on the assessment of the subject HDL-triglycerides concentration. Some other aspects are based on the identification of hepatic lipase gene variants in a subject genome.

### FIELD OF THE INVENTION

The invention relates to the field of hyperlipidemic diseases and disorders. More particularly, it relates to the identification of drug intolerance into subjects receiving an hypolipidemic agent, and to the prediction of such intolerance in subjects in need of hypolipidemic agents.

### BACKGROUND OF THE INVENTION

Hypolipidemic agents, or antihyperlipidemic agents, are a diverse group of pharmaceuticals that are used in the treatment of hyperlipidemias. However, many side effects are related to the treatment with these drugs. For example, there are many known side effects associated with satins, the most widely prescribed class of lipid-lowering drugs, Statins are used to lower cholesterol levels in people with or at risk of cardiovascular disease and side effects include headaches, an elevation in liver enzymes, muscle pain, myopathies, and gastrointestinal problems. Therefore, it would be beneficial to the physician and its patient to have access to tools and methods for predicting a patient susceptibility to the side effects associated with hypolipidemic drugs treatment. Also, it would be beneficial to the physician to have a biological marker providing means for detecting an intolerance to hypolipidemic drugs before the side effects occurs.

Glycerol (Propane-1,2,3-triol) is a chemical compound also commonly called glycerin or glycerine. Glycerol is involved in the metabolic roads of glucose, proteins, pyruvate, triacylglycerols and other glycerolipids. When the body uses stored fat as a source of energy, glycerol and fatty acids are released into the bloodstream. Glycerol involvement in several metabolic pathways stresses out the important role it plays in homeostasis among healthy individuals as well as individuals whose glucidic or lipidic metabolism is abnormal. Glycerol plasma concentrations normally do not exceed 0.08 mmol/L. In subjects, glycerol plasma concentration are currently not monitored but they could possibly be used to predict glucose intolerance and diabetes (see U.S. Pat. No. 6,743,579 which describes the use of glycerol as a predictor of glucose tolerance). Preliminary results about a relationship of plasma glycerol levels and muscular markers of statin myotoxicity were presented by the inventors during a poster presentation at the XVI International Symposium on drugs affecting lipid metabolism. New York, N.Y. (USA), Oct. 4-7, 2007.

Lipoproteins are high molecular weight globular particles. Lipoprotein particles include very low density lipoproteins (VLDL), low density lipoproteins (LDL) and high density lipoproteins (HDL). HDL particles can contain variable quantities of triglycerides and cholesterol. In the literature, the term “HDL-triglycerides” identifies the triglyceride content of the HDL particles whereas the term “HDL-cholesterol” refers to the cholesterol content carried by the HDL particles. The same principle applies for the other lipoproteins, such as LDL-cholesterol or LDL-triglycerides. In subjects, HDL-triglycerides plasma concentrations are very often monitored in research for the evaluation of metabolic syndrome and cardiovascular disease risk. However, no one has ever suggested a correlation between the plasma composition in HDLs on the appearance of side effects following treatment with hypolipidemic agents, such as statins.

Hepatic lipase (HL) is a key enzyme involved in lipoprotein metabolism. HL catalyzes the hydrolysis of triglycerides, diglycerides and phospholipids in lipoproteins. Specifically, HL promotes the conversion of larger and less dense HDL₂ particles into smaller and denser HDL₃ particles. An HL deficiency is characterized by HDL and LDL particles enriched in triglycerides. Recently, four common polymorphisms in the promoter of the HL gene have been described. These polymorphisms are in complete linkage disequilibrium, and have been identified as: −514C>T (also called −480C>T); −250G>A; −710T>C; and −763A>G. Together, these four polymorphisms are designated as “−514CIT alleles” (Zambon A., et al. Curr Opin Lipidol 14: 179-189).

It has been shown that a bi-allele distribution is responsible for approximately 30% of the variation of the HDL cholesterol rate in the general population (Cohen et al., Curr. Opin. Lipidol. 1999. 10(3):259-67). The presence of the C allele has been significantly associated with a high HL enzyme activity and lower levels of HDL cholesterol (Jansen et al. Arterioscler. Thromb. Vasc. Biol. 1999; 19(2):303-8; Zambon et al., Arterioscler. Thromb. Vasc. Biol. 1998; 18(11):1723-9). However, the protective effect of the T allele is eliminated in the presence of abdominal obesity and in addition is significantly associated with higher levels of HDL triglycerides (St-Pierre et al. Mol. Genet. Metab. 2003 78(1):31-6). It has been demonstrated that the −514 C/T alleles of the HL gene influence the clinical response to hypolipidemic agents like statins (Lahoz, Atherosclerosis 2005. 182(1):129-34; Cenarro et al. Am. Heart J. 2005; 150(6):1154-62). However, the influence of −514C/T alleles on tolerance to hypolipidemic agents, such as statins, had not been demonstrated before the present invention.

Thus, there is a need for methods and biomarkers that may be helpful in detecting and predicting a subject intolerance to hypolipidemic agents. There is also a need for methods to detect such intolerance to identify the risks of developing side effects associated with treatment with statins. Yet there is a need for improved method of treatment wherein side effects may be prevented, or at least predicted and monitored.

Features of the invention will be apparent from review of the disclosure, drawings and description of the invention below.

### BRIEF SUMMARY OF THE INVENTION

The invention relates to the identification of drug intolerance into subjects receiving an hypolipidemic agent, and to the prediction of such intolerance in subjects in need of hypolipidemic agents.

The invention provides a method for detecting an intolerance in a subject receiving an hypolipidemic agent, comprising assessing a plasma sample from said subject for measuring therein the concentration of a lipidic biomarker and correlating said concentration with a threshold value, wherein an abnormal concentration of said lipidic biomarker is indicative of an intolerance to the hypolipidemic agent.

The invention further provides a method for detecting muscular intolerance to an hypolipidemic agent in a subject receiving said hypolipidemic agent, comprising assessing said subject resistance to glycerol.

The invention also pertains, at least in part, to a method for detecting intolerance in a subject receiving an hypolipidemic agent, comprising assessing a plasma sample from said subject for measuring therein glycerol concentrations, wherein a concentration of glycerol greater than about 0.2 mmol/L is indicative of an intolerance to the hypolipidemic agent.

In yet another embodiment, the invention pertains, at least in part, to a method for monitoring, treating, improving, or alleviating hyperlipidemia in a subject, the method comprising the step of detecting intolerance to an hypolipidemic agent in a subject receiving said hypolipidemic agent; and/or predicting an intolerance to an hypolipidemic agent prior to administration of said hypolipidemic agent to a subject.

The present invention further relates to a method for selecting a desirable dose of an hypolipidemic agent administered to a subject, the method comprising monitoring glycerol plasma concentrations of said subject for detecting an intolerance to said hypolipidemic agent; and adjusting said dose for reducing or eliminating side effects while maximizing a desired plasma lipid reduction.

The invention also pertains, at least in part, to improving a method for reducing risks of cardiovascular disease in a subject which comprises administering an hypolipidemic agent to the subject, the improvement comprising monitoring glycerol plasma concentrations of said subject for detecting an intolerance to said hypolipidemic agent.

The invention also provides a method of detecting intolerance of a subject to an hypolipidemic agent, comprising assessing a biological sample from said subject for identifying a hepatic lipase gene variant, wherein presence of said hepatic lipase gene variant is predictive of a reduced risk of intolerance to hypolipidemic agents.

The invention further provides a method of detecting intolerance of a subject to an hypolipidemic agent, comprising assaying DNA from said subject for the presence or absence of a hepatic lipase gene variant selected from the group consisting of: an adenine at position 2764 of SEQ ID NO:3 (−250 G/A); (ii) a thymine at position 2500 of SEQ ID NO:3; (iii) a cytosine at position 2304 of SEQ ID NO:3 (−710); and (iv) a guanine at position 2764 of SEQ ID NO:3; wherein presence of one or more of said gene hepatic lipase gene variant is indicative of a reduced risk of intolerance to the hypolipidemic agent.

A related aspect of the invention concerns a method of predicting intolerance of a subject to an hypolipidemic agent, comprising assessing a biological sample from said subject for determining nucleotides present in the promoter region of said hepatic lipase gene and comparing said nucleotides with a reference promoter nucleotides as set forth in SEQ ID NO: 3, wherein identification of a hepatic lipase gene variant at nucleotide position 2764, 2500, 2304 and/or 2764 of SEQ ID NO: 3 is indicative of a reduced risk of intolerance to the hypolipidemic agent.

The invention also provides a method of for predicting intolerance of a subject to an hypolipidemic agent, comprising:
a. obtaining from said subject a biological sample having DNA;
b. sequencing regions of said DNA encoding hepatic lipase; and
c. detecting in the sequence obtained at (b) a gene variant that is predictive of intolerance to the hypolipidemic agent.

The invention also pertains, at least in part, to a nucleic acid probe for predicting intolerance of a subject to an hypolipidemic agent, said probe comprising at least 20 contiguous nucleotides of any one of SEQ ID NO: 4; SEQ ID NO: 5; SEQ ID NO:6; and SEQ ID NO: 7, and wherein said probe comprises at least 10 contiguous nucleotides extending on each side of nucleotide at position 31 of SEQ ID NO: 4, SEQ ID NO: 5; SEQ ID NO: 6; or SEQ ID NO: 7.

The invention also provides a solid support comprising a probe as defined herein and a kit for detecting and/or predicting intolerance of a subject to an hypolipidemic agent, the kit comprising a user manual or instructions and (i) a probe or primer specifically hybridizing to a nucleotide sequence comprising a hepatic lipase gene variant.

Additional aspects, advantages and features of the present invention will become more fully understood from the detailed description given herein and from the accompanying drawings, which are exemplary and should not be interpreted as limiting the scope of the invention.

### BRIEF DESCRIPTION OF THE DRAWINGS

In order that the invention may be readily understood, embodiments of the invention are illustrated by way of examples in the accompanying drawings.

FIG. 1 is a graph showing a relationship between the glycerol level (fastening) of subjects who have not yet been treated with statins and the values of creatin phosphokinase (CK) in the same individuals once they have been treated with statins. Statistical significance: (1) p<0.05; (2) p=0.05-0.08; (3) p=0.08-0.1; (4) p=0.1-0.2. Age, gender, statin dose in quartiles and combination with other drugs were included in ANOVA as covariates.

FIG. 2 is a graph showing the relative risks of presenting statin side effects associated with pre-treatment plasma glycerol concentrations >0.2 mmol/L. Age, gender, statin dose in quartiles and combination with other drugs were included in ANOVA as covariates.

FIG. 3 is a graph showing the relative risk of developing muscular and non-muscular side effects to statins (Odds ratio±SE) in relation to plasma concentrations of HDL-triglycerides (mmol/L) before treatment. Adjusted for age, gender, statin dose in quartiles and combination with other drugs.

FIG. 4 is a graph showing a relationship between plasma HDL-triglycerides concentration before treatment with statins and CK plasma levels after treatment with statins. Adjusted for age, gender, and waist size.

FIG. 5 is a graph showing a relationship between HDL-triglycerides concentration before treatment with statins and CK plasma levels after treatment with statins among carriers of alleles −514 CC, −514 CT and −514 TT.

FIG. 6 shows sequences around polymorphic sites −250, −480(−514), −710 and −763 of the hepatic lipase gene promoter region, including wild/alternative nucleotides.

Further details of the invention and its advantages will be apparent from the detailed description included below.

### DETAILED DESCRIPTION

### I. Definitions

As used herein and in the appended claims, the singular forms “a”, “an”, and “the” include plural referents unless the context clearly indicates otherwise. Thus, for example, reference to “a variant” includes one or more of such variants and reference to “the method” includes reference to equivalent steps and methods known to those of ordinary skill in the art that could be modified or substituted for the methods described herein.

The term “gene” refers to a nucleic acid comprising an open reading frame encoding a polypeptide, including both exon and (optionally) intron sequences. The nucleic acid may also optionally include non-coding sequences such as promoter or enhancer sequences. The term “intron” refers to a DNA sequence present in a given gene that is not translated into protein and is generally found between exons.

“Hepatic lipase” or “HL” as used herein refers to the enzyme which is described herein at Example 2. The hepatic lipase protein is encoded by the hepatic lipase gene that is found on chromosome 15 (GenBank Acc. No. NC_{—}000015 REGION: 56511467.56648364). The gene comprises seven (7) introns and nine (9) exons. The latest public version of the entire sequence of the hepatic lipase gene is provided in SEQ ID NO: 1. The coding sequence of the hepatic lipase gene is as set forth in SEQ ID NO: 2. The sequence of the promoter region of the hepatic lipase gene is as set forth in SEQ ID NO: 3. A “hepatic lipase gene variant” refers to any known or unknown polymorphism that may be present in the hepatic lipase gene, and more particularly polymorphism which have an effect on the activity of the hepatic lipase enzyme. In preferred embodiment, the term “hepatic lipase gene variant” refers to polymorphisms that are present in the promoter region of the HL gene.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction.

The terms “percent similarity”, “percent identity” and “percent homology” when referring to a particular sequence are used as set forth in the University of Wisconsin GCG software program.

The term “oligonucleotide” as used herein refers to sequences, primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as appropriate temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

With respect to single-stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA molecule of the invention, to the substantial exclusion of hybridization of the oligonucleotide with single-stranded nucleic acids of non-complementary sequence. Appropriate conditions enabling specific hybridization of single-stranded nucleic acid molecules of varying complementarity are well known in the art. For instance, one common formula for calculating the stringency conditions required to achieve hybridization between nucleic acid molecules of a specified sequence homology is set forth below (Sambrook et al., 1989, Molecular Cloning: A Laboratory Manual, Cold Spring Harbor Laboratory Press):

Tₘ=81.5° C.+16.6 Log [Na+]+0.41(% G+C)−0.63(% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5 with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. With regard to the nucleic acids of the current invention, a moderate stringency hybridization is defined as hybridization in 6×SSC, 5×Denhardt's solution, 0.5% SDS and 100 μg/ml denatured salmon sperm DNA at 42° C. and washed in 2×SSC and 0.5% SDS at 55° C. for 15 minutes. A high stringency hybridization is defined as hybridization in 6×SSC, 5×Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5×Denhardt's solution, 0.5% SDS and 100 μg/ml denatured salmon sperm DNA at 42° C., and washed in 0.1×SSC and 0.5% SDS at 65° C. for 15 minutes.

As used herein, the term “solid support” refers to any solid or stationary material to which reagents such as antibodies, antigens, and other test components can be attached. Examples of solid supports include, without limitation, microtiter plates (or dish), microscope (e.g. glass) slides, coverslips, beads, cell culture flasks, chips (for example, silica-based, glass, or gold chip), membranes, particles (typically solid; for example, agarose, sepharose, polystyrene or magnetic beads), columns (or column materials), and test tubes. Typically, the solid supports are water insoluble.

As used herein, an “instruction material” or a “user manual” includes a publication, a recording, a diagram, or any other medium of expression which can be used to communicate the usefulness of the compounds or compositions of the invention for performing a method according to the invention.

As used herein, the term “biological sample” refers to a subset of the tissues of a biological organism, its cells or component parts (e.g. body fluids, including but not limited to blood, mucus, lymphatic fluid, synovial fluid, cerebrospinal fluid, saliva, amniotic fluid, amniotic cord blood, urine, vaginal fluid and semen).

As used herein, the term “subjects” generally refers to individuals and/or to human patients receiving an hypolipidemic agent or in need thereof.

The term “hyperlipidemia” as used herein, refers to the presence of raised or abnormal concentrations of lipids or lipoproteins in the blood of a subject. In some embodiments, the term hyperlipidemia refers more specifically to abnormally high glycerol concentrations. In some embodiments, the term hyperlipidemia refers more specifically to abnormally high HDL-triglycerides concentrations.

As used herein, the term “abnormal concentration” or “abnormally high concentration” when referring to a compound found in the plasma, generally refers to a level or concentration of the compound that is outside of generally recognize standards for a healthy individual. For instance, the following concentration can be used as standards for glycerol: very high dysglycerolemia (glycerol >0.2 mmol/l), high dysglycerolemia (glycerol 0.1-0.2 mmol/l), mildly high dysglycerolemia (glycerol 0.08-0.1 mmol/l), normal-lightly high glycerolemia (0.05-0.08 mmol/l) and normal glycerolemia (glycerol <0.05 mmol/l). For instance, the following concentrations can be used as standards HDL-triglycerides: the normal (about 0.1-0.15 mmol/l), high (about 0.15-0.5 mmol/l) and very high >0.5 mmol/l).

The term “intolerance” as used herein in the context of a treatment to a drug, it refers to an undesirable subject sensitivity to a drug, or to undesirable adverse reactions or side effects in a subject receiving a drug. For instance an intolerance to a statin may include side effects such as headaches, an elevation in liver enzymes, muscle pain, myopathies, and gastrointestinal problems.

The term “resistance to glycerol” or “glycerol resistance” as used herein refers to a state where glycerol becomes inefficient in its regular biological functions (e.g. protection of cells against statins, homeostatic effects) thereby resulting in a compensating raise of plasma glycerol concentrations.

### II. Use of Lipidic Biomarkers for the Detection of Intolerance to Hypolipidemic Agents

In some aspects, the present invention relates to lipidic biomarkers and methods for detecting intolerance into subjects receiving an hypolipidemic agent.

In one embodiment, the method for detecting intolerance comprises assessing a plasma sample from the subject for measuring therein the concentration of a lipidic biomarker and correlating that concentration with a threshold value. An abnormal concentration of the lipidic biomarker indicates intolerance to the hypolipidemic agent.

As used herein, the term “hypolipidemic agent” includes but is not limited to statins, fibrates (e.g. fenofibrate, bezafibrate), nicotinic acid, bile-acid sequestering agents, cholesterol absorption inhibitors (e.g. EZETIMIBE™), and any other suitable drugs that may be used to reduce to lower circulating plasma lipids, such as Peroxisome Proliferator-Activated Receptor (PPAR) agonists, inhibitors of the enzyme squalene synthase or as inhibitors of cholesterol ester transfer protein (CETP). In preferred embodiments, the hypolipidemic agent is a statin, or a combination of drugs comprising a statin. As used herein the term “statin” refers to inhibitors of the 3-hydroxy-3-methylglutaryl coenzyme A reductase generally used in clinical practice for the treatment of hypercholesterolemia. The term “statin” includes but is not limited to rosuvastatin, simvastatin, atorvastatin, pravastatin, fluvastatin and lovastatin.

Preferably, the lipid-lowering drug targeted by the present invention is a statin of a combination of drugs comprising statins. A person skilled in the art will readily understand that the term <<statin>> include the inhibitors of the 3-hydroxy-3-methylglutaryl coenzyme A reductase generally used in clinical practice for the treatment of hypercholestérolémie.

According to some particular aspects, the lipidic biomarker is glycerol. As described in Example 1, the inventors have established of a connection between glycerolemia and muscle intolerance to hypolipidemic agents (e.g. statins) among subjects with hypercholesterolemia.

According to the invention, glycerol has a protective effect against dehydration and cellular damage when the levels of CK rise up to a certain glycerol threshold value, above the threshold value, resistance to glycerol is observed. Accordingly, another aspect of the invention relates to methods for detecting muscular intolerance to an hypolipidemic agent in a subject receiving such hypolipidemic agent, the method comprising assessing the subject resistance to glycerol. According to different embodiments, the threshold value for glycerol is set at about 0.1 mmol/L, about 0.125 mmol/L, about 0.15 mmol/L, about 0.175 mmol/L, about 0.2 mmol/L or above. In preferred embodiments, glycerol threshold value is set at about 0.2 mmol/L. Glycerol plasma concentrations are thus a biomarker of intolerance to hypolipidemic agents such as statins.

Furthermore, the inventors observed a correlation between the glycerol plasma concentration in subjects who had not been treated with statins yet and the CK plasma concentrations the same subjects had been treated with a statin. Therefore, according to the present invention, determination and analysis of plasma glycerol levels among subjects who have not yet been treated with statins, provides means for screening and identifying subjects who would be at risk of developing myopathies and other side effects during a treatment with a lipid-lowering drug such as a statin.

Another related aspect concerns a method for detecting intolerance in a subject receiving an hypolipidemic agent such as a statin. The method comprises assessing a plasma sample from the subject for measuring therein glycerol concentrations and identifying an intolerance to the hypolipidemic agent when the concentration of glycerol is greater than about 0.1 mmol/L, greater than about 0.125 mmol/L, greater than about 0.15 mmol/L, greater than about 0.175 mmol/L, or greater than about 0.2 mmol/L (most preferred). In preferred embodiment the hypolipidemic agent is a statin and the intolerance to the statin is a muscular intolerance (e.g. myopathy).

Accordingly, another aspect of the invention concerns a method for detecting muscular intolerance to an hypolipidemic agent, preferably a statin, in a subject receiving the hypolipidemic agent, the method comprising assessing the subject resistance to glycerol. In a preferred embodiment, the subject resistance to glycerol is defined by a plasma glycerol concentration greater than about 0.2 mmol/L.

According to some particular aspects, the lipidic biomarker is HDL-triglycerides and an abnormal concentration of HDL-triglycerides is a concentration greater than the threshold concentration. As used herein, an abnormal concentration of HDL-triglycerides is a concentration greater than about 0.1 mmol/L, or greater than about 0.2 mmol/L, or greater than about 0.5 mmol/L. Preferably an abnormal concentration of HDL-triglycerides is defined as being greater than about 0.12 mmol/L.

Additional aspects of the invention comprise a method for detecting intolerance in a subject receiving an hypolipidemic agent such as a statin. The method comprises assessing a plasma sample from the subject for measuring therein HDL-triglycerides concentrations and identifying intolerance to the hypolipidemic agent when the concentration of glycerol is greater than about 0.2 mmol/L. In preferred embodiment the hypolipidemic agent is a statin and the intolerance to the statin is a muscular intolerance (e.g. myopathy).

Plasma concentrations of the lipidic biomarkers may be assessed using standards method and techniques, including, but not limited to, those provided herein in Examples 1 and 2. Examples of suitable methods include the measurement of glycerol by calorimetric analysis (RANDOX Laboratories Ltd, Crumlin, UK) using a Olympus AU400e™ instrument (Diagnostic Systems Group, Melville, N.Y., USA). Lipoproteins can be measured using for example the calculation of LDL-cholesterol levels using the Friedewald equation and well as ultracentrifugation methods, intended to separate different lipoproteins according to their density gradient and then measure contents of cholesterol and triglycerides, However, other methods could be used, including nuclear magnetic resonance techniques and direct measure of LDL, HDL, and lipoprotein debris rich in triglycerides. (Friday. Curr Atheroscler Rep. 2002. 4(5):359-62). Other techniques include automated methods used in clinical laboratories.

Those skilled in the art have enough experience in measuring plasma lipids concentration to use proper methods to obtain meaningful results that are in accordance with the present invention. For instance, although not specified everywhere herein, it is well known that plasma lipid concentration are measured in fastening subjects (about 12 h). Thus, it is implicit that the glycerol and HDL-concentrations referred to in this invention are concentrations measured in a fastening subject.

In some embodiments the methods referred to hereinbefore which relates to the assessment of glycerol or HDL-triglycerides concentrations are used in used in combination with methods, probes, primers and the like for the identification of hepatic lipase variants. Combination of genetic methods and more standard biological assays may improve sensitivity of the test and reduce possibilities of errors.

### III. Prediction of Intolerance to Hypolipidemic Agents

In some aspects, the present invention relates biomarkers and methods for predicting intolerance to hypolipidemic agents (e.g. statins) in subjects in need of such agents.

As shown herein after in the Exemplification section, the present inventors detected a relationship between different variants of the HL gene and certain side effects indicating statin intolerance. Knowledge of the variations in the hepatic lipase gene in individuals is thus useful for screening subjects who may develop myopathies and other side effects when taking hypolipidemic agents such as statins.

Subjects hepatic lipase (HL) gene variants were significantly associated with the subjects HDL-triglycerides composition. These results supports the rationale of using triglyceride content of HDL particles and/or certain gene variants frequently associated with HDL-triglycerides modulation as biomarkers for predicting potential side effects among individuals treated with hypolipidemic agents such as statins.

According to the invention, SEQ ID NO: 3 is a reference sequence for the hepatic lipase (HL) promoter. As can be seen, the following nucleotides are present: a guanine at position 2764 (−250 G/A); (ii) a cytosine at position 2500 (−514 C/T); (iii) a thymine at position 2304 (−710 T/C); and (iv) an adenine at position 2251 (−763 A/G). The inventors have found the presence of those reference nucleotides modify the relation between herein described markers of intolerance to hypolipidemic agents such as statins. Accordingly, presence of one or more hepatic lipase gene variant at any of those positions can be indicative of an enhanced or reduced risk of statin intolerance. Preferred examples or gene variant indicative of a reduced risk of statin intolerance include: (i) an adenine at position 2764 (−250 G/A); (ii) a thymine at position 2500 (−514 C/T); (iii) a cytosine at position 2304 (−710 T/C); and/or (iv) a guanine at position 2764 (−763 A/G).

Accordingly, an aspect of the invention concerns a method of detecting intolerance of a subject to an hypolipidemic agent, such as a statin, the method comprising assessing a biological sample from the subject for identifying a hepatic lipase gene variant, wherein presence of the hepatic lipase gene variant is predictive of a reduced risk intolerance to an hypolipidemic agent. In a preferred embodiment, the variant is present in a promoter region of the hepatic lipase gene. The variant is preferably selected from the group consisting of: an adenine at position 2764 of SEQ ID NO:3 (−250 G/A); (ii) a thymine at position 2500 of SEQ ID NO:3 (−514 C/T); (iii) a cytosine at position 2304 of SEQ ID NO:3 (−710 T/C); and/or (iv) a guanine at position 2764 of SEQ ID NO:3 (−763 A/G).

Another related aspect of the invention concerns a method of detecting intolerance of a subject to an hypolipidemic agent, preferably a statin. The method comprises assaying DNA from the subject for the presence or absence of a hepatic lipase gene variant selected from the group consisting of: an adenine at position 2764 of SEQ ID NO:3 (−250 G/A); (ii) a thymine at position 2500 of SEQ ID NO:3 (−514 C/T); (iii) a cytosine at position 2304 of SEQ ID NO:3 (−710 T/C); and/or (iv) a guanine at position 2764 of SEQ ID NO:3 (−763 A/G); wherein presence of one or more of the gene hepatic lipase gene variant is indicative of a reduced risk of intolerance to the hypolipidemic agent.

Yet, an additional related aspect of the invention concerns a method of predicting intolerance of a subject to an hypolipidemic agent, preferably a statin, comprising assessing a biological sample from the subject for determining nucleotides present in the promoter region of the hepatic lipase gene and comparing the nucleotides with the reference promoter sequence set forth in SEQ ID NO: 3. Identification of a hepatic lipase gene variant at nucleotide position 2764, 2500, 2304 and/or 2764 of SEQ ID NO: 3 is indicative of a reduced risk of intolerance to the hypolipidemic agent.

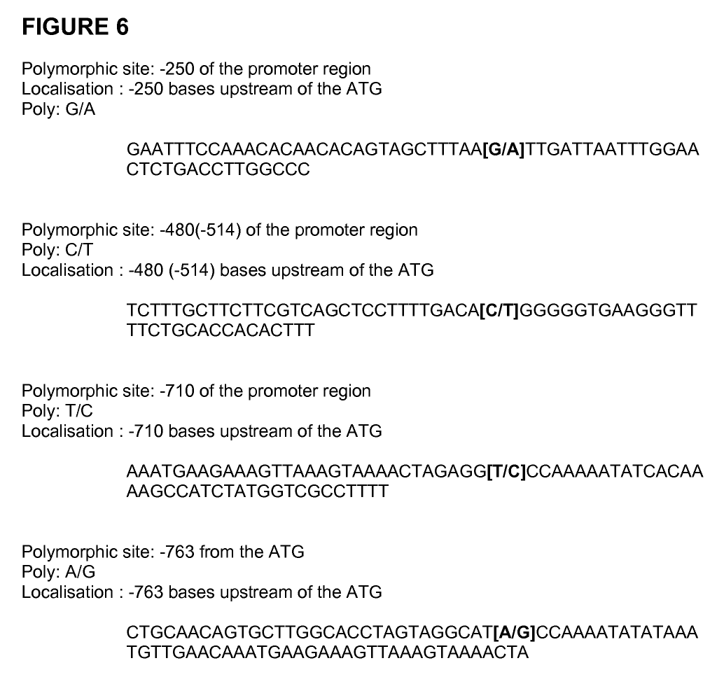

Another aspect of the invention concerns a method of for predicting intolerance of a subject to an hypolipidemic agent, comprising: (a) obtaining from the subject a biological sample having DNA; (b) sequencing and/or genotyping regions of the DNA encoding hepatic lipase for detecting a gene variant that is predictive of intolerance to the hypolipidemic agent. In preferred embodiments the gene variants are those shown in FIG. 6.

Although preferred embodiments of the invention focus on identification of hepatic gene variants in the promoter region of the hepatic lipase gene, the invention is not restricted to the sole promoter region. Similarly, the invention is not restricted to the four commonly known specific gene variants present in the promoter region referred to herein and commonly referred to as the “−514C/T alleles” shown in FIG. 6. Indeed, there are more than 439 gene variants currently known for the hepatic lipase gene (SEQ ID NO:1) and each of them could impact the hepatic lipase enzyme activity in regulating glycerol and/or HDL-triglycerides plasma concentrations according to the methods of the invention. It is conceivable that other hepatic lipase gene variants, in the promoter region, in the intron region, and/or the exon region, of the hepatic lipase gene may be useful according to the invention. A list of known hepatic lipase gene variants can be found on the NCBI single nucleotide polymorphism database at geneID:3990. Accordingly, the present invention encompasses all additional hepatic lipase gene variants that may exist, and more particularly those found in SEQ ID NO:1.

### IV. Methods of Treatment

The present invention opens the door to a multitude of knew or improved treatment methods where it is desirable to detect and/or predict a subject intolerance to an hypolipidemic agent. Thus, some aspects, the present invention relates to such improved therapeutic methods, including methods which comprise the administration to a subject of an hypolipidemic agent such as a statin.

According to one particular aspect, the invention relates to a method for monitoring, treating, improving, or alleviating hyperlipidemia in a subject, the method comprising the step of detecting intolerance to an hypolipidemic agent in a subject receiving the hypolipidemic agent; and/or comprising the step of predicting an intolerance to an hypolipidemic agent prior to administration of the hypolipidemic agent to a subject. Such method may further comprise the step of reducing dosage of the hypolipidemic agent when intolerance thereto is detected and/or predicted.

For instance, statins form the class of medication which is most largely used in order to reduce the risk of cardiovascular atherosclerotic disease (MCA). They reduce the risk of MCA by 25 to 35% in different populations of patients (Schillinger M, Exner M, Mlekusch W, et al. Eur Heart J 2004; 25(9): 742-8). Generally, statins are well tolerated, but their use can sometimes be associated with a muscular toxicity more or less severe, varying between simple myalgia and rhabdomyolysis syndrome (severe muscular toxicity, presence of myoglobin in urine and renal insufficiency) (Thompson P D, Clarkson P, Karas R H. JAMA 2003; 289(13): 1681-90).

Even though rhabdomyolysis is a rare phenomenon which only manifests itself in under 1% of the patients treated with one or more statins, it is a severe condition which cause be a cause of death and has already been the lead to the withdrawal from the market of a statin, the cerivastatin (Baycol, Bayer) (Rosenson R S. Am J Med 2004; 116(6): 408-16). However, even in the absence of rhabdomyolysis, the presence of mild symptoms (myalgia) and moderate symptoms (moderate elevation of the plasmatic values of phosphokinase creatine (CK)) leads to important clinical consequences (increase in the number of medical appointments, a change in dosage or type or medications already used, etc., but also to important social consequences (reduced quality of life). The presence of side effects, even mild ones, therefore limits the use of statins, which are nevertheless very efficient.

The statins generally used in clinical practice for the treatment of hypercholesterolemia, are the rosuvastatin, the simvastatin, the atorvastatin, the pravastatin, the fluvastatin and the lovastatin. These different statins are used in clinical practice in doses varying between 5 to 80 mg, depending on the strength of the statins, on the lipidic profile and the MCA risk profile of the person to be treated.

It is common in clinical practice that different classes of lipid-lowering drugs are given in combination to patients. These combinations of lipid-lowering drugs are given when a patient does not attain the therapeutic target of LDL-cholesterol concentration recommended by the American and Canadian therapeutic guides (National Cholesterol Education Program (NCEP) Adult Treatment Panel III (ATPIII)). For example, patients who have a high risk of developing MCAs have to lower their LDL-cholesterol level under 2.5 mmol/L, which can be difficult to attain with statins alone. Another example concerns patients who have a very complex hypercholesterolemia, which do not react in a predictable way to statins. Statins are commonly combined, for example, with fibrates, with bile-acid sequestrants, with nicotinic acid or with the inhibitor of cholesterol absorption EZETIMIBE™ (Stone N., Eur Heart J Supplements 2002; 4 (Suppl J): J19-J22). Moreover, since people with high risk of MCA very often present different metabolic/physiological disturbances, they are treated with a combination of multiple drugs, for example lipid-lowering drugs with anti-hypertensives or oral anti-hyperglycaemic. Known side effects listed from patients who take statins include, among others: muscular effects, headaches, gastrointestinal problems, insomnia, fatigue and elevations in liver enzymes. These side effects generally appear within days of the start of a treatment using statins.

Known muscular side effects can be classified according the following common definitions (Rosenson R S. Am J Med 2004; 116(6): 408-16):
(1) myalgia, complaints, muscle stiffness or pain accompanied or not by low increases in levels of CK;
(2) muscle anomalies, increase in the levels of CK to more than 3 times the upper limit of normal values (22-173 U/L) with or without muscle pain;
(3) severe muscular dystrophy, muscle weakness and pain, accompanied by an increase in the levels of CK to more than 10 times the upper limit of normal values;
(4) rhabdomyolysis, muscle damage (destruction of striated muscle fibres) excretion of myoglobin in urine, CK values over 10,000 U/L. The myoglobinuria can lead to proteinuria and renal failure.

The term <<myopathy>> is used to talk in a general manner of muscle anomalies which are cause by statins, a disease or another cause. On the other hand, it may be useful to determine the basal values, i.e. the CL values before treatment and myoglobin and to consider these variables as continuous variables.

The factors associated with a higher risk of myopathy due to the use of statins are age, kidney failure, hypothyroidism, hereditary or acquired (injuries) muscle diseases, family history of myopathy (or hereditary after taking statins or fibrates), the combination with fibrates, exercise and alcohol abuse (Rosenson R S. Am J Med 2004; 116(6): 408-16). However, these risk factors cannot determine the risk in all patients taking statins. There are still not biomarkers to identify the risk of developing side effects associated with the use of one or more statins.

Accordingly another aspect of the invention relates to methods for monitoring, treating, improving, or alleviating hyperlipidemia in human subjects. For instance, it is known that side effects may occur whatever the dose of statin, depending of each individual's susceptibility to this type of drug. For example, rosuvastatin is given at a dosage varying between 5 and 40 mg, while atorvastatin is given at a dosage varying between 10 and 80 mg. The present invention may help in adjusting the dose of statin and finding the dosage that will reduce or eliminate side effects while maximizing a desired plasma lipid reduction.

According to another aspect, the invention relates to a method for selecting a desirable dose of an hypolipidemic agent administered to a subject, the method comprising monitoring glycerol plasma concentrations of the subject for detecting an intolerance to the hypolipidemic agent; and adjusting the dose administered to the subject for reducing or eliminating side effects while maximizing a desired plasma lipid reduction. Typically, intolerance is detected in the subject when the subject glycerol plasma concentrations is greater than about 0.2 mmol/L.

According to another aspect, the invention relates to improving currents methods for reducing risks of cardiovascular disease in a subject, these current methods comprising the administration of an hypolipidemic agent to the subject. The improvement according to the present invention comprises monitoring glycerol plasma concentrations of the subject for detecting an intolerance to the hypolipidemic agent. In the event of detection of an intolerance to said hypolipidemic agent the method further comprises reducing dosage of the hypolipidemic agent administered to the subject for reducing the subject glycerol plasma concentrations.

Another aspect of the present invention concerns method which uses the hepatic lipase gene variants as biomarkers in order to evaluate the efficiency, toxicity and/or inocuity of drugs (existing or in development) that are or may be useful in the treatment of cardiovascular atherosclerotic diseases (MCA). Such drugs include but are not limited to fibrates (e.g. Peroxisome Proliferator-Activated Receptor-alpha (PPARα) agonists which are associated with risks of myotoxicity and rhabnomyolosys), PPARγ agonists (associated with risks of edema), cholesterol esters transfer protein inhibitors (CETP) which are associated with risks of hypertension, and squalene synthase enzyme inhibitors.

### V. Probes, Primers and Kits

Additional aspects of the inventions concerns oligonucleotides such as probes and primers, solid support and kits comprising such oligonucleotides, for use in detecting and/or predicting intolerance of a subject to an hypolipidemic agent.

The oligonucleotides, probes, primers, solid supports and/or kits of the invention may be useful for the practice of the methods of the invention, particularly for diagnostic and/or therapeutic applications in humans according to the prediction and detection methods described hereinbefore.

In one embodiment the oligonucleotide is a nucleic acid probe which comprises at least 20, 25, 30 or 40 contiguous nucleotides of any one of SEQ ID NO: 4; SEQ ID NO: 5; SEQ ID NO:6; and SEQ ID NO: 7. Preferably the oligonucleotide comprises at least 5, 10, 15, or 20 contiguous nucleotides extending on each side of: the nucleotide at position 31 of SEQ ID NO: 4, SEQ ID NO: 5; SEQ ID NO: 6; or SEQ ID NO: 7. More preferably, the oligonucleotide comprises at least 30, 40, or 50 contiguous nucleotides of any one of SEQ ID NO: 4, SEQ ID NO: 5; SEQ ID NO: 6; or SEQ ID NO: 7. The invention also encompasses oligonucleotides which comprise at least 20 nucleotides sharing at least 80%, 85%, 90%, 95%, or 99% identity with SEQ ID NO: 4, SEQ ID NO: 5; SEQ ID NO: 6; or SEQ ID NO: 7 and hybridizing specifically thereto.

Those skilled in the art will know how to select oligonucleotides sequences that fill their particular needs. Probes will be specifically chosen to be used in different technique as in a PCR reaction (ex: U.S. Pat. Nos. 4,683,195 et 4,683,202), or alternatively using a <<ligation chain mutation>> (LCR) (P.N.A.S., 91: 360-364). This method may include the steps of obtaining a biological sample from a patient, isolate nucleic acids, put in contact the nucleic acids with one or several probes that hybridize specifically with the gene under specific conditions, detect the presence of absence of the amplification product, or detecting the size of the amplification product. It is anticipated that PCR and/or LCR may be desirable to use as a preliminary amplification step in conjunction with any of the techniques used for detecting mutations described herein.

In an alternative embodiment, mutations in a given gene can be identified by alterations in restriction enzyme cleavage patterns. For example, sample and DNA control are isolated amplified, digested with one or more restriction endonucleases, and fragment length sizes are determined by gel electrophoresis and compared. Differences in fragment length sizes between sample and control DNA indicate mutations in the sample DNA.

In other applications, mutations can be identified by hybridizing of a sample nucleic acids and control nucleic acids, ex., DNA et RNA, to high density arrays containing hundreds or thousands of oligonucleotide probes (Cronin, M. T. et al., (1996) Human Mutation, 7 :244-255; Kozal, M. J. et al., (1996) Nature Medicine, 2 : 753-759). For example, genetic mutations can be detected by a multiplex screening method using probes coupled to micro-beads following the steps of obtaining a DNA or RNA sample, amplify genome specific regions using the PCR technique, hybridize PCR products to the probes coupled to the micro-beads in favorable conditions, detect the signal using a LUMINEX™ instrument (technology Xmap).

In yet another embodiment, any of a variety of sequencing reactions known in the art can be used to directly sequence the gene and detect mutations by comparing the sequence of the gene from the sample with the corresponding wild-type (control) gene sequence. Example of sequencing reactions include those based automated techniques ((1995) Biotechniques, 19 :448), including sequencing using mass spectrometry (WO94/16101; Cohen et al. (1996) Adv. Chromatogr., 36: 127-162).

Table 1 herein after provides some examples of useful tagged (i.e. biotinylated) primers and probes according to the invention. The sequence of those primers and probes is as set forth in SEQ ID NOs: 8 to 15, respectively. Those primers and probes are specifically used for the genotyping of −514 C/T and using the Luminex™ technology.

TABLE 1
Examples of primers and probes

| | | | |
|---|---|---|---|
| TYPE | SITE | ORIENTATION | SEQUENCE |

| | | | |
|---|---|---|---|
| primer | −250 | 5′ | 5′/5Bio/ GCT GTC ACA GGG |
| | | | AGG CTT AG-3′ |
| primer | −250 | 3′ | 5′ TTC TTT ACT GCC CCC AAC |
| | | | TG-3′ |
| probe | −250 | anti-sense/ | 5′ /5UniAmM/ AAT TAA TCA |
| | | wild sequence | ACT TAA AGC TAC-3′ |
| probe | −250 | anti-sense/ | 5′ /5UniAmM AAT TAA TCA |
| | | variant sequence | ATT TAA AGC TAC-3′ |
| primer | −514 | 5′ | 5′-CCA TCT ATG GTC GCC TTT |
| | | | TC-3′ |
| primer | −514 | 3′ | 5′/5bio/ ATT GGT GAT GCT |
| | | | TGT GGT CA-3′ |
| probe | −514 | anti-sense | 5′-/5UniAmM/ CTT TTG ACA |
| | | wild sequence | CGG GGG TGA AG-3′ |
| probe | −514 | anti-sense | 5′-/5UniAmM CTT TTG ACA TGG |
| | | variant sequence | GGG TGA AG-3′ |

A solid support may comprise compound(s) for detecting and/or predicting intolerance of a subject to an hypolipidemic agent. In one embodiment, the compound is a nucleic acid probe designed for specific detection of hepatic lipase gene variant. The solid support may be a tube, a chip (see for instance Affimetrix GeneChip® technology), a membrane, a glass support, a filter, a tissue culture dish, a polymeric material, a bead, a silica support, etc. In preferred embodiments, the solid support comprises a plurality of probes each designed to specifically hybridize a different variant of the hepatic lipase gene.

A kit of the invention may comprise one or more of the following elements: a buffer for the homogenization of the biological sample(s), purified hepatic lipase proteins (and/or a fragment thereof) to be used as controls, incubation buffer(s), substrate and assay buffer(s), modulator buffer(s) and modulators (e.g. enhancers, inhibitors), standards, detection materials (e.g. antibodies, fluorescein-labelled derivatives, luminogenic substrates, detection solutions, scintillation counting fluid, etc.), laboratory supplies (e.g. desalting column, reaction tubes or microplates (e.g. 96- or 384-well plates), a user manual or instructions, etc. The kit and methods of the invention may be configured such as to permit a quantitative detection or measurement of the protein(s) or nucleotide of interest.

A kit for detecting and/or predicting intolerance of a subject to an hypolipidemic agent, the kit comprising a user manual or instructions and (i) a probe or primer specifically hybridizing to a nucleotide sequence comprising a hepatic lipase gene variant.

For instance, the kits may comprise at least one of: one or more oligonucleotides (e.g. a probe) which specifically hybridizes with a specific hepatic lipase gene variant, reaction buffers, and instructional material. Optionally, the oligonucleotide contains a detectable tag. Certain kits may contain a plurality of such oligonucleotides, which each serve to specifically hybridize a different variant of the hepatic lipase gene. Alternatively, the kit may comprise primers for amplifying specific regions of the hepatic lipase gene to allow for sequencing and identification of variants. The kits of the invention may also contain components of the amplification system, including PCR reaction materials such as buffers and a thermostable polymerase. In other embodiments, the kit of the present invention can be used in conjunction with commercially available amplification kits, such as may be obtained from GIBCO BRL (Gaithersburg, Md.) Stratagene (La Jolla, Calif.), Invitrogen (San Diego, Calif.). The kits may optionally include instructional material, positive or negative control reactions, templates, or markers, molecular weight size markers for gel electrophoresis, and the like.

Kits of the instant invention may also comprise antibodies immunologically specific for hepatic lipase protein(s) and/or variants thereof and instructional material. Optionally, the antibody contains a detectable tag. The kits may optionally include buffers for forming immunocomplexes, agents for detecting the immunocomplexes, instructional material, solid supports, positive or negative control samples, molecular weight size markers for gel electrophoresis, and the like.

### VI. Screening Methods

With the identification of a correlation between glycerol levels and side effects caused by hypolipidemic agents, and more particularly statins, it is now possible to use that knowledge in the screening, identification and development of new drugs. For example markers described herein could be used to evaluate the efficacy and the toxicity of drugs in development for the prevention of cardiovascular diseases.

The knowledge of the association between glycerol and HDL-triglycerides levels with statin intolerance could lead to the identification of new variants from genes that modulate their levels and therefore could be implicated in the mechanisms that induce statin muscle intolerance. Mutations identified in those genes could be integrated in multiplex arrays containing the HL variants already identified, in order to enhance the predictive value against statin intolerance.

### EXAMPLES

### Example 1

### Method of Detection of Statin Intolerance Using Glycerol as a Biomarker

As demonstrated in the present example, the inventors have established a connection between glycerolemia and muscle intolerance to statins among hypercholesterolemic subjects. At high levels, glycerol becomes a biomarker of intolerance to statins. The inventors have also determined a threshold value for glycerol levels: glycerol levels above that threshold value are considered abnormal and indicative of intolerance to statins.

Method

Subjects and Clinical Data

The study included 439 French-Canadian subjects from the Saguenay-Lac-Saint-Jean region (Quebec, Canada) who were treated with statins in order to reduce there cholesterol level.

The statins were administered alone or in combination with fibrates, bile-acid sequestrants, nicotinic acid or EZETIMIBE™, an inhibitor of cholesterol absorption.

During the treatment phase with one or more statins, all patients have received dietary advice by a nutritionist and were encouraged to follow a diet recommended by the <<American Heart Association>>. All patients were followed clinically and agreed to participate in studies on the genetic determinants of type 2 diabetes and on the cardiovascular diseases that combine genetic screening strategies and des études gènes candidats (Vohl M C, Lepage P, Gaudet D, Brewer C G, Betard C, Perron P, et al. J Lipid Research 2000; 41: 945-952; Gaudet D, Arsenault S, Perusse L, Vohl M C, St-Pierre J, Bergeron J, et al. Am J Hum Genet 2000; 66: 1558-1568).

The side effects reported by the patients in this study include muscle effects (muscle pain, elevations in the levels of CK, proteinuria, myoglobinuria), headaches, gastrointestinal problems, insomnia and fatigue.

Blood samples were collected after 12 hours of fasting. The particles of very-low-density lipoproteins (VLDL) (d<1.006 g/ml) were isolated by ultracentrifugation. The subfraction containing the HDL was obtained after precipitation of the LDL particles (d>1.006 g/ml) in the sub-natant with heparin and MnCl₂. The cholesterol, the triglycerides and CK levels were measured with the help of an enzymatic method with a Multiparity Analyzer CX7® (Beckman). Myoglobinuria was measured with the use of an immunologic dosage system ADVIA Centaur®, while proteinuria was determined with the help of a Synchron LX® analyzer (Beckman). Glycerol level was measured by calorimetric analysis (RANDOX Laboratories Ltd, Crumlin, UK) with the help of an Olympus AU400e™ camera (Diagnostic Systems Group, Melville, N.Y., USA).

Patients signed a consent form for this study and a code was assigned to each patient. That code allow to systematically anonymize all clinical data and make possible the construction of family trees and the analysis of family relations without having access to nominal information (Gaudet D, Arsenault S, Belanger C, Hudson T J, Perron P, Bernard M, et al. Clin Genet 1999; 55: 259-264.).

Statistical Analyses

Variance analysis were carried out in order to determine the effect of the different glycerol concentrations before treatment with one or more statins on the CK levels during the statins treatment.

The glycerolemia levels were determined according to the following categories: very high dysglycerolemia (glycerol >0.2 mmol/l), high dysglycerolemia (glycerol 0.1-0.2 mmol/l), mildly high dysglycerolemia (glycerol 0.08-0.1 mmol/l), normal-lightly high glycerolemia (0.05-0.08 mmol/l) and normal glycerolemia (glycerol <0.05 mmol/l). Bonferroni's test was used to determine the degree of significance of the differences between the groups. All analyses were carried out with the use of test kit SPSS™ (version 11.0, SPSS, Chicago, Ill.).

Results

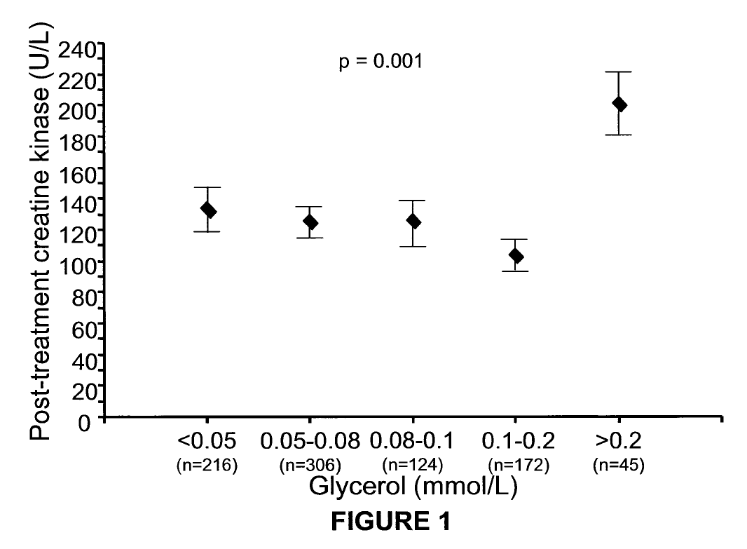

FIG. 1 shows CK levels during treatment with statins according to different glycerol concentrations before the treatment with one or more statins. The glycerol levels are categorized in five (5) groups: (1) <0.05, (2) 0.05-0.08, (3) 0.08-0.1, (4) 0.1-0.2, and (5) >0.2 mmol/L.

A significant relationship was observed between pre-treatment plasma glycerol concentrations and post-treatment CK levels (p<0.001). Moreover, the CK values were significantly lower among subjects having a glycerolemia between 0.1 and 0.2 mmol/L compared to subjects having a glycerolemia <0.05 or between 0.05 and 0.08 mmol/L. By contrast, the subjects who had a glycerolemia >0.2 had higher CK values compared to the other groups.

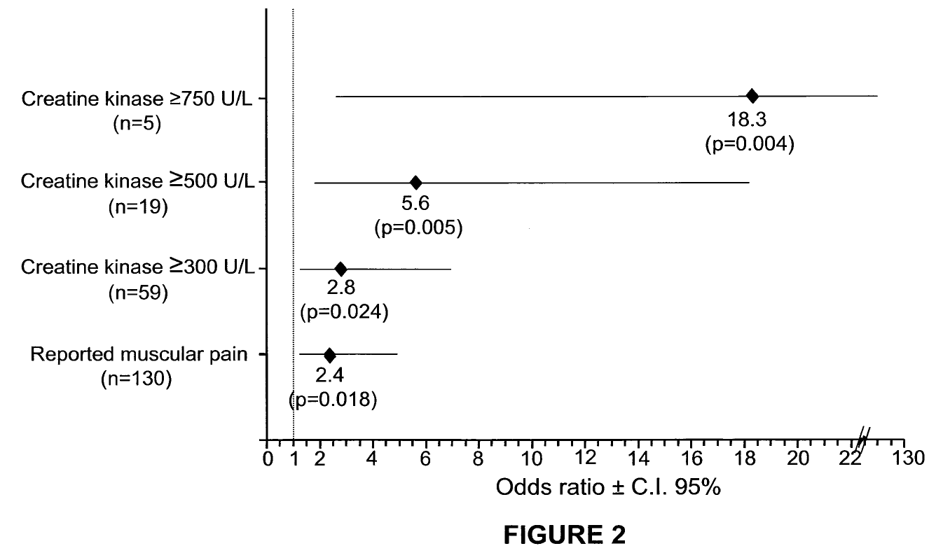

As shown in FIG. 2, pre-treatment plasma glycerol concentration >0.2 mmol/L was associated with (1) increased risk of post-treatment muscular complaint (p=0.018) (2) increased risk of post-treatment CK levels ≧3 times upper limit of normal (p=0.024), (3) increased risk of post-treatment CK levels ≧5 times upper limit of normal (p=0.005) and increased risk of CK levels ≧7.5 times upper limit of normal (p=0.004).

It may be noted that glycerol seems to protect, up to a certain threshold, against the CK elevations which occur during the treatment with the statins. From a glycerolemia of about >0.2 mmol/L, glycerol no longer protects against CK elevations and becomes a biomarker of intolerance to statins as demonstrated by the higher CK values.

### Example 2

### Statin Intolerance Detection Method Using HDL-Triglycerides Levels and Hepatic Lipase Gene Variant as Biomarkers

This study was carried out to determine the relationship between the composition HDL particles and the expression of side effects in patients receiving a treatment with statins. Multivaried analyses were made to evaluate the association between the cholesterol and triglyceride content of the HDLs and known markers of statin intolerance (muscular and non-muscular side effects, plasma concentration of creatine kinase (CPK), myoglobinuria). Some frequent candidate gene variants were also studied.

Method

Subjects and Clinical Data

This study included 670 French Canadian subjects (425 men and 245 women), natives of the Saguenay-Lac-Saint-Jean region (Quebec, Canada), who had been treated with different dosages of statins in order to reduce their cholesterol level. Statins were administered alone or in combination with fibrates, bile-acid sequestering agents, nicotinic acid or a cholesterol absorption inhibitor called EZETIMIBE™. During the statin treatment phase, all subjects received dietary counselling from a nutritionist and were advised to follow a diet recommended by the American Heart Association. All the patients were clinically followed at the Clinique des Lipides of the Centre de Santé et des Services Sociaux de Chicoutimi (CSSS Chicoutimi). The subjects agreed to participate in studies on the genetic determinants of type 2 diabetes and cardiovascular disease that combine genetic screening strategies and candidate gene studies according to what was described in Vohl M C, Lepage P, Gaudet D, Brewer C G, Betard C, Perron P, et al. “Molecular scanning of the human Peroxisome Proliferator-Activated Receptor-gamma gene: association of the L162V mutation with hyperapobetalipoproteinemia”, (2000) 40 J. Lipid Research, 945-952 and Gaudet D, Arsenault S, Perusse L, Vohl M C, St-Pierre J, Bergeron J, et al. “Glycerol as a correlate of impaired glucose tolerance: dissection of a complex system by use of a simple genetic trait”, (2000) 66 Am. J. Hum. Genet., 1558-1568.

The side effects reported by the subjects in this study included muscle effects (muscle pain, elevations of creatine kinase (CK) levels, proteinuria and myoglobinuria), headaches, gastrointestinal problems, insomnia and fatigue.

Blood samples were taken after 12 hours of fasting. The very low density lipoproteins (VLDL) (d<1.006 g/ml) were isolated by ultracentrifugation. The sub-fraction containing the HDLs was obtained after precipitation of the LDL particles (d>1.006 g/ml) in a sub-natant with heparin and MnCl₂ using the method described in Havel R J, Eder H A, Bragdon J H. “The distribution and chemical composition of ultracentrifugally separated lipoproteins in human serum,” (1955). 34 J. Clin. Invest., 1345-1354.

The cholesterol, triglycerides and CK levels were measured according to an enzymatic method using a Multiparity Analyzer CX7™ (Beckman). Myoglobinuria was measured using an ADVIA CENTAUR™ assay system, while proteinuria was determined using a Synchron LX™ analyzer (Beckman). The presence of −514 C/T polymorphism was identified by fluorescence polarization detection according to the method described in Kwok P Y. “SNP genotyping with fluorescence polarization detection”, (20002) 19 Hum. Mut., 315-323.

The subjects signed a consent form for this study and a code was assigned to each subject, which systematically denominalized all clinical data and allowed the construction of family trees and the analysis of family relationships without access to the nominal information, according to the method described in Gaudet D., Arsenault S., Belanger C., Hudson T. J., Perron P., Bernard M., et al. “Procedure to protect confidentiality of familial data in community genetics and genomic research”, (1999) 55 Clin. Genet. 259-264. This project was approved by the CSSS de Chicoutimi Research Ethics Board.

Statistical Analyses

Correlations were made between the levels of HDL triglycerides before statin treatment and CPK levels during statin treatment as related to the presence of various genic variants in the hepatic lipase gene. More specifically, the analysis was directed to the effect of the presence of the 514 CC allele compared to the presence of the 514 CT and 514 TT alleles to the observed relationship of the levels of HDL-TG before treatment and the levels of CK during treatment. The plasma levels of HDL-TG underwent a logarithmic transformation before the statistical analyses. The analyses were controlled for age, gender and waist size. All statistical analyses were conducted using the SPSS™ analysis kit (version 11.0, SPSS, Chicago Ill.).

Results

All together, 21.6% of the subjects treated with statins reported a side effect, all side effects combined. Muscle pains were reported by 19.7% of subjects but only 2% of had muscle pains accompanied by elevations of CK levels exceeding more than 3 times the normal upper limit (Table 2). The side effects appeared quickly, generally within two weeks of the start of treatment.

TABLE 2
Subjects' characteristics

| | |
|---|---|
| Men/Women | 425/245 |
| Age (years ± S.D.) | 49.9 ± 10.0 |
| Daily dose of statins: | |
| Quartile 1^{§} | 20.1 |
| Quartile 2^{§} | 30.6 |
| Quartile 3^{§} | 22.1 |
| Quartile 4^{§} | 27.2 |
| Use in combination (%)* | 12.4 |
| Reported side effects (all) (%) | 21.6 |
| Reported muscle pain (%)¹ | 19.7 |
| Créatine kinase >300 U/L (%)² | 7.0 |
| Créatine kinase >500 U/L (%)² | 2.0 |

*Combinations of statins with fibrates, bile-acid sequestrant, nicotinic acid or cholesterol absorption inhibitor EZETIMIBE ™.
¹n = 654;
²n = 639;
³n = 460.
^{§}Quartile 1 = Crestor, Zocor, Lipitor 5 mg and Mevacor, Prevachol, Zocor, Lipitor 10 mg;
^{§}Quartile 2 = Lescol, Mevacor, Pravachol, Zocor, Lipitor 20 mg; Baycol 2 mg; Crestor 10 mg
^{§}Quartile 3 = Lescol, Lipitor, Mevacor, Zocor 40; Lipitor 60
^{§}Quartile 4 = Mevacor, Pravachol, Zocor, Lipitor 80 mg; Crestor 40 mg et statins in combinations with hypolipidemic agents.

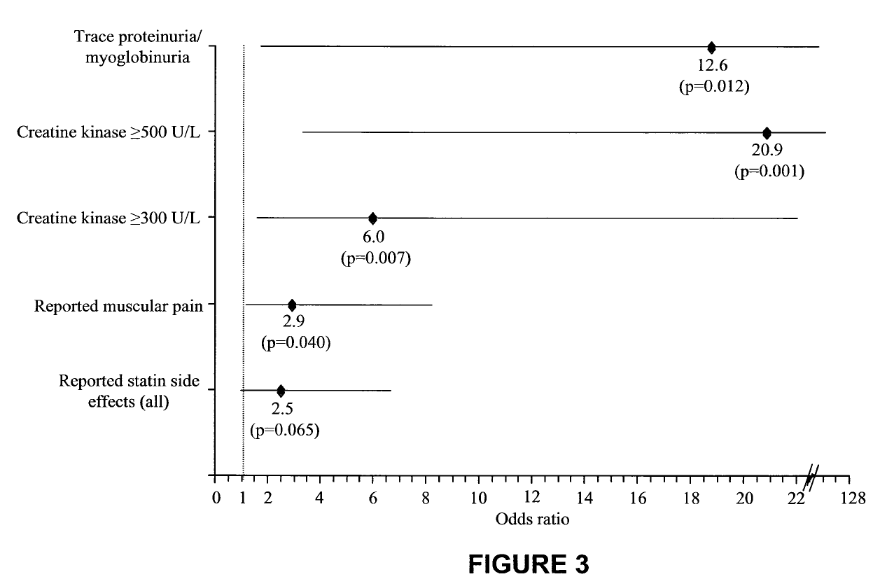

The HDL-TG plasma level was associated with a high risk of showing muscular and non-muscular side effects (the ratio of scores (OR=2.9; p=0.04), an increase of CPK levels ≧300 U/L (OR=6.0, p=0.007), and an increase in CPK levels ≧500 U/L (OR=20.9, p=0.001) or proteinuria (OR=12.6, p=0.012)—See FIG. 3).

The basal TG concentration in the LDL particles defined a severity gradient for the statin-induced myotoxicity. Thus, higher the HDL-TG levels were before statins treatment, higher were the risks for the patients of having a severe myotoxicity during the treatment. There results suggest that a determination of the TG content of the HDL particles is an important prevention target among individuals treated with medicine comprising statins.

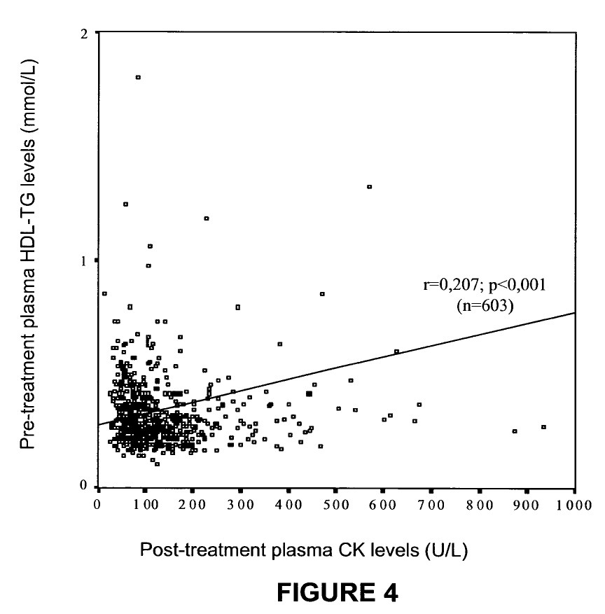

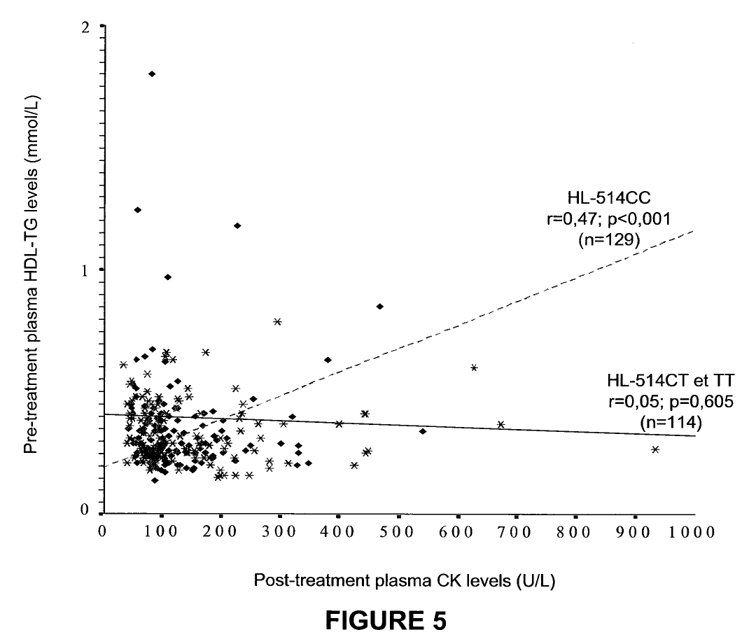

A positive relation was observed between the HDL-triglycerides plasma concentration before treatment and the CK concentration during treatment (FIG. 4). However, the relation between the HDL-TG levels and the CK levels was no more detected in subjects carrying the 514 CT and 514 TT alleles. On the other hand, that relation was very strong in subjects carrying the 514 CC (FIG. 5).

Even is preferred embodiments of the invention have been described in details hereinabove and illustrated in the accompanying drawings, it is to be understood that the invention is not limited to these particular embodiments and that several changes and modifications could be made without departing from the scope or spirit of the present invention.

1. A method for detecting an intolerance in a subject receiving an hypolipidemic agent, comprising assessing a plasma sample from said subject for measuring therein the fastening concentration of a lipidic biomarker and correlating said concentration with a threshold value, wherein an abnormal concentration of said lipidic biomarker is indicative of an intolerance to the hypolipidemic agent.

2. The method of claim 1, wherein the lipidic biomarker is glycerol, and wherein an abnormal concentration of glycerol is a concentration greater than the threshold value.

3. The method of claim 1, wherein an abnormal concentration of glycerol is a fastening concentration greater than about 0.2 mmol/L.

4. The method of claim 1, wherein the lipidic biomarker is HDL-triglycerides, and wherein an abnormal concentration of HDL-triglycerides is a concentration greater than the threshold concentration.

5. The method of claim 4, wherein an abnormal concentration of HDL-triglycerides is a concentration greater than about 0.15 mmol/L.

6. The method of claim 1, wherein the hypolipidemic agent is a statin.

7. The method of claim 6, wherein the statin is selected from the group consisting of rosuvastatin, simvastatin, atorvastatin, pravastatin, fluvastatin and lovastatin.

8. A method for detecting muscular intolerance to an hypolipidemic agent in a subject receiving said hypolipidemic agent, comprising assessing said subject resistance to glycerol.

9. The method of claim 8, wherein the hypolipidemic agent is a statin.

10. The method of claim 9, wherein the statin is selected from the group consisting of rosuvastatin, simvastatin, atorvastatin, pravastatin, fluvastatin and lovastatin.

11. The method of claim 8, wherein subject resistance to glycerol is defined by a fastening plasma glycerol concentration greater than about 0.2 mmol/L.

12. A method for monitoring, treating, improving, or alleviating hyperlipidemia in a subject, the method comprising the step of detecting intolerance to an hypolipidemic agent in a subject receiving said hypolipidemic agent; and/or the step of predicting an intolerance to an hypolipidemic agent prior to administration of said hypolipidemic agent to a subject.

13. The method of claim 13, further comprising the step of reducing dosage of the hypolipidemic agent when intolerance to said hypolipidemic agent is detected and/or predicted.

14. A method for selecting a desirable dose of an hypolipidemic agent administered to a subject, the method comprising monitoring fastening glycerol plasma concentrations of said subject for detecting an intolerance to said hypolipidemic agent; and adjusting said dose for reducing or eliminating side effects while maximizing a desired plasma lipid reduction.

15. The method of claim 14, wherein intolerance is detected when said subject fastening glycerol plasma concentrations is greater than about 0.2 mmol/L.

16. A method of detecting intolerance of a subject to an hypolipidemic agent, comprising assessing a biological sample from said subject for identifying a hepatic lipase gene variant, wherein presence of said hepatic lipase gene variant is predictive of a reduced risk intolerance to hypolipidemic agents.

17. The method of claim 16, wherein said variant is present in a promoter region of the hepatic lipase gene.

18. The method of claim 16, wherein said variant is selected from the group consisting of: an adenine at position 2764 of SEQ ID NO:3; (ii) a thymine at position 2500 of SEQ ID NO:3; (iii) a cytosine at position 2304 of SEQ ID NO:3; and (iv) a guanine at position 2764 of SEQ ID NO:3.

19. The method of claim 16, wherein the hypolipidemic agent is a statin.

20. A nucleic acid probe for predicting intolerance of a subject to an hypolipidemic agent, said probe comprising at least 20 contiguous nucleotides of any one of SEQ ID NO: 4; SEQ ID NO: 5; SEQ ID NO:6; and SEQ ID NO: 7, and wherein said probe comprises at least 10 contiguous nucleotides extending on each side of nucleotide at position 31 of SEQ ID NO: 4, SEQ ID NO: 5; SEQ ID NO: 6; or SEQ ID NO: 7.

21. A solid support comprising a probe according to claim 20.

22. A kit for detecting and/or predicting intolerance of a subject to an hypolipidemic agent, the kit comprising a user manual or instructions and (i) a probe or primer specifically hybridizing to a nucleotide sequence comprising a hepatic lipase gene variant.
